(19) United States
(12) Patent Application Publication
(10) Pub. No.: US 2022/0223299 A1
(43) Pub. Date: Jul. 14, 2022

(54) METHODS AND APPARATUSES FOR MODELING, SIMULATING, AND TREATING HEREDITARY ANGIOEDEMA

(71) Applicant: Takeda Pharmaceutical Company Limited, Osaka (JP)

(72) Inventors: Rangaraj Narayanan, Cambridge, MA (US); Hoa Q. Nguyen, Lynnfield, MA (US); Daniel J. Sexton, Melrose, MA (US)

(73) Assignee: Takeda Pharmaceutical Company Limited, Osaka (JP)

(21) Appl. No.: 17/613,465

(22) PCT Filed: May 22, 2020
(86) PCT No.: PCT/US2020/034196

§ 371 Date: Nov. 22, 2021

Related U.S. Application Data
(60) Provisional application No. 62/988,285, filed on Mar. 11, 2020.
(60) Provisional application No. 62/852,189, filed on May 23, 2019.

(51) Int. Cl. G16H 70/60; G16H 50/50; G16H 50/30

(52) U.S. Cl. CPC G16H 70/60; G16H 50/30; G16H 50/50

(57) ABSTRACT

Aspects of the present application provide for methods and apparatuses for modeling, simulating, and treating hereditary angioedema (HAE). According to some aspects, a quantitative systems pharmacology (QSP) model is provided for simulating the efficacy of drug intervention under context of HAE pathophysiology. The QSP model may comprise a plurality of individual models including one or more PK models and/or one or more PD models for simulating drug exposure, target engagements and acute attack rate in HAE patients. A virtual patient population representing a plurality of virtual patients may be developed and input into the QSP model for executing a virtual clinical trial. In some embodiments, the QSP model may be used evaluate a response of the contact system and/or an effectiveness of a therapeutic intervention for treating HAE.

### CROSS-REFERENCE TO RELATED APPLICATIONS

This application claims the benefit under 35 U.S.C. under § 119(e) of U.S. Provisional Application Ser. No. 62/852,189 titled “METHODS AND APPARATUSES FOR MODELING, SIMULATING, AND TREATING HEREDITARY ANGIOEDEMA” and filed on May 23, 2019 under Attorney Docket No. D0617.70130US00 and U.S. Provisional Application Ser. No. 62/988,285 titled “METHODS AND APPARATUS FOR MODELING, SIMULATING, AND TREATING HEREDITARY ANGIOEDEMA USING PKA INHIBITORS” and filed on Mar. 11, 2020 under Attorney Docket No. D0617.70135US00, each of which is incorporated by reference in its entirety herein.

### BACKGROUND

Hereditary angioedema (HAE) is an autosomal dominant disease caused by problems in the C1 inhibitor protein. HAE type I is characterized by a deficiency in the C1 inhibitor protein while HAE type II is characterized by dysfunction in the C1 inhibitor protein. HAE affects an estimated 1 in 67,000 people worldwide. HAE manifests clinically as unpredictable, intermittent attacks of subcutaneous or submucosal oedema (swelling) of the face, larynx, gastrointestinal tract, limbs and/or genitalia. The underlying mechanism is due to the excess activation of the ‘contact system’ where plasma kallikrein acts on high molecular weight kininogen (HMWK), leading to bradykinin release, causing vasodilation due to binding of bradykinin to B2 receptors on endothelial cells.

### BRIEF SUMMARY

Some embodiments provide for a computer-implemented method for modeling and simulating hereditary angioedema (HAE), comprising: obtaining a quantitative systems pharmacology (QSP) model of HAE, wherein the QSP model is configured to represent autoactivation of Factor XII by elevating levels of FXIIa in response to an indication that a trigger has been input into the QSP model; determining disease predictive descriptors; assigning the disease predictive descriptors to a virtual patient population; and processing the virtual patient population using the QSP model to provide processed data, wherein the processed data comprises an amount of one or more contact system proteins.

Some embodiments provide for a system comprising: at least one computer hardware processor; and at least one non-transitory computer-readable storage medium storing processor executable instructions that, when executed by the at least one computer hardware processor, cause the at least one computer hardware processor to perform a computer-implemented method for modeling and simulating hereditary angioedema (HAE), comprising: obtaining a quantitative systems pharmacology (QSP) model of HAE, wherein the QSP model is configured to represent autoactivation of Factor XII by elevating levels of FXIIa in response to an indication that a trigger has been input into the QSP model; determining disease predictive descriptors; assigning the disease predictive descriptors to a virtual patient population; and processing the virtual patient population using the QSP model to provide processed data, wherein the processed data comprises an amount of one or more contact system proteins.

Some embodiments provide for at least one non-transitory computer-readable medium storing processor executable instructions that, when executed by at least one computer hardware processor, cause the at least one computer hardware processor to perform a computer implemented method for modeling and simulating hereditary angioedema (HAE), the comprising: obtaining a quantitative systems pharmacology (QSP) model of HAE, wherein the QSP model is configured to represent autoactivation of Factor XII by elevating levels of FXIIa in response to an indication that a trigger has been input into the QSP model; determining disease predictive descriptors; assigning the disease predictive descriptors to a virtual patient population; and processing the virtual patient population using the QSP model to provide processed data, wherein the processed data comprises an amount of one or more contact system proteins.

Some embodiments provide for a computer-implemented method for determining a trigger strength by estimating one or more characteristics of a contact system in a patient in response to a trigger, the method comprising: obtaining a quantitative systems pharmacology (QSP) model of hereditary angioedema (HAE), wherein the QSP model is configured to represent autoactivation of Factor XII by elevating levels of FXIIa in response to an indication that the trigger has been input into the QSP model; calibrating the QSP model with known data; inputting the trigger into the QSP model, the trigger being configured to generate FXIIa by causing Factor XII of the contact system to autoactivate; obtaining, from the QSP model, an amount of a protein of the contact system generated in response to the trigger.

Some embodiments provide for a system, comprising: at least one computer hardware processor; and at least one non-transitory computer-readable storage medium storing processor executable instructions that, when executed by the at least one computer hardware processor, cause the at least one computer hardware processor to perform a computer-implemented method for estimating one or more characteristics of a contact system in a patient in response to a trigger, the method comprising: obtaining a quantitative systems pharmacology (QSP) model of hereditary angioedema (HAE), wherein the QSP model is configured to represent autoactivation of Factor XII by elevating levels of FXIIa in response to an indication that the trigger has been input into the QSP model; calibrating the QSP model with known data; inputting the trigger into the QSP model, the trigger being configured to generate FXIIa by causing Factor XII of the contact system to autoactivate; obtaining, from the QSP model, an amount of a protein of the contact system generated in response to the trigger.

Some embodiments provide for at least one non-transitory computer-readable storage medium storing processor executable instructions that, when executed by at least one computer hardware processor, cause the at least one computer hardware processor to perform a computer-implemented method for estimating one or more characteristics of a contact system in a patient in response to a trigger, the method comprising: obtaining a quantitative systems pharmacology (QSP) model of hereditary angioedema (HAE), wherein the QSP model is configured to represent autoactivation of Factor XII by elevating levels of FXIIa in response to an indication that the trigger has been input into the QSP model; calibrating the QSP model with known data; inputting the trigger into the QSP model, the trigger being configured to generate FXIIa by causing Factor XII of the contact system to autoactivate; obtaining, from the QSP model, an amount of a protein of the contact system generated in response to the trigger.

Some embodiments provide for a computer-implemented method for determining a relationship between hereditary angioedema (HAE) attack frequency and Factor XII trigger rate, the method comprising: obtaining a quantitative systems pharmacology (QSP) model of HAE, wherein the QSP model is configured to represent autoactivation of Factor XII by elevating levels of FXIIa in response to an indication that a trigger has been input into the QSP model; assigning a Factor XII trigger rate for one or more patients in a virtual patient population, wherein the Factor XII trigger rate comprises a rate at which autoactivation of Factor XII is triggered in the QSP model; applying the QSP model to the one or more patients in the virtual patient population to obtain processed data, wherein the processed data comprises an amount of one or more contact system proteins; determining an HAE attack frequency for the one or more patients in the virtual patient population based on the processed data; and determining a relationship between HAE attack frequency and Factor XII trigger rate.

Some embodiments provide for a system, comprising: at least one computer hardware processor; and at least one non-transitory computer-readable storage medium storing processor executable instructions that, when executed by at least one computer hardware processor, cause the at least one computer hardware processor to perform a computer-implemented method for determining a relationship between hereditary angioedema (HAE) attack frequency and Factor XII trigger rate, the method comprising obtaining a quantitative systems pharmacology (QSP) model of HAE, wherein the QSP model is configured to represent autoactivation of Factor XII by elevating levels of FXIIa in response to an indication that a trigger has been input into the QSP model; assigning a Factor XII trigger rate for one or more patients in a virtual patient population, wherein the Factor XII trigger rate comprises a rate at which autoactivation of Factor XII is triggered in the QSP model; applying the QSP model to the one or more patients in the virtual patient population to obtain processed data, wherein the processed data comprises an amount of one or more contact system proteins; determining an HAE attack frequency for the one or more patients in the virtual patient population based on the processed data; and determining a relationship between HAE attack frequency and Factor XII trigger rate.

Some embodiments provide for at least one non-transitory computer-readable storage medium storing processor executable instructions that, when executed by at least one computer hardware processor, cause the at least one computer hardware processor to perform a computer-implemented method for determining a relationship between hereditary angioedema (HAE) attack frequency and Factor XII trigger rate, the method comprising: obtaining a quantitative systems pharmacology (QSP) model of HAE, wherein the QSP model is configured to represent autoactivation of Factor XII by elevating levels of FXIIa in response to an indication that a trigger has been input into the QSP model; assigning a Factor XII trigger rate for one or more patients in a virtual patient population, wherein the Factor XII trigger rate comprises a rate at which autoactivation of Factor XII is triggered in the QSP model; applying the QSP model to the one or more patients in the virtual patient population to obtain processed data, wherein the processed data comprises an amount of one or more contact system proteins; determining an HAE attack frequency for the one or more patients in the virtual patient population based on the processed data; and determining a relationship between HAE attack frequency and Factor XII trigger rate.

Some embodiments provide for a computer-implemented method for determining an effectiveness of an administered drug in treating hereditary angioedema (HAE), the method comprising: determining pharmacokinetic parameters of the administered drug for a virtual patient population; determining disease predictive descriptors for the virtual patient population; assigning the pharmacokinetic parameters and disease predictive descriptors to the virtual patient population; processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and using the processed data to obtain an indicator of the effectiveness of the administered drug on treating HAE.

Some embodiments provide for a system, comprising: at least one computer hardware processor; and at least one non-transitory computer-readable storage medium storing processor-executable instructions that, when executed by at least one computer hardware processor, cause the at least one computer-hardware processor to perform a method for determining an effectiveness of an administered drug in treating hereditary angioedema (HAE), the method comprising: determining pharmacokinetic parameters of the administered drug for a virtual patient population; determining disease predictive descriptors for the virtual patient population; assigning the pharmacokinetic parameters and disease predictive descriptors to the virtual patient population; processing the virtual patient population using a quantitative systems pharmacology (QSP) of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and using the processed data to obtain an indicator of the effectiveness of the administered drug on treating HAE.

Some embodiments provide for at least one non-transitory computer-readable storage medium storing processor-executable instructions that, when executed by at least one computer hardware processor, cause the at least one computer-hardware processor to perform a method for determining an effectiveness of an administered drug in treating hereditary angioedema (HAE), the method comprising: determining pharmacokinetic parameters of the administered drug for a virtual patient population; determining disease predictive descriptors for the virtual patient population; assigning the pharmacokinetic parameters and disease predictive descriptors to the virtual patient population; processing the virtual patient population using a quantitative systems pharmacology (QSP) model of HAE, wherein the QSP model is configured to represent autoactivation of Factor XII by elevating levels of FXIIa in response to an indication that a trigger has been input into the QSP model and the processed data comprises an amount of one or more contact system proteins; and using the processed data to obtain an indicator of the effectiveness of the administered drug on treating HAE.

Some embodiments provide for a computer-implemented method for determining an effectiveness of a dosage of an administered drug in treating hereditary angioedema (HAE), the method comprising: determining pharmacokinetic parameters of the dosage of the administered drug for a virtual patient population; determining disease predictive descriptors for the virtual patient population; assigning the pharmacokinetic parameters and disease predictive descriptors to the virtual patient population; processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and using the processed data to obtain an indicator of the effectiveness of the dosage of the administered drug on treating HAE.

Some embodiments provide for a system, comprising: at least one computer-hardware processor; and at least one non-transitory computer-readable storage medium storing processor-executable instructions that, when executed by the at least one computer-hardware processor, cause the at least one computer-hardware processor to perform a method for determining an effectiveness of a dosage of an administered drug in treating hereditary angioedema (HAE), the method comprising: determining pharmacokinetic parameters of the dosage of the administered drug for a virtual patient population; determining disease predictive descriptors for the virtual patient population; assigning the pharmacokinetic parameters and disease predictive descriptors to the virtual patient population; processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and using the processed data to obtain an indicator of the effectiveness of the dosage of the administered drug on treating HAE.

Some embodiments provide for at least one non-transitory computer-readable storage medium storing processor-executable instructions that, when executed by at least one computer-hardware processor, cause the at least one computer-hardware processor to perform a method for determining an effectiveness of a dosage of an administered drug in treating hereditary angioedema (HAE), the method comprising: determining pharmacokinetic parameters of the dosage of the administered drug for a virtual patient population; determining disease predictive descriptors for the virtual patient population; assigning the pharmacokinetic parameters and disease predictive descriptors to the virtual patient population; processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and using the processed data to obtain an indicator of the effectiveness of the dosage of the administered drug on treating HAE.

Some embodiments provide for a computer-implemented method for determining an effect of non-adherence to a dosing regimen of an administered drug in treating hereditary angioedema (HAE), the method comprising: determining pharmacokinetic parameters of the administered drug for a virtual patient population, wherein the pharmacokinetic parameters include a frequency of non-adherence to the dosing regimen; determining disease predictive descriptors for the virtual patient population; assigning the pharmacokinetic parameters and disease predictive descriptors to the virtual patient population; processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and using the processed data to determine an effect of the frequency of non-adherence on treating HAE.

Some embodiments provide for a system, comprising: at least one computer hardware processor; and at least one non-transitory computer-readable medium storing processor-executable instructions that, when executed by the at least one computer-hardware processor, cause the at least one computer-hardware processor to perform a method for determining an effect of non-adherence to a dosing regimen of an administered drug in treating hereditary angioedema (HAE), the method comprising: determining pharmacokinetic parameters of the administered drug for a virtual patient population, wherein the pharmacokinetic parameters include a frequency of non-adherence to the dosing regimen; determining disease predictive descriptors for the virtual patient population; assigning the pharmacokinetic parameters and disease predictive descriptors to the virtual patient population; processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and using the processed data to determine an effect of the frequency of non-adherence on treating HAE.

Some embodiments provide for at least one non-transitory computer-readable storage medium storing processor-executable instructions that, when executed by at least one computer-hardware processor, cause the at least one computer-hardware processor to perform a method for determining an effect of non-adherence to a dosing regimen of an administered drug in treating hereditary angioedema (HAE), the method comprising: determining pharmacokinetic parameters of the administered drug for a virtual patient population, wherein the pharmacokinetic parameters include a frequency of non-adherence to the dosing regimen; determining disease predictive descriptors for the virtual patient population; assigning the pharmacokinetic parameters and disease predictive descriptors to the virtual patient population; processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and using the processed data to determine an effect of the frequency of non-adherence on treating HAE.

Some embodiments provide for a computer-implemented method for determining an amount of a protein of a contact system in a patient in response to administration of a drug for treating hereditary angioedema (HAE), the method comprising: determining pharmacokinetic parameters of the drug for a virtual patient population; determining disease predictive descriptors for the virtual patient population; assigning the pharmacokinetic parameters and disease predictive descriptors to the virtual patient population; processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model; and determining the amount of the protein based on the processed data.

Some embodiments provide for a system, comprising: at least one computer-hardware processor; and at least one non-transitory computer-readable storage medium storing processor-executable instructions that, when executed by the at least one computer-hardware processor, cause the at least one computer-hardware processor to perform a method for determining an amount of a protein of a contact system in a patient in response to administration of a drug for treating hereditary angioedema (HAE), the method comprising: determining pharmacokinetic parameters of the drug for a virtual patient population; determining disease predictive descriptors for the virtual patient population; assigning the pharmacokinetic parameters and disease predictive descriptors to the virtual patient population; processing the virtual patient population using a quantitative systems pharmacology (QSP) model of HAE obtain processed data, wherein the QSP model is configured to represent autoactivation of Factor XII by elevating levels of FXIIa in response to an indication that a trigger has been input into the QSP model; and determining the amount of the protein based on the processed data.

Some embodiments provide for at least one non-transitory computer-readable storage medium storing processor-executable instructions that, when executed by at least one computer-hardware processor, cause the at least one computer-hardware processor to perform a method for determining an amount of a protein of a contact system in a patient in response to administration of a drug for treating hereditary angioedema (HAE), the method comprising: determining pharmacokinetic parameters of the drug for a virtual patient population; determining disease predictive descriptors for the virtual patient population; assigning the pharmacokinetic parameters and disease predictive descriptors to the virtual patient population; processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model; and determining the amount of the protein based on the processed data.

Some embodiments provide for a computer-implemented method for determining a temporal profile illustrating an effect of a drug on a contact system in a patient, the method comprising: determining pharmacokinetic parameters of the drug for a virtual patient population; determining disease predictive descriptors for the virtual patient population; assigning the pharmacokinetic parameters and disease predictive descriptors to the virtual patient population; processing the virtual patient population using a quantitative systems pharmacology (QSP) model of hereditary angioedema (HAE) to obtain processed data, wherein the QSP model is configured to represent autoactivation of Factor XII by elevating levels of FXIIa in response to an indication that a trigger has been input into the QSP model and the processed data comprises an amount of one or more contact system proteins; and using the processed data to determine a measure of an amount of one or more proteins of the contact system over time in response to the drug.

Some embodiments provide for a system, comprising: at least one computer-hardware processor; and at least one non-transitory computer-readable storage medium storing processor-executable instruction that, when executed by the at least one computer-hardware processor, cause the at least one computer-hardware processor to perform a method for determining a temporal profile illustrating an effect of a drug on a contact system in a patient, the method comprising: determining pharmacokinetic parameters of the drug for a virtual patient population; determining disease predictive descriptors for the virtual patient population; assigning the pharmacokinetic parameters and disease predictive descriptors to the virtual patient population; processing the virtual patient population using a quantitative systems pharmacology (QSP) model of hereditary angioedema (HAE) to obtain processed data, wherein the QSP model is configured to represent autoactivation of Factor XII by elevating levels of FXIIa in response to an indication that a trigger has been input into the QSP model and the processed data comprises an amount of one or more contact system proteins; and using the processed data to determine a measure of an amount of one or more proteins of the contact system over time in response to the drug.

Some embodiments provide for at least one non-transitory computer-readable storage medium storing processor-executable instruction that, when executed by at least one computer-hardware processor, cause the at least one computer-hardware processor to perform a method for determining a temporal profile illustrating an effect of a drug on a contact system in a patient, the method comprising: determining pharmacokinetic parameters of the drug for a virtual patient population; determining disease predictive descriptors for the virtual patient population; assigning the pharmacokinetic parameters and disease predictive descriptors to the virtual patient population; processing the virtual patient population using a quantitative systems pharmacology (QSP) model of hereditary angioedema (HAE) to obtain processed data, wherein the QSP model is configured to represent autoactivation of Factor XII by elevating levels of FXIIa in response to an indication that a trigger has been input into the QSP model and the processed data comprises an amount of one or more contact system proteins; and using the processed data to determine a measure of an amount of one or more proteins of the contact system over time in response to the drug.

Some embodiments provide for a computer-implemented method for determining a characteristic of a hereditary angioedema (HAE) flare-up in response to administering a drug to a patient, the method comprising: determining pharmacokinetic parameters of the drug for a virtual patient population; determining disease predictive descriptors for the virtual patient population; assigning the pharmacokinetic parameters and disease predictive descriptors to the virtual patient population; processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and using the processed data to determine the characteristic of the HAE flare-up in response to administering the drug to the patient.

Some embodiments provide for a system, comprising: at least one computer-hardware processor; at least one non-transitory computer-readable hardware medium storing processor-executable instructions that, when executed by the at least one computer-hardware processor, cause the at least one computer-hardware processor to perform a method for determining a characteristic of a hereditary angioedema (HAE) flare-up in response to administering a drug to a patient, the method comprising: determining pharmacokinetic parameters of the drug for a virtual patient population; determining disease predictive descriptors for the virtual patient population; assigning the pharmacokinetic parameters and disease predictive descriptors to the virtual patient population; processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and using the processed data to determine the characteristic of the HAE flare-up in response to administering the drug to the patient.

Some embodiments provide for at least one non-transitory computer-readable hardware medium storing processor-executable instructions that, when executed by at least one computer-hardware processor, cause the at least one computer-hardware processor to perform a method for determining a characteristic of a hereditary angioedema (HAE) flare-up in response to administering a drug to a patient, the method comprising: determining pharmacokinetic parameters of the drug for a virtual patient population; determining disease predictive descriptors for the virtual patient population; assigning the pharmacokinetic parameters and disease predictive descriptors to the virtual patient population; processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and using the processed data to determine the characteristic of the HAE flare-up in response to administering the drug to the patient.

Some embodiments provide for a method for developing a virtual patient population comprising a plurality of virtual patients for input into a quantitative systems pharmacology (QSP) model of hereditary angioedema (HAE), wherein the QSP model is configured to represent autoactivation of Factor XII by elevating levels of FXIIa in response to an indication that a trigger has been input into the QSP model and to output an amount of one or more contact system proteins, the method comprising: assigning pharmacokinetic parameters to the virtual patient population; determining a baseline attack frequency and baseline attack severity for each patient in the virtual patient population; and assigning the baseline attack frequency and baseline attack severity to each patient in the virtual patient population.

Some embodiments provide for a system, comprising: at least one computer-hardware processor; at least one non-transitory computer-readable storage medium storing processor-executable instructions that, when executed by the at least one computer-hardware processor, cause the at least one computer-hardware processor to perform a method for developing a virtual population for input into a quantitative systems pharmacology (QSP) model of hereditary angioedema (HAE), wherein the QSP model is configured to represent autoactivation of Factor XII by elevating levels of FXIIa in response to an indication that a trigger has been input into the QSP model to output an amount of one or more contact system proteins, the method comprising: assigning pharmacokinetic parameters to the virtual patient population; determining a baseline attack frequency and a baseline attack severity for each patient in the virtual patient population; and assigning the baseline attack frequency and baseline attack severity to each patient in the virtual patient population.

Some embodiments provide for at least one non-transitory computer-readable storage medium storing processor-executable instructions that, when executed by at least one computer-hardware processor, cause the at least one computer-hardware processor to perform a method for developing a virtual population for input into a quantitative systems pharmacology (QSP) model of hereditary angioedema (HAE), wherein the QSP model is configured to represent autoactivation of Factor XII by elevating levels of FXIIa in response to an indication that a trigger has been input into the QSP model to output an amount of one or more contact system proteins, the method comprising: assigning pharmacokinetic parameters to the virtual patient population; determining a baseline attack frequency and a baseline attack severity for each patient in the virtual patient population; and assigning the baseline attack frequency and baseline attack severity to each patient in the virtual patient population.

### BRIEF DESCRIPTION OF DRAWINGS

Various aspects and embodiments of the application will be described with reference to the following figures. It should be appreciated that the figures are not necessarily drawn to scale. Items appearing in multiple figures are indicated by the same reference number in all the figures in which they appear. For purposes of clarity, not every component may be labeled in every drawing.

FIG. 1 illustrates a biological process map for HAE, in accordance with some embodiments of the technology described herein.

FIG. 2 illustrates an overview of an example model for modeling, simulating, and treating HAE, in accordance with some embodiments of the technology described herein.

FIG. 3 illustrates an example PK model, in accordance with some embodiments of the technology described herein.

FIG. 4 illustrates an example contact activation system PD model, in accordance with some embodiments of the technology described herein.

FIG. 5 illustrates an example in vitro assay procedure used in forming a fluorogenic assay PD model, in accordance with some embodiments of the technology described herein.

FIG. 6 illustrates an example illustration of protein level changes in HAE patients during an acute attack, in accordance with some embodiments of the technology described herein.

FIG. 7 illustrates example clinical samples of time intervals between acute attacks in HAE patients, in accordance with some embodiments of the technology described herein.

FIG. 8A illustrates an example representation of HAE virtual patient population capturing patient variability in pharmacokinetic parameters and propensity for acute attack represented by frequency (f) and severity (S), in accordance with some embodiments of the technology described herein.

FIG. 8B illustrates a method for developing a virtual patient population comprising a plurality of virtual patients to simulate HAE, in accordance with some embodiments of the technology described herein.

FIGS. 9A-9B illustrate example models of a trigger for an acute attack leading to auto-activation of the kinin-kallikrein pathway and production of elevated levels of bradykinin, in accordance with some embodiments of the technology described herein.

FIG. 10 illustrates an example representation of different phases of an acute attack as indicated by a reported pain score in untreated HAE patients, in accordance with some embodiments of the technology described herein.

FIGS. 11A-11C illustrate examples of acute attack modeling in a virtual population, in accordance with some embodiments of the technology described herein.

FIGS. 12A-12B illustrate examples of simulated PK profiles using the example PK model of FIG. 3, in accordance with some embodiments of the technology described herein.

FIG. 13 illustrates examples of simulated PK profiles using an example one-compartment PK model, in accordance with some embodiments of the technology described herein.

FIGS. 14A-15 illustrate examples of simulation output using the PD model of FIG. 5 representing the fluorescence assay compared with clinical data of measured level of kallikrein inhibition activity, in accordance with some embodiments of the technology described herein.

FIG. 16 illustrates dose-dependent inhibition of kallikrein by lanadelumab for a range of prekallikrein levers (250-650 nM) reported in the literature, in accordance with some embodiments of the technology described herein.

FIGS. 17A-17C illustrate comparisons of steady-state levels of proteins of the HAE contact system reported in literature and predicted levels using the contact activation system PD model of FIG. 2, in accordance with some embodiments of the technology described herein.

FIGS. 18A-18C illustrate example comparisons of bradykinin and factor XIIa levels in clinical data and predicted data using the PD model of FIG. 2, in accordance with some embodiments of the technology described herein.

FIGS. 19A-19C illustrates examples comparisons of cHMWK levels in clinical data from HAE patients under acute attack and predicted data using the PD model of FIG. 2, in accordance with some embodiments of the technology described herein.

FIG. 20 illustrates, schematically, an illustrative computing device for implementing aspects of the present disclosure, in accordance with some embodiments of the technology described herein.

FIG. 21 illustrates comparisons of cHMWK levels from clinical data to simulation output from the contact activation system PD model of FIG. 2 in HAE patients treated with different dosages of lanadelumab, in accordance with some embodiments of the technology described herein.

FIG. 22 illustrates comparisons of cHMWK levels from clinical data to simulation output from the acute attack PD model of FIG. 2 in HAE patients treated with different dosages of lanadelumab, in accordance with some embodiments of the technology described herein.

FIG. 23 illustrates comparisons of HAE acute attack rates from clinical data to simulation output from the acute attack PD model for HAE patients treated with different dosages of lanadelumab, in accordance with some embodiments of the technology described herein.

FIGS. 24A-24B illustrate example time profiles of bradykinin levels and BDKR-B2 receptor occupancy for virtual patients being treated with lanadelumab, in accordance with some embodiments of the technology described herein.

FIGS. 25A-25B illustrate example relationships between monthly attack rates and attack severity in a virtual patient population being treated with lanadelumab, in accordance with some embodiments of the technology described herein.

FIGS. 26A-26B illustrates example relationships between monthly attack rates and attack frequency in a virtual patient population being treated with lanadelumab, in accordance with some embodiments of the technology described herein.

FIG. 27 illustrates an example relationship between monthly attack rates and binding affinity of lanadelumab to kallikrein, in accordance with some embodiments of the technology described herein.

FIG. 28 illustrates example relationships of observed bradykinin levels and system model parameters, in accordance with some embodiments of the technology described herein.

FIG. 29 is a flow chart illustrating a computer implemented system and method for modeling, simulating, and evaluating treatments for HAE, in accordance with some embodiments of the technology described herein.

FIG. 30 illustrates an example method for modeling and simulating HAE, in accordance with some embodiments of the technology described herein.

FIG. 31 illustrates an example method for estimating one or more characteristics of a contact system in a patient in response to a trigger, in accordance with some embodiments of the technology described herein.

FIG. 32 illustrates an example method for determining a relationship between HAE attack frequency and a trigger rate for autoactivation of Factor XII, in accordance with some embodiments of the technology described herein.

FIG. 33 illustrates an example method for determining an effectiveness of an administered drug in treating HAE, in accordance with some embodiments of the technology described herein.

FIG. 34 illustrates a method for determining a characteristic of an HAE flare-up in response to administering a drug to a patient, in accordance with some embodiments of the technology described herein.

FIG. 35 illustrates an example method for determining an amount of a protein of a contact system in a patient in response to administration of a drug for treating HAE, in accordance with some embodiments of the technology described herein.

FIGS. 36A-36C illustrate example relationships between drug effectiveness in treating HAE and binding affinity, and half-life, in accordance with some embodiments of the technology described herein.

FIG. 37 illustrates an example relationship of monthly attack rates and inhibitions constants of administered drugs, in accordance with some embodiments of the technology described herein.

FIG. 38 illustrates an example method for determining a temporal profile illustrating an effect of a drug on a contact system in a patient, in accordance with some embodiments of the technology described herein.

FIG. 39 illustrates an example method for determining an effectiveness of a dosage of an administered drug in treating HAE, in accordance with some embodiments of the technology described herein.

FIG. 40 illustrates example relationships of drug exposure and HAE attack response, in accordance with some embodiments of the technology described herein.

FIG. 41 illustrates an example relationship drug exposure and HAE attack response, in accordance with some embodiments of the technology described herein.

FIG. 42 illustrates an example method for determining an effect of non-adherence to a dosing regimen of an administered drug in treating HAE, in accordance with some embodiments of the technology described herein.

FIG. 43A illustrates an example relationship between nonadherence to a dosage regimen and bradykinin levels, in accordance with some embodiments of the technology described herein.

FIG. 43B illustrates examples relationships between nonadherence rates and attack frequency, in accordance with some embodiments of the technology described herein.

### DETAILED DESCRIPTION

### Introduction

Aspects of the present application provide for methods and apparatuses for modeling, simulation, and treating hereditary angioedema. In particular, aspects of the present application provide for a quantitative systems pharmacology (QSP) model for modeling, simulating, and treating hereditary angioedema (HAE). In some embodiments, the QSP model may be configured to model HAE using FXII autoactivation as a trigger. For example, the QSP model may be configured to represent autoactivation of Factor XII by elevating levels of FXIIa in response to an indication that a trigger has been input into the QSP model. In some embodiments, the QSP model may be applied to evaluate new and existing treatment modalities for treating HAE.

In some embodiments, use of the QSP model described in the present application may provide various types of information about the contact system in a patient which would be impractical or impossible to clinically obtain. For example, the QSP model may provide, as output, levels of proteins of the contact system (e.g., bradykinin, cHMWK, plasma kallikrein, FXIIa, etc.), HAE acute attack frequency, severity and duration, among other types of information. In some embodiments, the QSP model may be implemented with a virtual population to execute a virtual clinical trial to evaluate the effects of a therapeutic intervention on HAE. In such embodiments, an attribute of the therapeutic intervention (e.g., half-life, binding affinity, dose, dose frequency, dose regimen, nonadherence percentage) may be correlated with an output of the QSP model to determine the effectiveness of the therapeutic intervention. The inventors have recognized that such techniques may facilitate development of new and more effective treatment modalities within the HAE field.

Overview of Hereditary Angioedema

According to some aspects of the present application, the apparatuses and methods described herein may be used to model, simulate and treat hereditary angioedema (HAE), also known as “Quincke edema,” C1 esterase inhibitor deficiency, C1 inhibitor deficiency, and formerly known as hereditary angioneurotic edema (HANE). HAE is characterized by unpredictable, recurrent attacks of severe subcutaneous or submucosal swelling (angioedema), which can affect, one or more parts of the body (e.g., the limbs, face, genitals, gastrointestinal tract, and airway). (Zuraw, 2008). Symptoms of HAE may include, for example, swelling in the arms, legs, lips, eyes, tongue, and/or throat, airway blockage that can involve throat swelling, sudden hoarseness and/or cause death from asphyxiation. (Bork et al., 2012; Bork et al., 2000). Approximately 50% of all HAE patients will experience a laryngeal attack in their lifetime, and there is no way to predict which patients are at risk of a laryngeal attack. (Bork et al., 2003; Bork et al., 2006). HAE symptoms may also include repeat episodes of abdominal cramping without obvious cause, and/or swelling of the intestines, which can be severe and can lead to abdominal cramping, vomiting, dehydration, diarrhea, pain, shock, and/or intestinal symptoms resembling abdominal emergencies, which may lead to unnecessary surgery. (Zuraw, 2008). Swelling may last up to five or more days. Most patients suffer multiple attacks per year. Swelling of the airway may be life threatening and cause death in some patients. Mortality rates for HAE are estimated at 15-33%, and HAE leads to about 15,000-30,000 emergency department visits per year.

HAE is an orphan disorder, the exact prevalence of which is unknown, but current estimates range from 1 per 10,000 to 1 per 150,000 persons, with many authors agreeing that 1 per 50,000 is likely the closest estimate. (Bygum, 2009; Goring et al., 1998; Lei et al., 2011; Nordenfelt et al., 2014; Roche et al., 2005). HAE is inherited in an autosomal dominant pattern, such that an affected person can inherit the mutation from one affected parent. New mutations in the gene can also occur, and thus HAE may occur in people with no history of the disorder in their family. It is estimated that 20-25% of cases result from a new spontaneous mutation.

Like adults, children with HAE can suffer from recurrent and debilitating attacks. Symptoms may present first appear in childhood, including very early in childhood with upper airway angioedema has been reported in HAE patients as young as the age of 3, and worsen during puberty. (Bork et al., 2003). In one case study of 49 pediatric HAE patients, 23 had suffered at least one episode of airway angioedema by the age of 18 (Farkas, 2010). An important unmet medical need exists among children with HAE, especially adolescents, since the disease commonly worsens after puberty (Bennett and Craig, 2015; Zuraw, 2008).

There are three types of HAE, known as types I, II, and III, with types I and II being able to be modeled, simulated, and treated by the techniques described herein, in some embodiments. It is estimated that HAE affects 1 in 50,000 people, that type I accounts for about 85 percent of cases, and that type II accounts for about 15 percent of cases, with type III being very rare.

Mutations in the SERPING1 gene cause hereditary angioedema type I and type II. The SERPING1 gene provides instructions for making the C1 inhibitor protein (also referred to as the C1-INH protein), which is important for controlling inflammation. C1 inhibitor blocks the activity of certain proteins, including generation of plasma kallikrein, that promote inflammation. Mutations that cause hereditary angioedema type I lead to reduced levels of C1 inhibitor in the blood. In contrast, mutations that cause type II result in the production of a C1 inhibitor that functions abnormally. Approximately 85% of patients have Type I HAE, characterized by very low production of functionally normal C1-INH protein, while the remaining approximately 15% of patients have Type II HAE and produce normal or elevated levels of a functionally impaired C1-INH (Zuraw, 2008).

Without the proper levels of functional C1 inhibitor to control the activation of the kinin-kallikrein cascade of the contact activation system, excessive amounts of bradykinin are generated from high molecular weight kininogen (HMWK), and there is increased vascular leakage mediated by bradykinin binding to the B2 receptor (B2-R) on the surface of endothelial cells (Zuraw, 2008). Bradykinin promotes inflammation by increasing the leakage of fluid through the walls of blood vessels into body tissues. Excessive accumulation of fluids in body tissues causes the episodes of swelling seen in individuals with HAE type I and type II.

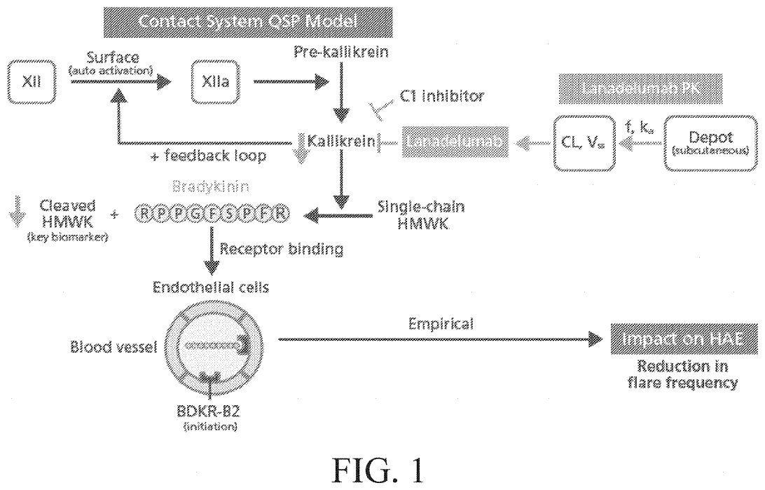

In particular, FIG. 1 illustrates a biological process map for HAE, in accordance with some embodiments of the technology described herein. Portions of the QSP model are further labeled on the biological process map and will be described further herein. As described herein, HAE is caused by deficiencies in controlling the contact activation system. Central to the contact system is the kinin-kallikrein cascade. When Factor XII is autoactivated, for example, due to one or more triggers, as described herein, FXII is converted into its activated form FXIIa. The activation of FXII cleaves pre-kallikrein to plasma kallikrein, which in turn cleaves single-chain High Molecular Weight Kininogen (HMWK). The cleaving of single-chain HMWK results in cleaved High Molecular Weight Kininogen (cHMWK) and liberation of potent pro-edema peptide Bradykinin. Bradykinin binds to its receptors (BDKR-B2) on the surface of endothelial cells, signaling cytoskeletal rearrangements and separation of cell-cell junctions culminating in fluid entry intro tissues (edema). In a healthy individual, the kinin-kallikrein cascade is kept in balance by plasma C1-INH, which binds to and inhibits both Factor XIIa and kallikrein, preventing aberrant pathway activation. However, in the case of individuals with HAE, endogenous C1-INH levels are insufficient or the C1-INH has aberrant protease inhibitor function, and activation of the contact system may be frequent and severe (referred to herein as an acute attack).

Trauma or stress, for example, dental procedures, sickness (e.g., viral illnesses such as colds and the flu), menstruation, and surgery can trigger an attack of angioedema. To prevent acute attacks of HAE, patients can attempt to avoid specific stimuli that have previously caused attacks. Doing so may constitute a significant interruption to a patient's daily life, and, in many cases, regardless of a patient's actions, an attack may occur without a known trigger. On average, untreated individuals have an attack every 1 to 2 weeks, and most episodes last for about 3 to 4 days. (ghr.nlm.nih.gov/condition/hereditary-angioedema). The frequency and duration of attacks may vary greatly among people with hereditary angioedema, even among people in the same family.

There currently exist a number of treatment modalities for HAE. Some treatment modalities for HAE can stimulate the synthesis of C1 inhibitor, or reduce C1 inhibitor consumption. Androgen medications, such as danazol, can reduce the frequency and severity of attacks by stimulating production of C1 inhibitor. Newer treatments attack the contact cascade. Ecallantide (KALBITOR®, DX-88, Dyax) inhibits plasma kallikrein and has been approved in the U.S. Icatibant (FIRAZYR®, Shire) inhibits the bradykinin B2 receptor, and has been approved in Europe and the U.S. Some treatment modalities, including Lanadelumab (Takhzyro or SHP643), a fully human IgG1 recombinant monoclonal antibody inhibitor of activated plasma kallikrein, treat and/or aim to prevent HAE or a symptom thereof by administering an antibody to a subject having or suspected of having HAE, for example, as described in PCT App. No. PCT/US2016/065980 titled “PLASMA KALLIKREIN INHIBITORS AND USES THEREOF FOR TREATING HEREDITARY ANGIOEDEMA ATTACK” filed Dec. 6, 2019 under Attorney Docket No. D0617.70110WO00, which is hereby incorporated by reference in its entirety herein. In such treatments, antibodies are used to inhibit an activity (e.g., inhibit at least one activity of plasma kallikrein, e.g., reduce Factor XIIa and/or bradykinin production) of plasma kallikrein, e.g., in vivo. The binding proteins can be used by themselves or conjugated to an agent, e.g., a cytotoxic drug, cytotoxin enzyme, or radioisotope. A summary of existing treatment modalities for HAE is given in Table 1 below.

TABLE 1
Summary of HAE Therapeutic Modalities

| Product | Target | Modality | Administration |
|---|---|---|---|
| C1-Inh (Cinryze) | Kallikrein & FXIIa | Protein | Prophylactic |
| Lanadelumab | Kallikrein | Antibody | Prophylactic |
| Kalbitor | Kallikrein | Recombinant peptide | Acute |
| Firazyr | BDKR-B2 | Synthetic peptide mimetic | Acute |

According to some aspects of the technology described herein, a QSP model is provided and used in computer-implemented methods for determining the effectiveness of therapeutic intervention in treating HAE, for example, determining an effect of an administered drug on the kinin-kallkrein cascade of the contact activation system. As shown in FIG. 1, in some embodiments, pharmacokinetic parameters for a drug, such as lanadelumab, may be input into the QSP model to determine an impact of the drug on HAE (e.g., by evaluating a reduction in HAE flare frequency).

Quantitative Systems Pharmacology Model Development

In order to better understand HAE and potential treatment modalities for HAE, the inventors have developed a quantitative systems pharmacology (QSP) model for modeling, simulating, and treating HAE. According to some embodiments, the QSP model is parameterized and verified with biological data in literature as well clinical data from one or more clinical trials.

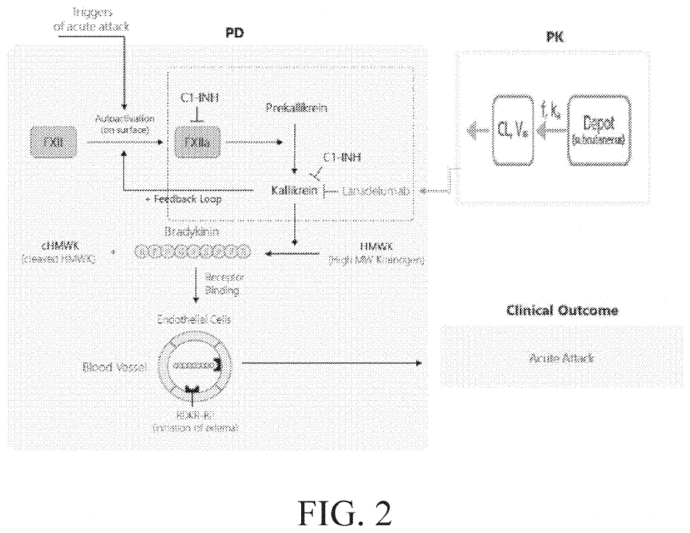

FIG. 2 illustrates an overview of an example model for modeling, simulating, and treating HAE, in accordance with some embodiments of the technology described herein. As shown in FIG. 2, the QSP model may include multiple individual models, including a pharmacokinetic (PK) model and one or more pharmacodynamics (PD) models. The PK model may provide PK parameters for use in the one or more PD models, for example, describing how characteristics of a patient (e.g., height, weight, gender, age, etc.) affect a drug administered to the patient (for example, affecting the concentration of the drug in the patient's bloodstream). The one or more PD models may illustrate a portion of the contact system in which HAE is triggered, including the kinin-kallikrein cascade, as described herein. In the illustrated embodiment, the one or more PD models comprise a fluorogenic assay PD model for modeling the inhibition of kallikrein by a therapeutic intervention and a contact activation system PD model for modeling the entire kinin-kallikrein cascade, as will be further described herein.

The QSP model shown in FIG. 2 further includes an acute attack model integrated with the contact activation system PD model for describing the trigger for an acute attack (also referred to herein as an HAE flare or flare-up). Measured clinical outcomes may include edema, pain, and acute attack. The PD model(s) may provide output for predicting acute attack frequency and severity, among other characteristics.

In some embodiments, the QSP model may be configured to model HAE using FXII autoactivation as a trigger. For example, as described herein, an HAE flare-up may occur at any time according to a number of triggers. In some cases, the cause of the HAE flare-up may be unknown and not directly related to a particular trigger. Thus, in some embodiments, the QSP model may be configured to represent autoactivation of Factor XII by elevating levels of FXIIa in response to an indication that a trigger has been input into the QSP model, without analyzing what the particular trigger is. In this manner, the heterogeneity of different flare-up triggers may be bypassed by the QSP model.

In some embodiments, the QSP model is utilized in computer-implemented methods for modeling, simulating, and treating HAE. For example, the various PK and PD models described herein may be used to evaluate the effectiveness of a new or existing treatment modality for HAE. In some embodiments, only some of the individual models may be utilized when implementing the QSP model in a computer-implemented method. For example, in some embodiments, the QSP model may be implemented without using the PK model(s) to better understand a response of the contact system in response to a trigger and in the absence of any therapeutic intervention. Therefore, as used herein, the quantitative systems pharmacology (QSP) model should be understood to encompass any combination of the PK and PD models described herein.

PK Model

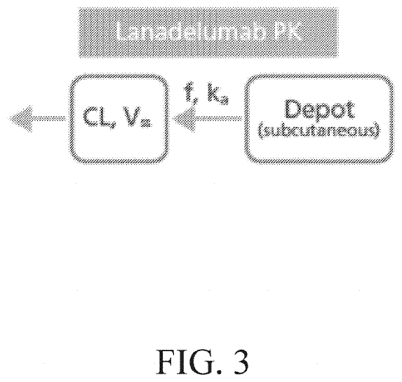

According to some aspects, the QSP model includes a PK model for providing PK parameters to the PD model. FIG. 3 illustrates an example PK model, in accordance with some embodiments of the technology described herein. The PK model may describe how a drug is absorbed and distributed by a particular patient, more particularly, the rate and extent of the distribution of the drug to different tissues and the rate of elimination of the drug. The PK model may be modeled as a series of differential equations describing the transit of the drug throughout the body.

As shown in FIG. 3, the PK model is a single-compartment PK model with a subcutaneous (SC) depot. In some embodiments, a non-compartmental PK model may be used. In some embodiments, the PK model may be a two-compartment PK model with a SC depot. In particular, the PK model may be divided into a central and peripheral compartment. The central compartment consists of plasma and tissues where distribution of the drug occurs more rapidly, whereas the peripheral compartment consists of tissues and plasma where the distribution of the drug occurs more slowly. The inventors have appreciated that use of a PK model having multiple compartments may account for non-homogeneities in the distribution of the drug.

The PK model may be used to model the PK behavior of a drug in a patient. For example, in some embodiments, the PK model is used to model the PK behavior of existing treatment modalities, such as lanadelumab. In some embodiments, the PK model may be used to model the PK behavior of a new and/or previously untested drug. For example, absorption rate (ka) and bioavailability (F) for a drug to be modeled may be input into the PK model and the predicted concentration of the drug in the patient may be output for inputting into the PD model.

PD Model(s)

According to some aspects, the QSP model comprises one or more PD models for modeling HAE. In the illustrated embodiment, the QSP model includes three individual PD models: (1) contact activation system PD model; (2) fluorogenic assay PD model; and (3) acute attack clinical outcome model. In the illustrated embodiment, the fluorogenic assay PD model is configured as a subset of contact activation system PD model and is used to estimate parameters (e.g., parameters relating to kallikrein inhibition) for parameterizing the QSP model. Thus, in some embodiments, the flourogenic assay PD model is used in the development of the QSP model, and the contact activation system PD model and acute attack clinical outcome model is used in applying the QSP model, as described herein. Table 2 gives a list of variables used in the PD models.

TABLE 2
List of variables in the PD models

| | |
|---|---|
| Unit | Description |

In Vascular Space

| | |
|---|---|
| nM | Degraded BK concentration |
| nM | BK concentration |
| nM | Degraded C1Inh concentration |
| nM | Degraded C1Inh_FXIIa concentration |
| nM | C1Inh_FXIIa concentration |
| nM | C1Inh concentration |
| nM | Degraded C1Inh_KAL concentration |
| nM | Degraded C1Inh_KAL_HMWK concentration |
| nM | Degraded C1Inh_KAL_HMWK concentration |
| nM | C1Inh_KAL concentration |
| nM | Degraded FXII concentration |
| nM | FXII concentration |
| nM | Degraded FXIIa concentration |
| nM | FXIIa concentration |
| nM | Degraded 2 Chain HMWK concentration |
| nM | HK2Chain concentration |
| nM | Degraded HMWK concentration |
| nM | HMWK concentration |
| nM | Degraded KAL concentration |
| nM | KAL_HK2Chain concentration |
| nM | KAL_HMWK concentration |
| nM | KAL concentration |
| nM | Lanadelumab concentration |
| nM | Lanadelumab_KAL_HMWK concentration |
| nM | Lanadelumab_KAL concentration |
| nM | Degraded preKAL concentration |
| nM | preKAL_HMWK concentration |
| nM | preKAL concentration |

In Proximal Space

| | |
|---|---|
| nM | BK concentration |
| number/cell | Number of BDKRB2 receptor |
| number/cell | Number of degraded surface BDKRB2 receptors |
| number/cell | Number of BK_BDKRB2 complex |
| number/cell | Number of degraded surface BK_BDKRB2 complex |
| nM | C1Inh concentration |
| nM | C1Inh_FXIIa concentration |
| number/cell | Number of C1Inh_FXIIa_gC1qR complex |
| number/cell | Number of degraded surface C1Inh_FXIIa_gC1qR complex |
| nM | C1Inh_KAL_HMWK concentration |
| number/cell | Number of C1Inh_KAL_HMWK_gC1qR complex |
| number/cell | Number of degraded surface C1Inh_KAL_HMWK_gC1qR complex |
| nM | FXII concentration |
| number/cell | Number of FXII_gC1qR complex |
| number/cell | Number of degraded surface FXII_gC1qR complex |
| nM | FXIIa concentration |
| number/cell | Number of FXIIa_gC1qR complex |
| number/cell | Number of degraded surface FXIIa_gC1qR complex |
| number/cell | Number of gC1qR receptor |
| number/cell | Number of degraded surface gC1qR receptors |
| nM | KAL_HK2Chain concentration |
| number/cell | Number of KAL_HK2Chain_gC1qR complex |
| number/cell | Number of degraded surface KAL_HK2Chain_gC1qR complex |
| nM | KAL_HMWK concentration |
| number/cell | Number of KAL_HMWK_gC1qR complex |
| number/cell | Number of degraded surface KAL_HMWK_gC1qR complex |
| nM | Lanadelumab concentration |
| nM | Lanadelumab_KAL_HMWK concentration |
| number/cell | Number of Lanadelumab_KAL_HMWK_gC1qR complex |
| number/cell | Number of degraded surface Lanadelumab_KAL_HMWK_gC1qR complex |
| nM | preKAL_HMWK concentration |
| number/cell | Number of preKAL_HMWK_gC1qR complex |
| number/cell | Number of degraded surface preKAL_HMWK_gC1qR complex |

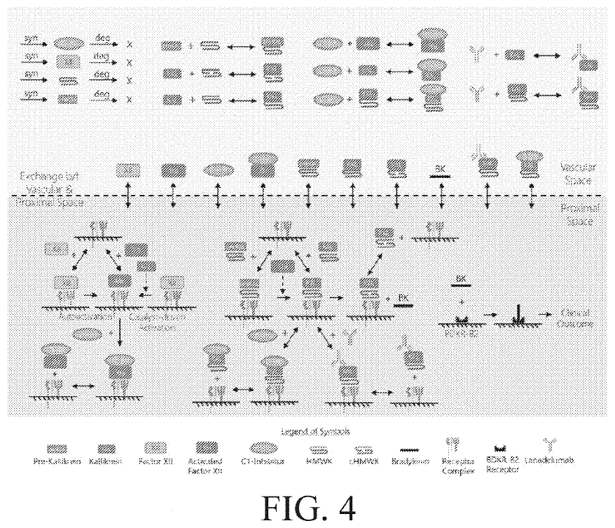

FIG. 4 illustrates an example contact activation system PD model, in accordance with some embodiments of the technology described herein. The contact activation system PD model describes the kinin-kallikrein cascade of the contact system involving contact factor proteins, FXII/FXIIa, preKAL/KAL (kallikrein) and HMWK (high molecular weight kininogen), activated on the endothelial cell surface to release the vasoactive peptide (bradykinin). The pathway is a cascade of activation and cleavage reactions involving these proteins and their complexes in plasma and about to receptor complexes on the epithelial cell surface. These reactions are illustrated in the example diagram in FIG. 4 and are listed in Table 3a below. The governing equations for all the proteins in the model are shown in Table 3b below.

TABLE 3a
List of reactions in model
In Vascular Space

| | |
|---|---|
| Binding/unbinding | C1Inh_in_plasma + FXIIa_in_plasma ↔ C1Inh_FXIIa_in_plasma |
| between C1Inh | |
| and FXIIa | |
| Binding/unbinding | C1Inh_in_plasma + KAL_in_plasma ↔ C1Inh_HMWK_in_plasma |
| between C1Inh | |
| and KAL | |
| Binding/unbinding | C1Inh_in_plasma + KAL_HMWK_in_plasma ↔ C1Inh_KAL_HMWK_in_plasma |
| between C1Inh | |
| and KAL_HMWK | |
| Binding/unbinding | KAL_in_plasma + HK2Chain_in_plasma ↔ KAL_HK2Chain_in_plasma |
| between KAL and | |
| HK2Chain | |
| Binding/unbinding | KAL_in_plasma + HMWK_in_plasma ↔ KAL_HMWK_in_plasma |
| between KAL and | |
| HMWK | |
| Binding/unbinding | KAL_in_plasma + Lanadelumab_in_plasma ↔ Lanadelumab_KAL_in_plasma |
| between KAL and | |
| Lanadelumab | |
| Binding/unbinding | KAL_HMWK_in_plasma + Lanadelumab_in_plasma ↔ Lanadelumab_KAL_HMWK_in_plasma |
| between | |
| KAL_HMWK and | |
| Lanadelumab | |
| Binding/unbinding | preKAL_in_plasma + HMWK_in_plasma ↔ preKAL_HMWK_in_plasma |
| between preKAL | |
| and HMWK | |
| Degradation of BK | BK_in_plasma → BK_degraded |
| Degradation of | C1Inh_in_plasma → C1Inh_degraded |
| C1Inh | |
| Degradation of | C1Inh_FXIIa_in_plasma → C1Inh_FXIIa_degraded |
| C1Inh_FXIIa | |
| Degradation of | C1Inh_KAL_in_plasma → C1Inh_KAL_degraded |
| C1Inh_KAL | |
| Degradation of | C1Inh_KAL_HMWK_in_plasma → C1Inh_KAL_HMWK_degraded |
| C1Inh_KAL_HMWK | |
| Degradation of | FXII_in_plasma → FXII_degraded |
| FXII | |
| Degradation of | FXIIa_in_plasma → FXIIa_degraded |
| FXIIa | |
| Degradation of | HK2Chain_in_plasma → HK2Chain_degraded |
| HK2Chain | |
| Degradation of | HMWK_in_plasma → HMWK_degraded |
| HMWK | |
| Degradation of | KAL_in_plasma → KAL_degraded |
| KAL | |
| Degradation of | preKAL_in_plasma → preKAL_degraded |
| preKAL | |
| Synthesis of C1Inh | → C1Inh_in_plasma |
| Synthesis of FXII | → FXII_in_plasma |
| Synthesis of | → HMWK_in_plasma |
| HMWK | |
| Synthesis of | → preKAL_in_plasma |
| preKAL | |

In Proximal Space

| | |
|---|---|
| Activation of | FXII_gC1qR → FXIIa_gC1qR |
| FXII_gC1qR with | |
| KAL as catalyst | |
| Activation of | preKAL_HMWK_gC1qR → KAL_HMWK_gC1qR |
| preKAL_HMWK_gC1qR | |
| Auto-activation of | FXII_gC1qR → FXIIa_gC1qR |
| FXII_gC1qR | |
| Binding between | BK + BDKRB2 → BK_BDKRB2 |
| BK and BDKRB2 | |
| Binding between | C1Inh + FXIIa_gC1qR → C1Inh_FXIIa_gC1qR |
| C1Inh and | |
| FXIIa_gC1qR | |
| Binding between | C1Inh + KAL_HMWK_gC1qR → C1Inh_KAL_HMWK_gC1qR |
| C1Inh and | |
| KAL_HMWK_gC1qR | |
| Binding/unbinding | C1Inh_FXIIa + gC1qR ↔ C1Inh_FXIIa_gC1qR |
| between | |
| C1Inh_FXIIa and | |
| gC1qR | |
| Binding/unbinding | C1Inh_KAL_HMWK + gC1qR ↔ C1Inh_KAL_HMWK_gC1qR |
| between | |
| C1Inh_KAL_HM | |
| WK and gC1qR | |
| Binding/unbinding | FXII + gC1qR ↔ FXII_gC1qR |
| between FXII and | |
| gC1qR | |
| Binding/unbinding | FXIIa + gC1qR ↔ FXIIa_gC1qR |
| between FXIIa and | |
| gC1qR | |
| Binding/unbinding | KAL_HK2Chain + gC1qR ↔ KAL_HK2Chain_gC1qR |
| between | |
| KAL_HK2Chain | |
| and gC1qR | |
| Binding/unbinding | KAL_HMWK + gC1qR ↔ KAL_HMWK_gC1qR |
| between | |
| KAL_HMWK and | |
| gC1qR | |
| Binding/unbinding | KAL_HMWK_gC1qR + Lanadelumab ↔ Lanadelumab_KAL_HMWK_gC1qR |
| between | |
| KAL_HMWK_gC1qR and | |
| Lanadelumab | |
| Binding/unbinding | Lanadelumab_KAL_HMWK + gC1qR ↔ Lanadelumab_KAL_HMWK_gC1qR |
| between | |
| Lanadelumab_KAL_HMWK | |
| and gC1qR | |
| Binding/unbinding | preKAL_HMWK + gC1qR ↔ preKAL_HMWK_gC1qR |
| between | |
| preKAL_HMWK | |
| and gC1qR | |
| Cleavage of | KAL_HMWK_gC1qR → KAL_HMWK_gC1qR + BK |
| KAL_HMWK_gC1qR | |
| Degradation of | BDKRB2 → BDKRB2_degraded |
| BDKRB2 | |
| Degradation of | BK_BDKRB2 → BK_BDKRB2_degraded |
| BK_BDKRB2 | |
| Degradation of | C1Inh_FXIIa_gC1qR → C1Inh_FXIIa_gC1qR_degraded |
| C1Inh_FXIIa_gC1qR | |
| Degradation of | C1Inh_KAL_HMWK_gC1qR → C1Inh_KAL_HMWK_gC1qR_degraded |
| C1Inh_KAL_HMWK_gC1qR | |
| Degradation of | FXII_gC1qR → FXII_gC1qR_degraded |
| FXII_gC1qR | |
| Degradation of | FXIIa_gC1qR → FXIIa_gC1qR_degraded |
| FXIIa_gC1qR | |
| Degradation of | gC1qR → gC1qR_degraded |
| gC1qR | |
| Degradation of | KAL_HK2Chain_gC1qR → KAL_HK2Chain_gC1qR_degraded |
| KAL_HK2Chain_gC1qR | |
| Degradation of | KAL_HMWK_gC1qR → KAL_HMWK_gC1qR_degraded |
| KAL_HMWK_gC1qR | |
| Degradation of | Lanadelumab_KAL_HMWK_gC1qR → Lanadelumab_KAL_HMWK_gC1qR_degraded |
| Lanadelumab_KAL_HMWK_gC1qR | (R49) |
| Degradation of | preKAL_HMWK_gC1qR → preKAL_HMWK_gC1qR_degraded |
| preKAL_HMWK_gC1qR | |
| Synthesis of | → BDKRB2 |
| BDKRB2 | |
| Synthesis of | → gC1qR |
| gC1qR | |

Exchange between Proximal and Vascular Space

| | |
|---|---|
| Exchange of BK | BK_in_plasma ↔ BK |
| Exchange of | C1Inh_in_plasma ↔ C1Inh |
| C1Inh | |
| Exchange of | C1Inh_FXIIa_in_plasma ↔ C1Inh_FXIIa |
| C1Inh_FXIIa | |
| Exchange of | C1Inh_KAL_HMWK_in_plasma ↔ C1Inh_KAL_HMWK |
| C1Inh_KAL_HMWK | |
| Exchange of FXII | FXII_in_plasma ↔ FXII |
| Exchange of FXIIa | FXIIa_in_plasma ↔ FXIIa |
| Exchange of | KAL_HK2Chain_in_plasma ↔ KAL_HK2Chain |
| KAL_HK2Chain | |
| Exchange of | KAL_HMWK_in_plasma ↔ KAL_HMWK |
| KAL_HMWK | |
| Exchange of | Lanadelumab_KAL_HMWK_in_plasma ↔ Lanadelumab_KAL_HMWK |
| Lanadelumab_KAL_HMWK | |
| Exchange of | preKAL_HMWK_in_plasma ↔ preKAL_HMWK |
| preKAL_HMWK | |

TABLE 3b
List of governing equations in model

| | In Vascular Space |
|---|---|
| 1 | V_{medium} · dBK_degraded/dt = V_{medium} · kdeg_{BK} · BK_in_plasma |
| 2 | V_{medium} · dBK_in_plasma/dt = −V_{medium} · kdeg_{BK} · BK_in_plasma − (k12_{BK} · |
| | BK_in_plasma · V_{medium} − k21_{BK} · BK · Vₚᵣₒₓᵢₘₐₗ |
| 3 | V_{medium} · dC1Inh_degraded/dt = V_{medium} · kdeg_{C1Inh} · C1Inh_in_plasma |
| 4 | V_{medium} · dC1Inh_FXIIa_degraded/dt = V_{medium} · kdeg_Bound_{C1Inh} · |
| | C1Inh_FXIIa_in_plasma |
| 5 | V_{medium} · dC1Inh_FXIIa_in_plasma/dt = V_{medium} · (kon_{C1Inh}_{_{—}}_{FXIIa} · |
| | C1Inh_in_plasma · FXIIa_in_plasma − koff_{C1Inh}_{_{—}}_{FXIIa} · |
| | C1Inh_FXIIa_in_plasma) − V_{medium} · kdeg_Bound_{C1Inh} · |
| | C1Inh_FXIIa_in_plasma − (k12 · C1Inh_FXIIa_in_plasma · V_{medium} − k21 · |
| | C1Inh_FXIIa · Vₚᵣₒₓᵢₘₐₗ) |
| 6 | V_{medium} · dC1Inh_in_plasma/dt = flux_C1Inh_inj_nmol_per_hr + V_{medium} · |
| | ksyn_{c1inh} − V_{medium} · kdeg_{C1inh} · C1Inh_in_plasma − V_{medium} · (kon_{C1Inh}_{_{—}}_{KAL} · |
| | C1Inh_in_plasma · KAL_in_plasma − koff_{C1Inh}_{_{—}}_{KAL} · |
| | C1Inh_KAL_in_plasma) − V_{medium} · (kon_{C1Inh}_{_{—}}_{KAL} · C1Inh_in_plasma · |
| | KAL_HMWK_in_plasma − koff_{C1Inh}_{_{—}}_{KAL} − C1Inh_KAL_HMWK_in_plasma) − |
| | V_{medium} · (kon_{C1Inh}_{_{—}}_{FXIIa} · C1Inh_in_plasma − FXIIa_in_plasma − koff_{C1Inh}_{_{—}}_{FXIIa} · |
| | C1Inh_FXIIa_in_plasma) − (k12 · C1Inh_in_plasma · V_{medium} − k21 · |
| | C1Inh · Vₚᵣₒₓᵢₘₐₗ) |
| 7 | V_{medium} · dC1Inh_KAL_degraded/dt = V_{medium} · kdeg_Bound_{C1Inh} · |
| | C1Inh_KAL_ in_plasma |
| 8 | V_{medium} · dC1Inh_KAL_HMWK_degraded/dt = V_{medium} · kdeg_Bound_{C1Inh} · |
| | C1Inh_KAL_HMWK_in_plasma |
| 9 | V_{medium} · dC1Inh_KAL_HMWK_in_plasma/dtdt = |
| | V_{medium} · (kon_{C1Inh}_{_{—}}_{KAL} · C1Inh_in_plasma · KAL_HMWK_in_plasma − |
| | koff_{C1Inh}_{_{—}}_{KAL} · C1Inh_KAL_HMWK_in_plasma) − V_{medium} · kdeg_Bound_{C1Inh} · |
| | C1Inh_KAL_HMWK_in_plasma − (k12 · C1Inh_KAL_HMWK_in_plasma · |
| | V_{medium} − k21 · C1Inh_KAL_HMWK · Vₚᵣₒₓᵢₘₐₗ) |
| 10 | V_{medium} · dC1InhKAL_in_plasma/dt = V_{medium} · (kon_{C1Inh}_{_{—}}_{KAL} · |
| | C1Inh_in_plasma · KAL_in_plasma − koff_{C1Inh}_{_{—}}_{KAL} · |
| | C1Inh_KAL_in_plasma) − V_{medium} · kdeg_Bound_{C1Inh} · |
| | C1Inh_KAL_ in_plasma |
| 11 | V_{medium} · dFXII_degraded/dt = V_{medium} · kdeg_{FXII} · FXII_in_plasma |
| 12 | V_{medium} · dFXII_in_plasma/dt = V_{medium} · ksyn_{FXII} · V_{medium} · kdeg_{FXII} · |
| | FXII_in_plasma − (k12 · FXII_in_plasma − V_{medium} − k21 · FXII · Vₚᵣₒₓᵢₘₐₗ) |
| 13 | V_{medium} · dFXIIa_degraded/dt = V_{medium} · kdeg_{FXIIa} · FXIIa_in_plasma |
| 14 | V_{medium} · dFXIIa_in_plasma/dt = −V_{medium} · kdeg_{FXIIa} · FXIIa_in_plasma − |
| | V_{medium} · (kon_{C1Inh}_{_{—}}_{FXIIa} · C1Inh_in_plasma_FXIIa_in_plasma − koff_{C1Inh}_{_{—}}_{FXIIa} · |
| | C1Inh_FXIIa_in_plasma) − (k12 · FXIIa_in_plasma · V_{medium} − k21 · FXIIa · Vₚᵣₒₓᵢₘₐₗ) |
| 15 | V_{medium} · dHK2Chain_in_plasma/dt = V_{medium} · kdegcHMWK · |
| | HK2Chain_in_plasma |
| 16 | V_{medium} · dHK2Chain_degraded/dt = −V_{medium} · (kon_{KAL}_{_{—}}_{HK2Chain} · |
| | KAL_in_plasma · HK2Chain_in_plasma − koff_{KAL}_{_{—}}_{HK2Chain} · |
| | KAL_HK2Chain_in_plasma) − V_{medium} · kdeg_{cHMWK} · HK2Chain_in_plasma |
| 17 | V_{medium} · dHMWK_degraded/dt = V_{medium} · kdeg_{HMWK} · HMWK_in_plasma |
| 18 | V_{medium} · dHMWK_in_plasma/dt = V_{medium} · ksyn_{HMWK} − V_{medium} · kdeg_{HMWK} · |
| | HMWK_in_plasma − V_{medium} (kon_{preKAL}_{_{—}}_{HMWK} · preKAL_in_plasma · |
| | HMWK_in_plasma − koff_{preKAL}_{_{—}}_{HMWK} · preKAL_HMWK_in_plasma) − |
| | V_{medium} · (kon_{KAL}_{_{—}}_{HMWK} · KAL_in_plasmaHMWK_in_plasma − koff_{KAL}_{_{—}}_{HMWK} · |
| | KAL_HMWK_in_plasma) |
| 19 | V_{medium} · dKAL_degraded/dt = V_{medium} · kdeg_{KAL} · KAL_in_plasma |
| 20 | V_{medium} · dKAL_HK2Chain_in_plasma/dt = −(k12 · |
| | KAL_HK2Chain_in_plasma · V_{medium} − k21 · KAL_HK2Chain · Vₚᵣₒₓᵢₘₐₗ) + |
| | V_{medium} · (kon_{KAL}_{_{—}}_{HK2Chain} · KAL_in_plasma · HK2Chain_in_plasma − |
| | koff_{KAL}_{_{—}}_{HK2Chain} · KAL_HK2Chain_in_plasma) |
| 21 | V_{medium} · dKAL_HMWK_in_plasma/dt = V_{medium} · (kon_{KAL}_{_{—}}_{HMWK} · |
| | KAL_in_plasma · HMWK_in_plasma − koff_{KAL}_{_{—}}_{HMWK} · |
| | KAL_HMWK_in_plasma) − V_{medium} · (kon_{C1Inh}_{_{—}}_{KAL} · C1Inh_in_plasma · |
| | KAL_HMWK_in_plasma − koff_{C1Inh}_{_{—}}_{KAL} · C1Inh_KAL_HMWK_in_plasma) − |
| | V_{medium} (kon_{KAL}_{_{—}}Lanadelumab · KAL_HMWK_in plasma · |
| | Lanadelumab_in_plasma − koff_{KAL}_{_{—}}_{Lanadelumab} · |
| | Lanadelumab_KAL_HMWK_in_plasma) − (k12 · KAL_HMWK_in_plasma · |
| | V_{medium} − k21 · KAL_HMWK · Vₚᵣₒₓᵢₘₐₗ) |
| 22 | V_{medium} · dKAL_in_plasma/dt = −V_{medium} · kdeg_{KAL} · KAL_in_plasma − |
| | V_{medium} · (kon_{KAL}_{_{—}}_{HMWK} · KAL_in_plasmaHMWK_in_plasma − koff_{KAL}_{_{—}}_{HMWK} · |
| | KAL_HMWK_in_plasma) − V_{medium} · (kon_{C1Inh}_{_{—}}_{KAL} · C1Inh_in_plasma · |
| | KAL_in_plasma − koff_{C1Inh}_{_{—}}_{KAL} · C1Inh_KAL_in_plasma) − V_{medium} |
| | (kon_{KAL}_{_{—}}_{HK2Chain} · KAL_in_plasma · HK2Chain_in_plasma − koff_{KAL}_{_{—}}_{HK2Chain} · |
| | KAL_HK2Chain_in_plasma) − V_{medium} · (kon_{KAL}_{_{—}}_{Lanadelumab} · |
| | KAL_in_plasmaLanadelumab_in_plasma − koff_{KAL}_{_{—}}_{Lanadelumab} · |
| | Lanadelumab_KAL_in_plasma) |
| 23 | V_{medium} · dLanadelumab_{——}KAL_HMWK_in_plasma/dt = V_{medium} · |
| | (kon_{KAL}_{_{—}}_{Lanadelumab} · KAL_HMWK_in_plasma · Lanadelumab_in_plasma − |
| | koff_{KAL}_{_{—}}_{Lanadelumab} · Lanadelumab_KAL_HMWK_in_plasma) − (k12 · |
| | Lanadelumab_KAL_HMWK_in_plasma · V_{medium} − k21 · |
| | Lanadelumab_KAL_HMWK · Vₚᵣₒₓᵢₘₐₗ) |
| 24 | V_{medium} · dLanadelumab_KAL_in_plasma/dt = V_{medium} · (kon_{KAL}_{_{—}}_{Lanadelumab} · |
| | KAL_in_plasma · Lanadelumab_in_plasma − koff_{KAL}_{_{—}}_{Lanadelumab} · |
| | Lanadelumab_KAL_in_plasma) |
| 25 | V_{medium} · dpreKAL_degraded/dt = V_{medium} · kdeg_{preKAL} · preKAL_in_plasma |
| 26 | V_{medium} · dpreKAL_HMWK_in_plasma/dt = =V_{medium} · (kon_{preKAL}_{_{—}}_{HMWK} · |
| | preKAL_in_plasma · HMWK_in_plasma − koff_{preKAL}_{_{—}}_{HMWK} · |
| | preKAL_HMWK_in_plasma) − (k12 · preKAL_HMWK_in_plasma · V_{medium} − |
| | k21 · preKAL_HMWK · Vₚᵣₒₓᵢₘₐₗ) |
| 27 | V_{medium} · dpreKAL_in_plasma/dt V_{medium} · ksyn_{preKAL} − V_{medium} · kdeg_{preKAL} · |
| | preKAL_in_plasma − V_{medium} (kon_{preKAL}_{_{—}}_{HMWK} · preKAL_in_plasma · |
| | HMWK_in_plasma − koff_{preKAL}_{_{—}}_{HMWK} · preKAL_HMWK_in_plasma) |

In Proximal Space

| | |
|---|---|
| 28 | Vₚᵣₒₓᵢₘₐₗ = dBK/dt = (k12_{BK} · BK_in_plasma · V_{medium} − k21_{BK} · BK · Vₚᵣₒₓᵢₘₐₗ) + |
| | (kcat_{HMWK}_{_{—}}_{cleavage} · KAL_HMWK_gC1qR − (kon_{BK}_{_{—}}_{BDKKB2} · BK · BDKBB2 − |
| | koff_{BK}_{_{—}}_{BDKRB2} · BK_BDKBB2)) Num_to_Conc_Converter · Vₚᵣₒₓᵢₘₐₗ |
| 29 | dBDKRB2/dt = ksyn_{BDKRB2} − kdeg_{BDKRB2} · BDKRB2 − (kon_{BK}_{_{—}}_{BDKRB2} · BK · |
| | BDKBB2 − koff_{BK}_{_{—}}_{BDKRB2} · BK_BDKBB2) |
| 30 | dBDKRB2_degraded/dt = kdeg_{BDKRB2} · BDKBB2 |
| 31 | dBK_BRKRB2/dt = (kon_{BK}_{_{—}}_{BDKRB2} · BK · BDKBB2 − koff_{BK}_{_{—}}_{BDKRB2} · |
| | BK_BBKBB2) − kdeg_{BDKRB2} · BK_BDKRB2 |
| 32 | dBK_BDKRB2_degraded/dt = kdeg_{BDKRB2} · BK_BDKBB2 |
| 33 | dC1Inh_FXIIa_gC1qR/dt = (kon_{C1Inh}_{_{—}}_{FXIIa} · C1Inh · FXIIa_gC1qR − |
| | koff_{C1Inh}_{_{—}}_{FXIIa} · C1Inh_FXIIa_gC1qR) + (kon_{FXIIa}_{_{—}}_{gC1qR} · C1Inh_FXIIa · |
| | gC1qR − koff_{FXIIa}_{_{—}}_{gC1qR} · C1Inh_FXIIa_gC1qR) − kdeg_{gC1qR} · |
| | C1Inh_FXIIa_gC1qR |
| 34 | dC1Inh_FXIIa_gC1qR_degraded/dt = kdeg_{gC1qR} · C1Inh_FXIIa_gC1qR |
| 35 | dC1Inh_KAL_HMWK_gC1qR/dt = (kon_{C1Inh}_{_{—}}_{KAL} · C1Inh · |
| | KAL_HMWK_gC1qR − koff_{C1Inh}_{_{—}}_{KAL} · C1Inh_KAL_HMWK_gC1qR) + |
| | (kon_{HMWK}_{_{—}}_{gC1qR} · C1Inh_KAL_HMWK · gC1qR − koff_{HMWK}_{_{—}}_{gC1qR} · |
| | C1Inh_KAL_HMWK_gC1qR) − kdeg_{gC1qR} · C1Inh_KAL_HMWK_gC1qR |
| 36 | dC1Inh_KAL_HMWK_gC1qR_degraded/dt = kdegC1qR · |
| | C1Inh_KAL_HMWK_gC1qR |
| 37 | dFXII_gC1qR/dt = (kon_{FXII}_{_{—}}_{gC1qR} · FXII · gC1qR − koff_{FXII}_{_{—}}_{gC1qR} · |
| | FXII_gC1qR) − Fold_increase_{FXII}_{_{—}}_{AutoActication} · kcat_{FXII}_{_{—}}_{AutoActivation} · FXII_gC1qR − |
| | kcat_{FXII}_{_{—}}_{AutoActivation} · KAL_HMWK_gC1qRFXII_gC1qR · |
| | Num_to_Conc_converter/(km_{FXII}_{_{—}}_{AutoActivation} + FXII_gC1qR · |
| | Num_to_Conc_converter) − kdeg_{gC1qR} · FXII_gC1qR |
| 38 | dFXII_gC1qR_degraded/dt = kdeg_{gC1qR} · FXII_gC1qR |
| 39 | dFXIIa_gC1qR/dt = Fold_increase_{FXII}_{_{—}}_{AutoActivation} · kcat_{FXII}_{_{—}}_{AutoActivation} · |
| | FXII_gC1qR + kcat_{FXII}_{_{—}}_{AutoActivation} · KAL_HMWK_gC1qR · FXII_gC1qR · |
| | Num_to_Conc_converter/(km_{FXII}_{_{—}}_{AutoActivation} + FXII_gC1qR · |
| | Num_to_Conc_converter) + (konFXIIa_gC1qR · FXIIa · gC1qR − |
| | koff_{FXIIa}_{_{—}}_{gC1qR} · FXIIa_gC1qR) − (kon_{C1Inh}_{_{—}}_{FXIIa} · C1Inh · FXIIa_gC1qR − |
| | koff_{C1Inh}_{_{—}}_{FXIIa} · C1InhFXIIa_gC1qR) − kdeg_{gC1qR} · FXIIa_gC1qR |
| 40 | dFXIIa_gC1qR_degraded/dt = =kdeg_{gC1qR} · FXIIa_gC1qR |
| 41 | dgC1qR/dt = =ksyn_{gC1qR} − kdeg_{gC1qR} · gC1qR − (kon_{FXII}_{_{—}}_{gC1qR} · FXII · gC1qR − |
| | koff_{FXII}_{_{—}}_{gC1qR} · FXII_gC1qR) − (kon_{HMWK}_{_{—}}_{gC1qR} · preKAL_HMWK · gC1qR − |
| | koff_{HMWK}_{_{—}}_{gC1qR} · preKAL_HMWK_gC1qR) − (kon_{FXIIa}_{_{—}}_{gC1qR} · FXIIa · gC1qR − |
| | koff_{FXIIa}_{_{—}}_{gC1qR} · FXIIa_gC1qR) − (kon_{HMWK}_{_{—}}_{gC1qR} · preKAL_HMWK · gC1qR − |
| | koff_{HMWK}_{_{—}}_{gC1qR} · KAL_HMWK_gC1qR) − (kon_{HK2Chain}_{_{—}}_{gC1qR} · KAL_HK2Chain · |
| | gC1qR − koff_{HK2Chain}_{_{—}}_{gC1qR} · KAL_HK2Chain_gC1qR) − (kon_{FXIIa}_{_{—}}_{gC1qR} · |
| | C1Inh_FXIIa · gC1qR − koff_{FXIIa}_{_{—}}_{gC1qR} · C1Inh_FXIIa_gC1qR) − |
| | (kon_{HMWK}_{_{—}}_{gC1qR} · C1Inh_KAL_HMWK · gC1qR − koff_{HMWK}_{_{—}}_{gC1qR} · |
| | C1Inh_KAL_HMWK_gC1qR) − (kon_{HMWK}_{_{—}}_{gC1qR} · Lanadelumab_KAL_HMWK · |
| | gC1qR − koff_{HMWK}_{_{—}}_{gC1qR} · Lanadelumab_KAL_HMWK_gC1qR) |
| 42 | dgC1qR_degraded/dt = kdeg_{gC1qR} · gC1qR |
| 43 | dKAL_HK2Chain_gC1qR/dt = kcat_{HMWK}_{_{—}}_{cleavage} · KAL_HMWK_gC1qR + |
| | (kon_{HK2Chain}_{_{—}}_{gC1qR} · KAL_HK2Chain · gC1qR − koff_{HK2Chain}_{_{—}}_{gC1qR} · |
| | KAL_HK2Chain_gC1qR) − kdeg_{gC1qR} · KAL_HK2Chain · gC1qR |
| 44 | dKAL_HK2Chain_gC1qR_degraded/dt = kdeg_{gC1qR} · |
| | KAL_HK2Chain_gC1qR |
| 45 | dKAL_HMWK_gC1qR/dt = kcat_{preKAL}_{_{—}}_{Activation} · FXIIa_gC1qR · |
| | preKAL_HMWK_gC1gR · Num_to_Conc_converter/(km_{preKAL}_{_{—}}_{Activation} + |
| | preKAL_HMWK_gC1qR · Num_to_conc_converter) − kcat_{HMWK}_{_{—}}_{cleavage} · |
| | KAL_HMWK_gC1qR + (kon_{HMWK}_{_{—}}_{gC1qR} · KAL_HMWK · gC1qR − |
| | k_{offHMWK}_{_{—}}_{gC1qR} · KAL_HMWK_gC1qR) − (kon_{C1nh}_{_{—}}_{KAL} · |
| | C1InhKAL_HMWK_gC1qR − koff_{C1Inh}_{_{—}}_{KAL} · C1Inh_KAL_HMWK_gC1qR) − |
| | kdeg_{gC1qR} · KAL_HMWK_gC1qR − (kon_{KAL}_{_{—}}_{Lanadelumab} − KAL_HMWK_gC1qR · |
| | Lanadelumab − koff_{KAL}_{_{—}}_{Lanadelumab} · Lanadelumab_KAL_HMWK_gC1qR) |
| 46 | dKAL_HMWK_gC1qR_degraded/dt = kdeg_{gC1qR}· KAL_HMWK_gC1qR |
| 47 | dLanadelumab_KAL_HMWK_gC1qR/dt = (kon_{KAL}_{_{—}}_{Lanadelumab} · |
| | KAL_HMWK_gC1qR · Lanadelumab − koff_{KAL}_{_{—}}_{Lanadelumab} · |
| | Lanadelumab_KAL_HMWK_gC1qR) + (kon_{HMWK}_{_{—}}_{gC1qR} · |
| | Lanadelumab_KAL_HMWKgC1qR · koff_{HMWK}_{_{—}}_{gC1qR} · |
| | Lanadelumab_KAL_HMWK_gC1qR) − kdeg_{Lanadelumab}_{_{—}}_{KAL}_{_{—}}_{HMWK}_{_{—}}_{gC1qR} · |
| | Lanadelumab_KAL_HMWK_gC1qR |
| 48 | dLanadelumab_KAL_HMWK_gC1qR_degraded/dt = |
| | kdeg_{Lanadelumab}_{_{—}}_{KAL}_{_{—}}_{HMWK}_{_{—}}gC1qR · Lanadelumab_KAL_HMWK_gC1gR |
| 49 | dpreKAL_HMWK_gC1qR/dt = (kon_{HMWK}_{_{—}}_{gC1qR} · preKAL_HMWK · gC1qR − |
| | koff_{HMWK}_{_{—}}_{gC1qR} · preKAL_HMWK_gC1qR) − kcat_{preKAL}_{_{—}}_{Activation} · FXIIa_gC1qR · preKAL_HMWK_gC1qR · |
| | Num_to_Conc_converter/(km_{preKAL}_{_{—}}_{Activation} + |
| | preKAL_HMWK_gC1qR · Num_to_Conc_converter) − kdeg_{gC1qR} · preKAL_HMWK_gC1qR |
| 50 | dpreKAL_HMWK_gC1qR_degraded/dt = kdeg_{gC1qR} · |
| | preKAL_HMWK_gC1qR |
| 51 | Vₚᵣₒₓᵢₘₐₗ · dC1Inh/dt = (K12 · C1Inh_in_plasma · V_{medium} − k21 · C1Inh · |
| | Vₚᵣₒₓᵢₘₐₗ) − (kon_{C1Inh}_{_{—}}_{FXIIa} · C1InhFXIIa_gC1qR − koff_{C1Inh}_{_{—}}_{FXIIa} · |
| | C1Inh_FXIIa_gC1qR + k_{onC1Inh}_{_{—}}_{KAL} · C1InhKAL_HMWK_gC1qR − |
| | koff_{C1Inh}_{_{—}}_{KAL} · C1Inh_KAL_HMWK_gC1qR) · Num_to_Conc_converter · Vₚᵣₒₓᵢₘₐₗ |
| 52 | Vₚᵣₒₓᵢₘₐₗ · dC1Inh_FXIIa/dt = (k12 · C1Inh_FXIIa_in_plasma · V_{medium} − k21 · |
| | C1Inh_FXIIa · Vₚᵣₒₓᵢₘₐₗ) − |
| | (kon_{FXIIa}_{_{—}}_{gC1qR} · C1Inh_FXIIa · gC1qR − koff_{FXIIa}_{_{—}}_{gC1qR} · |
| | C1Inh_FXIIa_gC1qR) · Num_to_Conc_converter · Vₚᵣₒₓᵢₘₐₗ |
| 53 | Vₚᵣₒₓᵢₘₐₗ · dC1Inh_KAL_HMWK/dt = (k12 · C1Inh_KAL_HMWK_in_plasma · |
| | V_{medium} − k21 · C1Inh_KAL_HMWK · Vₚᵣₒₓᵢₘₐₗ) − (kon_{HMWK}_{_{—}}_{gC1qR} · |
| | C1Inh_KAL_HMWK · gC1qR − koff_{HMWK}_{_{—}}_{gC1qR} · |
| | C1Inh_KAL_HMWK_gC1qR) · Num_to_Conc_converter · Vₚᵣₒₓᵢₘₐₗ |
| 54 | Vₚᵣₒₓᵢₘₐₗ · dFXII/dt = (k12 · FXII_in_plasma · V_{medium} − k21 · FXII · Vₚᵣₒₓᵢₘₐₗ) − |
| | (kon_{FXII}_{_{—}}_{gC1qR} · FXII · gC1qR − koff_{FXII}_{_{—}}_{gC1qR} · FXII_gC1qR) · |
| | Num_to_Conc_converter · Vₚᵣₒₓᵢₘₐₗ |
| 55 | Vₚᵣₒₓᵢₘₐₗ · dFXIIa/dt = (k12 · FXIIa_in_plasma · V_{medium} − k21 · FXIIa · |
| | Vₚᵣₒₓᵢₘₐₗ) − (kon_{FXIIa}_{_{—}}_{gC1qR} · FXIIa · gC1qR − koff_{FXIIa}_{_{—}}_{gC1qR} · FXIIa_gC1qR) · |
| | Num_to_Conc_converter · Vₚᵣₒₓᵢₘₐₗ |
| 56 | Vₚᵣₒₓᵢₘₐₗ · dKAL_HK2Chain/dt = (k12 · XAL_HK2Chain_in_plasma · V_{medium} − |
| | k21 · KAL_HK2Chain · Vₚᵣₒₓᵢₘₐₗ) − (kon_{HK2Chain}_{_{—}}_{gC1qR} · KAL_HK2Chain · gC1qR − |
| | koff_{Hk2Chain}_{_{—}}_{gC1qR} · KAL_HK2Chain_gC1qR) · Num_to_Conc_converter · Vₚᵣₒₓᵢₘₐₗ |
| 57 | Vₚᵣₒₓᵢₘₐₗ · dKAL_HMWK/dt = (k12 · KAL_HMWK_in_plasma · V_{medium} − k21 · |
| | KAL_HMWK · Vₚᵣₒₓᵢₘₐₗ) − |
| | (kon_{HMWK}_{_{—}}_{gC1qR} · KAL_HMWK · gC1qR − koff_{HMWK}_{_{—}}_{gC1qR} · |
| | KAL_HMWK_gC1qR) · Num_to_Con_converter · Vₚᵣₒₓᵢₘₐₗ |
| 58 | Vₚᵣₒₓᵢₘₐₗ · dLanadelumab_KAL_HMWK/dt = |
| | (k12 · Lanadelumab_KAL_HMWK_in_plasma · V_{medium} − k21 |
| | Lanadelumab_KAL_HMWX · Vₚᵣₒₓᵢₘₐₗ) − (kon_{HMWK}_{_{—}}_{gC1qR} · |
| | Lanadelumab_KAL_HMWKgC1qR − koff_{HMWK}_{_{—}}_{gC1qR} · |
| | Lanadelumab_KAL_HMWK_gC1qR) · Num_to_Conc_converter · Vₚᵣₒₓᵢₘₐₗ |
| 59 | Vₚᵣₒₓᵢₘₐₗ · dpreKAL_HMWK/dt = (k12 · preKAL_HMWK_in_plasma · V_{medium} − |
| | k21 · preKAL_HMWK · Vₚᵣₒₓᵢₘₐₗ) − |
| | (kon_{HMWK}_{_{—}}_{gC1qR} · preKAL_HMWK · gC1qR − koff_{HMWK}_{_{—}}_{gC1qR} · preKAL_HMWK_gC1qR) · |
| | Num_to_Conc_converter · Vₚᵣₒₓᵢₘₐₗ |

One of the proteins implicated in the contact activation system PD model is Factor XII (FXII). FXII is a 80 kDa glycosylated protein consisting of a single polypeptide chain and circulates in plasma as a zymogen at a median concentration of 30 μg/ml (375 nM) in healthy individuals. Upon contact with anionic surfaces, in the presence of Zn²⁺ ions, FXII undergoes a conformational rearrangement leading to autoactivation or cleavage by kallikrein to generate FXIIa (the activated form of FXII).

Another protein implicated in the contact activation system PD model is prekallikrein (preKAL), a glycoprotein of molecular weight 85 kDa consisting of a single polypetide chain that circulates in plasma as a zymogen at a median concentration of 31 μg/ml (365 nM) in health individuals, with an estimated 75% bound to HMWK. preKAL binds to endothelial cells, platelets, and granulocytes in a Zn²⁺—dependent interaction via the preKAL-HMWK complex. The preKAL is cleaved by FXIIa resulting in KAL, the two-chain enzyme kallikrein. Prolylcarboxypeptidase (PRCP) has been identified as an endothelial cell activator of prekallikrein to kallikrein.

A third protein implicated in the contact activation system PD model is high molecular weight kinogen (HMWK), a 120 kDa non-enzymatic glycoprotein with a plasma concentration of 80 μg/ml (670 nM) in healthy individuals. The HMWK circulates in plasma both in free or complexed form (with preKAL or KAL). The binding affinities of HMWK to preKAL and KAL are similar, having a Kd of 12 nM and 15 nM respectively.

The contact activation system PD model shown in FIG. 4 models the binding, cleaving, and activation steps associated with the above contact factors as a cascade of molecular reactions.

The assembly of the kinin-kallikrein contact factor proteins on cell surfaces is mediated via uPAR (urokinase plasminogen activating receptor), and cofactors gC1q-R (complement protein C1q) and CK1 (cytokeratin 1). On the surface of endothelial cells, gC1q-R (with elevated levels of Zn⁺² ions, released from endothelial cells and activated platelets) is primarily responsible for assembly and activation of FXII/HMWK/preKAL. The model incorporates a number of assumptions based on known numbers of receptors, cofactors, and their complexes on endothelial cells. For example, gC1q-R is the most abundant with over 1 million per cell while uPAR (250,000/cell) and CK1 (72,000/cell) are less expressed. As gC1q-R/CK1 complex preferentially binds HMWK, and FXII binds primarily to uPAR within the CK1-uPAR complex, the model assumes that the least expressed CK1 is the limiting number to form the receptor complex in the activation of surface contact system. The model represents the cell surface with binding sites that may be characterized by the apparent site number and affinity to the different contact factors. The Zn⁺² dependency on binding affinity was not explicitly modeled and assumed that the effect is implicitly reflected in the reactions parameters where these factors play a role.

As described herein, excessive BK (bradykinin) causes an increase in blood vessel permeability, which allows fluid to pass through the blood vessel walls, causing subcutaneous or submucosal swelling. The cleavage of HMWK by kallikrein produces a two-chain cleaved HMWK (cHMWK) and the BK peptide. BK has a short half-life (less than 30 seconds in blood of most species) and strong affinity for the cell surface (0.5 nM). These properties of BK make it challenging to obtain reliable measurements of BK level. The contact activation system PD model may model and output levels of BK as well as cHMWK to provide a better understanding of HAE and the frequency, severity, and duration of acute attacks.

The contact activation system PD model may further incorporate known plasma concentrations of BK and cHMWK for healthy individuals and untreated HAE patients in both remission and while experiencing acute attack. For example, the contact activation system PD model may incorporate measured cHMWK for HAE patients with and without lanadelumab treatment. Based on the incorporated data, the contact activation system PD model may, in some embodiments, represent the formation and degradation of BK and cHMWK as molecular reactions. In some embodiments, the contact activation system PD model may represent the BK binding to BDKR-B2, and the degradation of the bound complex as molecular reactions.

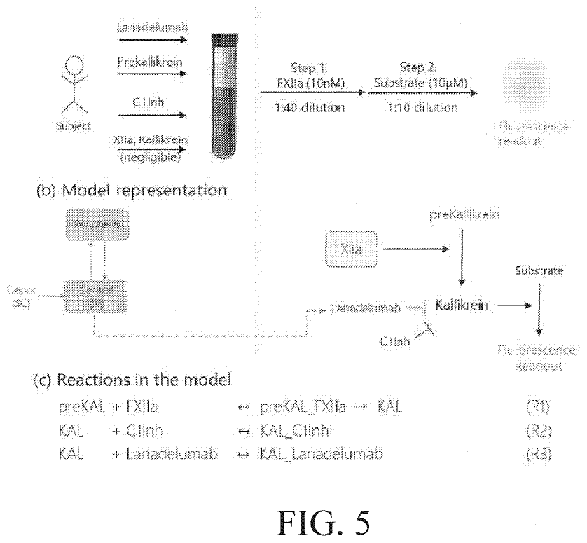

FIG. 5 illustrates an example in vitro assay procedure used in forming a fluorogenic assay PD model, in accordance with some embodiments of the technology described herein. The fluorogenic assay PD model may, in some embodiments, model the inhibition of kallikrein by a therapeutic intervention (e.g., administration of a drug such as lanadelumab), which may be measured and confirmed ex-vivo. In some embodiments, the fluorogenic assay PD model may be modeled first to parameterize and verify the inhibitory effect of a drug.

As described herein, Kallikrein (KAL) is a serine protease that plays a central role in activation of inflammation as well as in regulation of blood pressure and coagulation. In plasma, the activation of kallikrein is regulated by the physiological inhibitor, C1-INH. As described herein, HAE patients are deficient in functional C1-INH leading to irregularities in the kinin-kallikrein cascade which may, in turn, lead to an acute attack. Some treatment methods, including lanadelumab, for example, aim to inhibit excess formation of kallikrein by preventing cleavage of prekallikrein. The inventors have recognized that measuring the formation and inhibition of kallikrein ex-vivo using a fluorogenic assay, as described herein, provides a valuable way to isolate a subset of the kinin-kallikrein cascade, and to parameterize and verify the parameters within this subset.

FIG. 5 illustrates an in vitro assay procedure for measuring the inhibition of proteolytic activity of kallikrein by a therapeutic intervention. In the illustrated embodiment, the inhibition of kallikrein due to administration of lanadelumab is measured. The assay uses a peptide substrate for producing detectible fluorescence upon proteolysis catalyzed by kallikrein. The fluorogenic assay PD model, shown in FIG. 5(b) is represented by enzymatic reactions that form kallikrein from its precursor, prekallikrein, and that inhibit its function by the physiological inhibitor, C1-INH, and the administered drug, which in the illustrated embodiment, is lanadelumab. The reactions included in the PD model to represent the kallikrein formation and/or inhibition are shown in FIG. 5(c).

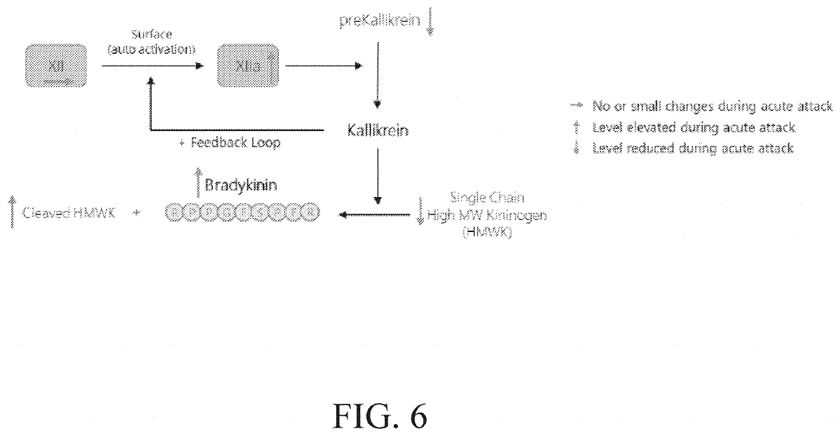

FIG. 6 illustrates an example illustration of protein level changes in HAE patients during an acute attack, in accordance with some embodiments of the technology described herein. In particular, FIG. 6 illustrates an example representation of the acute attack model. The acute attack model shown in FIG. 6 provides a representation of the changes in measured protein levels of the contact activation system. Changes in measured protein levels may provide an indicator of the existence of an acute attack and its severity. Studying the changes in measured protein levels over time may provide an indicator of acute attack duration and frequency. The effect of a therapeutic intervention on these indicators may be determined using the acute attack model in conjunction with one or more other models described herein.

As shown in FIG. 6, the acute attack model may indicate measured protein levels of the contact system (for example, in response to a stimulus, including, for example, a therapeutic intervention or an acute attack trigger causing autoactivation of Factor XII). For example, the acute attack model may indicate a measured level of any of FXII, FXIIa, preKAL, KAL, C1Inh, HMWK, cHMWK and/or % cHMWK, and/or BK. The inventors have recognized that certain proteins measurable by the QSP model described herein may be impractical or impossible to measure clinically (for example, levels of BK due to its relatively short half-life), and thus use of the QSP model may be advantageous in studying the effects of HAE and developing treatments for HAE.

The arrows illustrated in FIG. 6 indicate changes in protein levels during an acute attack as predicted by the QSP model. As described herein, an acute attack may arise in an individual having HAE when Factor XII is autoactivated, for example, due to one or more triggers, as described herein, into its activated form FXIIa. Thus, as shown in FIG. 6, there is an increase in levels of FXIIa. The activation of FXII cleaves prekallikrein to plasma kallikrein decreasing levels of prekallikrein and increasing levels of kallikrein. Cleavage of prekallikrein into plasma kallikrein in turn cleaves single-chain High Molecular Weight Kininogen (HMWK) into cleaved High Molecular Weight Kininogen (cHMWK). Thus, the levels of single-chain HMWK are decreased and levels of cHMWK are increased. Cleavage of HMWK liberates bradykinin, increasing BK levels and allowing bradykinin to bind to its receptors (BDKR-B2) on the surface of endothelial cells, causing an acute attack. Comparing the protein levels and relative change in protein levels to known amounts may allow the QSP model to predict characteristics of an acute HAE attack.

Virtual Population Development

As described herein, the kinin-kallikrein cascade leading to an acute attack in individuals with HAE may begin with autoactivation of FXII into its activated form FXIIa. Such autoactivation may happen at any time without warning. Autoactivation triggers may include stress, physical trauma, a surgical or a dental procedure, infection, hormonal changes, and mechanical pressure, for example. In some embodiments, the QSP model is configured on the assumption that each of these triggers may lead to a systematic perturbation in the contact system that autoactivates the kinin-kallikrein cascade leading to an HAE attack.

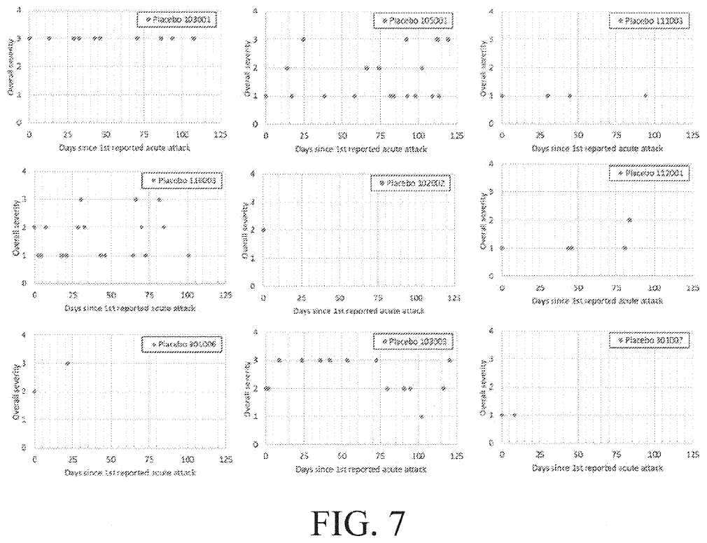

The severity and frequency of HAE attacks may vary widely from patient to patient and may also change over time, as shown in FIG. 7. FIG. 7 illustrates example clinical samples of time intervals between acute attacks in HAE patients, in accordance with some embodiments of the technology described herein. Given the variability in the frequency and severity of the acute attack as well as other patient-to-patient variabilities (for example, PK parameters), the inventors have recognized that modeling acute attacks over a population of patients (as opposed to using a prototypical patient in each state of the disease) may provide for a more accurate modeling and ability to better understand HAE and its potential treatments. Thus, in some embodiments, a virtual population of a plurality of HAE patients is used in conjunction with the QSP model.

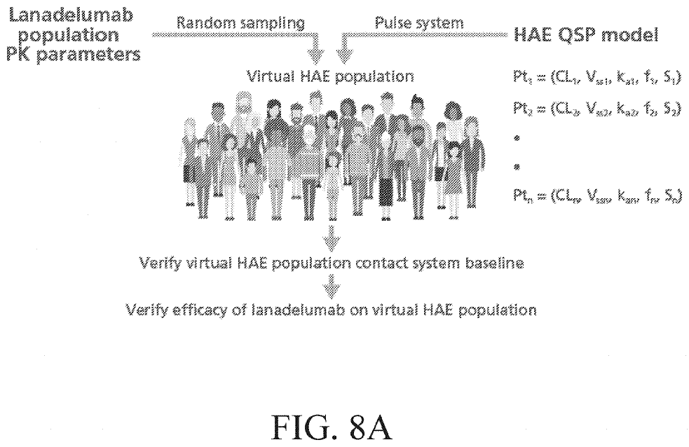

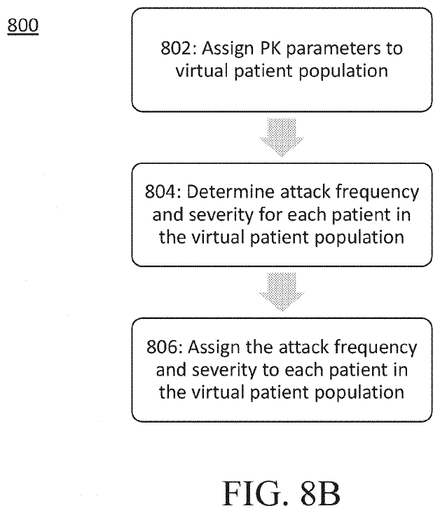

The virtual population may comprise a virtual data set comprising a plurality of data sets. Each data set (e.g., Patient₁) may represent an individual virtual patient of the virtual population and may have one or more variables defining one or more characteristics of the virtual patient. FIG. 8A illustrates an example representation of HAE virtual patient population capturing patient variability in pharmacokinetic parameters and propensity for acute attack represented by frequency (f) and severity (S), in accordance with some embodiments of the technology described herein. The virtual population may be input into the PD model to model HAE over a population of patients with HAE.

As shown in FIG. 8A, in some embodiments, each patient in the virtual population may be assigned PK parameters representing variability in the drug disposition for a particular patient (e.g., parameters indicating how a therapeutic intervention is impacted by biographical characteristics of the patient). In some embodiments, PK parameters are randomly assigned to virtual population, and may, in some embodiments, be based on clinical data. Example PK parameters may include body weight, age, sex, height, race, HAE type (Type I or Type II) and/or HAE attack severity.

In some embodiments, each of the virtual patients in the virtual population may be assigned disease predictive descriptors. Example disease predictive descriptors may include a virtual patient's propensity to experience an acute attack in the absence of therapeutic intervention, for example, baseline attack frequency, baseline attack severity, and/or baseline attack duration, as shown in FIG. 8. In some embodiments, the disease predictive descriptors, for example, attack frequency, are determined at least in part by simulation from a Poisson distribution informed by known data regarding the disease predictive descriptors. For example, although an HAE attack can happen at any time, individually, independent of the time since the last attack, collectively, over a time interval and a population, attacks tend to occur at a constant rate. Therefore, attack frequency may be modeled based on a Poisson process, in some embodiments, where the model generates an attack event based on an input of average attack frequency from a patient group of interest.

In some embodiments, a constant disease predictive descriptor may be applied to each patient in a virtual patient population. For example, in some embodiments, baseline attack duration may be equal for all patients of the virtual population (e.g., being set to 24 hours, in some embodiments)

For clinical studies, attack severity may be based on a score indicating the level of pain the patient is experiencing. The QSP model may be configured on the assumption that pain score is related to the level of BK caused by FXII autoactivation. Thus, attack severity may be represented as an increase in the FXII autoactivation in the QSP model, according to some embodiments.

FIG. 8B illustrates a method 800 for developing a virtual patient population comprising a plurality of virtual patients to simulate HAE, in accordance with some embodiments of the technology described herein. At act 802, PK parameters may be assigned to the virtual data set comprising a plurality of data sets representing a virtual population. For example, one or more PK parameters representing the disposition of a drug in a patient may be assigned to each patient in the virtual data set.

At act 804, one or more disease predictive descriptors may be determined for each patient in the virtual data set. For example, in some embodiments, an attack frequency and attack severity may be assigned for each patient in the virtual data set. At act 806, the disease predictive descriptors (e.g., the attack frequency and attack severity, in some embodiments) may be assigned to each patient in the virtual data set. In some embodiments, the virtual data set representing the virtual population may thereafter be input into the QSP model for modeling HAE among the patients of the virtual population.

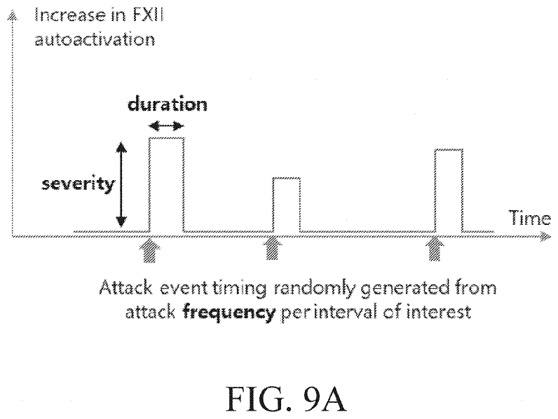

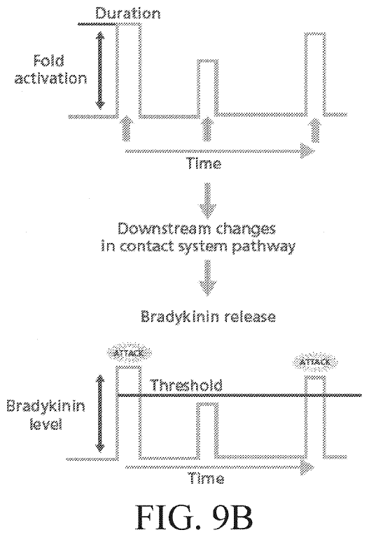

FIGS. 9A-9B illustrate example models of a trigger for an acute attack leading to auto-activation of the kinin-kallikrein pathway and production of elevated levels of bradykinin, in accordance with some embodiments of the technology described herein. In particular, FIGS. 9A-9B illustrate a relationship between FXII autoactivation and bradykinin levels. As described herein, the QSP model may model acute attacks by an autoactivation of FXII into its activated form, FXIIa. The cascade set off by the autoactivation may lead to downstream changes in the contact activation system, including an increase in Bradykinin levels which may bind to receptors on endothelial cells leading to swelling, as shown in FIG. 9B. The QSP model may determine an acute attack has occurred where BK levels have increased above a threshold level. In some embodiments, the threshold BK level signaling the existence of an acute attack may be based on literature and/or clinical data.

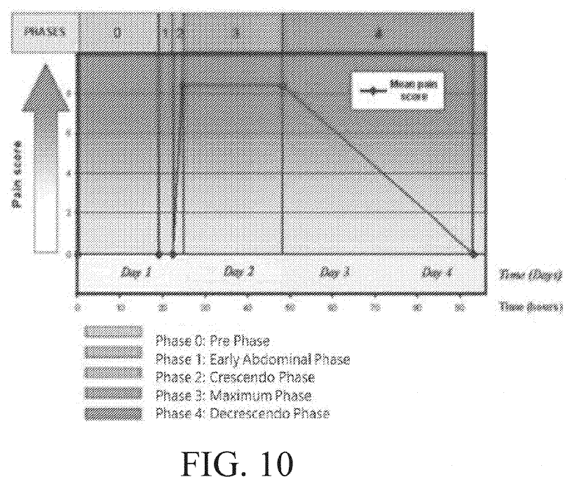

The duration of the attack may be represented by the period of time in which FXII autoactivation remains elevated and BK levels remain above the set threshold. FIG. 10 illustrates an example representation of different phases of an acute attack as indicated by a reported pain score in untreated HAE patients, in accordance with some embodiments of the technology described herein. As shown in FIG. 10, the pain stemming from swelling may increase during the first 8 to 24 hour period and then gradually subside over the next 24 to 72 hours. The reported clinical scores illustrated in FIG. 10 may inform the parameterization of acute attack duration for the virtual population.

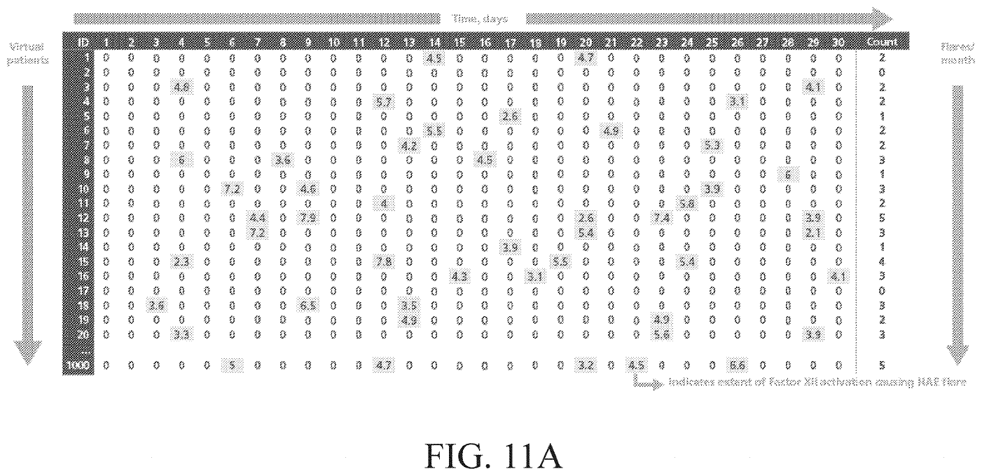

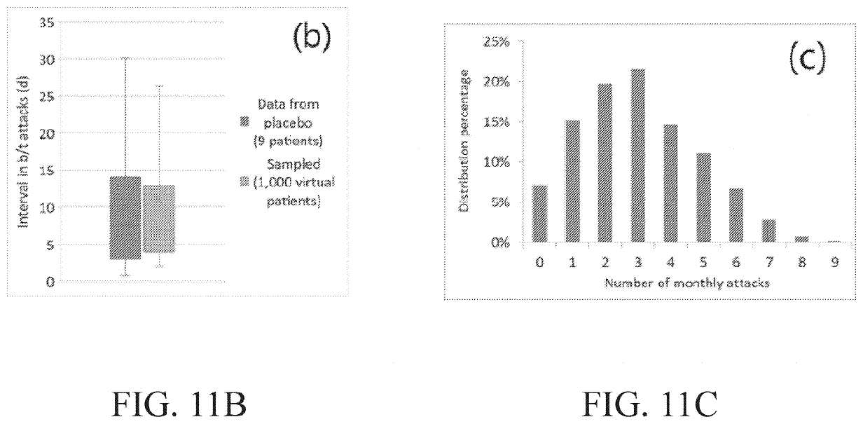

FIGS. 11A-11C illustrate examples of acute attack modeling in a virtual population, in accordance with some embodiments of the technology described herein. In particular, the results of virtual patient population development with assignment of PK parameters and disease predictive descriptors are shown in FIGS. 11A-11C. FIG. 11A illustrates the extent of FXII autoactivation causing an HAE flare over one month for a sampling of 20 patients of a virtual patient population of 1000 patients. FIG. 11C illustrates distribution of the number of monthly attacks per patient in the virtual population. FIG. 11B illustrates simulated attack frequency distribution for the virtual population compared to clinical data from a group of patients having HAE. FIG. 11B illustrates that the attack frequency data for the virtual population shows good agreement with clinical data.

As described herein, the virtual population may be input into the PD model. The contact activation system PD model may predict the level of BK to determine whether an acute attack has occurred in response to a trigger. For example, when a trigger even occurs, the state of the acute attack may be predicted by determining whether the BK level output by the contact activation system PD model exceeds a known threshold. In this way, the QSP model may provide for analysis of the contact system including during an HAE attack and evaluation of the effectiveness of new and existing treatment modalities for HAE.

Quantitative Systems Pharmacology Model Parameterization

The QSP model may be parameterized with existing clinical and literature data to provide for more accurate modeling of HAE. For example, the fluorogenic assay PD model may be parameterized with enzyme reaction rates known from literature. The contact activation system PD model may be parameterized with clinical data of protein levels of healthy subjects and subjects with HAE. The acute attack clinical outcome model may be parameterized with clinical data of protein levels of HAE patients under acute attack and time intervals of acute attacks in untreated patients with HAE. The PK model may be parameterized with clinical data. Table 4 gives a list of model parameters for the QSP model. Table 5 gives a list of model assumptions implemented in the model.

TABLE 4
List of model parameters (SS denotes steady state)

| | | | |
|---|---|---|---|
| Parameter | Description | Unit | Value |

In Vascular Space

| | | | |
|---|---|---|---|
| Kd_KAL_HK2Chain | KD for “KAL_in_plasma + | nM | 72 |
| | HK2Chain_in_plasma ↔ | | |
| | KAL_HK2Chain_in_plasma” | | |
| Kd_KAL_HMWK | KD for “KAL_in_plasma + | nM | 15 |
| | HMWK_in_plasma ↔ | | |
| | KAL_HMWK_in_plasma” | | |
| Kd_preKAL_HMWK | KD for “preKAL_in_plasma + | nM | 12 |
| | HMWK_in_plasma ↔ | | |
| | preKAL_HMWK_in_plasma” | | |
| kdeg_BK | Degradation rate for BK | 1/h | 55.452 |
| kdeg_Bound_C1Inh | Degradation rate for bound C1Inh | 1/h | 13.863 |
| kdeg_C1Inh | Degradation rate for C1Inh | 1/h | 0.0165 |
| kdeg_cHMWK | Degradation rate for HK2Chain | 1/h | 0.0619 |
| kdeg_FXII | Degradation rate for FXII | 1/h | 0.0116 |
| kdeg_FXIIa | Degradation rate for FXIIa | 1/h | 8.138 |
| kdeg_HMWK | Degradation rate for HMWK | 1/h | 0.0044 |
| kdeg_KAL | Degradation rate for KAL | 1/h | 8.138 |
| kdeg_preKAL | Degradation rate for preKAL | 1/h | 0.0289 |
| koff_KAL_HK2Chain | off rate for KAL & HK2Chain binding | 1/h | 318.816 |
| | event | | |
| koff_KAL_HMWK | off rate for KAL & HMWK binding event | 1/h | 66.42 |
| koff_preKAL_HMWK | off rate for preKAL & HMWK binding | 1/h | 53.136 |
| | event | | |
| kon_KAL_HK2Chain | kon for “KAL_in_plasma + | 1/(M*h) | 4.428 |
| | HK2Chain_in_plasma ↔ | | |
| | KAL_HK2Chain_in_plasma” | | |
| kon_KAL_HMWK | kon for “KAL_in_plasma + | 1/(M*h) | 4.428 |
| | HMWK_in_plasma ↔ | | |
| | KAL_HMWK_in_plasma” | | |
| kon_preKAL_HMWK | kon for “preKAL_in_plasma + | 1/(M*h) | 4.428 |
| | HMWK_in_plasma ↔ | | |
| | preKAL_HMWK_in_plasma” | | |
| ksyn_C1Inh | Synthesis rate for C1Inh | nM/h | 11.883 HAE/ |
| | | | 39.608 Healthy |
| ksyn_FXII | Synthesis rate for FXII | nM/h | 10.83 |
| ksyn_HMWK | Synthesis rate for HMWK | nM/h | 39.933 |
| ksyn_preKAL | Synthesis rate for preKAL | nM/h | 41.589 |
| Vmedium | Per endothelial cell based plasma volume | L | 1.23E−12 |

In Proximal Space

| | | | |
|---|---|---|---|
| BDKRB2_per_Cell_SS | The number of BDKRB2 per cell at steady | — | 100,000 |
| | state | | |
| gC1qR_per_Cell_SS | The number of gC1qR per cell at steady | — | 100,000 |
| | state | | |
| kcat_FXII_Activation | kcat for FXII activation (S: FXII_gC1qR; | 1/h | 15 |
| | E: KAL_HMWK_gC1qR; P: | | |
| | FXIIa_gC1qR) | | |
| kcat_FXII_AutoActivation | kcat for FXII auto-activation (S: | 1/h | 0.0475 |
| | FXII_gC1qR; E: FXII_gClqR; P: | | |
| | FXIIa_gC1qR) | | |
| kcat_HMWK_cleavage | kcat for cleavage of HMWK | 1/h | 394.7 |
| kcat_preKAL_Activation | kcat for preKAL activation (S: | 1/h | 18 |
| | preKAL_HMWK_gC1qR; E: | | |
| | FXIIa_gC1qR; P: KAL_HMWK_gC1qR) | | |
| Kd_BK_BDKRB2 | KD for “BK + BDKRB2 ↔ BK_BDKRB2” | nM | 0.5 |
| Kd_FXII_gC1qR | KD for “FXII + gC1qR ↔ FXII_gC1qR” | nM | 144 |
| Kd_FXIIa_gC1qR | KD for “FXIIa + gC1qR ↔ FXIIa_gC1qR” | nM | 144 |
| Kd_HK2Chain_gC1qR | KD for “KAL_HK2Chain + gC1qR ↔ | nM | 10.35 |
| | KAL_HK2Chain_gC1qR” | | |
| Kd_HMWK_gC1qR | KD for “preKAL_HMWK + gC1qR ↔ | nM | 10.35 |
| | preKAL_HMWK_gC1qR” | | |
| kdeg_BDKRB2 | Degradation rate for BDKRB2 receptors | 1/h | 0.3466 |
| kdeg_gC1qR | Degradation rate for receptor complex on | 1/h | 0.3466 |
| | endothelial cell surface | | |
| kdeg_Lanadelumab_KAL_HMWK_gC1qR | Degradation rate for Lanadelumab bound | nM | 0.3466 |
| | with KAL_HMWK_gC1qR receptor | | |
| | complex | | |
| Km_FXII_Activation | Km for FXII activation (S: FXII_gC1qR; | nM | 510 |
| | E: KAL_HMWK_gC1qR; P: | | |
| | FXIIa_gC1qR) | | |
| Km_FXII_AutoActivation | Km for FXII auto-activation (S: | nM | 110 |
| | FXII_gC1qR; E: FXII_gC1qR; P: | | |
| | FXIIa_gC1qR) | | |
| Km_preKAL_Activation | Km for preKAL activation (S: | nM | 91 |
| | preKAL_HMWK_gC1qR; E: | | |
| | FXIIa_gC1qR; P: KAL_HMWK_gC1qR) | | |
| koff_BK_BDKRB2 | off rate for BK & BDKRB2 receptor | 1/h | 18 |
| | binding event | | |
| koff_FXII_gC1qR | off rate for FXII & surface receptor | 1/h | 63.763 |
| | binding event | | |
| koff_FXIIa_gC1qR | off rate for FXIIa & surface receptor | 1/h | 63.763 |
| | binding event | | |
| koff_HK2Chain_gC1qR | off rate for HK2Chain & surface receptor | 1/h | 4.583 |
| | binding event | | |
| koff_HMWK_gC1qR | off rate for HMWK & surface receptor | 1/h | 4.583 |
| | binding event | | |
| kon_BK_BDKRB2 | kon for “BK + BDKRB2 ↔ | 1/(M*h) | 56 |
| | BK_BDKRB2” | | |
| kon_FXII_gC1qR | kon for “FXII + gC1qR ↔ FXII_gC1qR” | 1/(M*h) | 0.4428 |
| kon_FXIIa_gC1qR | kon for “FXIIa + gC1qR ↔ FXIIa_gC1qR” | 1/(M*h) | 0.4428 |
| kon_HK2Chain_gC1qR | kon for “KAL_HK2Chain + gC1qR ↔ | 1/(M*h) | 0.4428 |
| | KAL_HK2Chain_gC1qR” | | |
| kon_HMWK_gC1qR | kon for “preKAL_HMWK + gC1qR ↔ | 1/(M*h) | 0.4428 |
| | preKAL_HMWK_gC1qR” | | |
| ksyn_BDKRB2 | Synthesis rate for BDKRB2 receptors | number/cell/h | 34657.359 |
| ksyn_gC1qR | Synthesis rate for receptor complex on | number/cell/h | 34657.359 |
| | endothelial cell surface | | |
| Vₚᵣₒₓᵢₘₐₗ | Proximal space volume near cell surface | L | 8.00E−15 |
| | for each endothelial cell | | |

In Vascular and Proximal Space

| | | | |
|---|---|---|---|
| Kd_C1Inh_FXIIa | KD for “C1Inh_in_plasma + | nM | 1720 |
| | FXIIa_in_plasma ↔ | | |
| | C1Inh_FXIIa_in_plasma” and “C1Inh + | | |
| | FXIIa_gC1qR ↔ C1Inh_FXIIa_gC1qR” | | |
| Kd_C1Inh_KAL | KD for “C1Inh_in_plasma + | nM | 150 |
| | KAL_in_plasma ↔ | | |
| | C1Inh_KAL_in_plasma”, | | |
| | “C1Inh_in_plasma + | | |
| | KAL_HMWK_in_plasma ↔ | | |
| | C1Inh_KAL_HMWK_in_plasma”, and | | |
| | “C1Inh + KAL_HMWK_gC1qR ↔ | | |
| | C1Inh_KAL_HMWK_gC1qR” | | |
| Kd_KAL_Lanadelumab | KD for “KAL_in_plasma + | nM | 0.12 |
| | Lanadelumab_in_plasma ↔ | | |
| | Lanadelumab_KAL_in_plasma”, | | |
| | “KAL_HMWK_in_plasma + | | |
| | Lanadelumab_in_plasma ↔ | | |
| | Lanadelumab_KAL_HMWK_in_plasma”, | | |
| | and “KAL_HMWK_gC1qR + | | |
| | Lanadelumab ↔ | | |
| | Lanadelumab_KAL_HMWK_gC1qR” | | |
| koff_C1Inh_FXIIa | koff for “C1Inh_in_plasma + | 1/h | 229.104 |
| | FXIIa_in_plasma ↔ | | |
| | C1Inh_FXIIa_in_plasma” and “C1Inh + | | |
| | FXIIa_gC1qR ↔ C1Inh_FXIIa_gC1qR” | | |
| koff_C1Inh_KAL | koff for “C1Inh_in_plasma + | 1/h | 9.18 |
| | KAL_in_plasma ↔ | | |
| | C1Inh_KAL_in_plasma”, | | |
| | “C1Inh_in_plasma + | | |
| | KAL_HMWK_in_plasma ↔ | | |
| | C1Inh_KAL_HMWK_in_plasma”, and | | |
| | “C1Inh + KAL_HMWK_gC1qR ↔ | | |
| | C1Inh_KAL_HMWK_gC1qR” | | |
| koff_KAL_Lanadelumab | koff for “KAL_in_plasma + | 1/h | 1.452 |
| | Lanadelumab_in_plasma ↔ | | |
| | Lanadelumab_KAL_in_plasma”, | | |
| | “KAL_HMWK_in_plasma + | | |
| | Lanadelumab_in_plasma ↔ | | |
| | Lanadelumab_KAL_HMWK_in_plasma”, | | |
| | and “KAL_HMWK_gC1qR + | | |
| | Lanadelumab ↔ | | |
| | Lanadelumab_KAL_HMWK_gC1qR” | | |
| kon_C1Inh_FXIIa | kon for “C1Inh_in_plasma + | 1/(nM*h) | 0.1332 |
| | FXIIa_in_plasma ↔ | | |
| | C1Inh_FXIIa_in_plasma” and “C1Inh + | | |
| | FXIIa_gC1qR ↔ C1Inh_FXIIa_gC1qR” | | |
| kon_C1Inh_KAL | kon for “C1Inh_in_plasma + | 1/(nM*h) | 0.0612 |
| | KAL_in_plasma ↔ | | |
| | C1Inh_KAL_in_plasma”, | | |
| | “C1Inh_in_plasma + | | |
| | KAL_HMWK_in_plasma ↔ | | |
| | C1Inh_KAL_HMWK_in_plasma”, and | | |
| | “C1Inh + KAL_HMWK_gC1qR ↔ | | |
| | C1Inh_KAL_HMWK_gC1qR” | | |
| kon_KAL_Lanadelumab | kon for “KAL_in_plasma + | 1/(nM*h) | 12.096 |
| | Lanadelumab_in_plasma ↔ | | |
| | Lanadelumab_KAL_in_plasma”, | | |
| | “KAL_HMWK_in_plasma + | | |
| | Lanadelumab_in_plasma ↔ | | |
| | Lanadelumab_KAL_HMWK_in_plasma”, | | |
| | and “KAL_HMWK_gC1qR + | | |
| | Lanadelumab ↔ | | |
| | Lanadelumab_KAL_HMWK_gC1qR” | | |

Exchange Between Vascular and Proximal Space

| | | | |
|---|---|---|---|
| K12 | Species exchange rate from plasma to | 1/h | 0.2341 |
| | proximal space | | |
| K21 | Species exchange rate from proximal space | 1/h | 36 |
| | to plasma | | |
| K12_BK | BK exchange rate from plasma to proximal | 1/h | 7.0244 |
| | space | | |
| K21_BK | BK exchange rate from proximal space to | 1/h | 1080 |
| | plasma | | |

TABLE 5
List of model assumptions

| | |
|---|---|
| 1 | The least expressed CK1 cofactor is the limiting number to form the receptor complex of |
| | gCq1-R/CK1/uPAR in the activation of surface contact system. An apparent site number |
| | and affinity to different contact factors are used. |
| 2 | The effects of Zn⁺² dependency on binding affinities and the endothelial cell prekallikrein |
| | activator (PRCP) are not explicitly included in the model and their effects are assumed to |
| | be implicitly reflected in the model parameters. |
| 3 | The exchange rate between the vascular space and the proximal space is assumed to be of |
| | the same order as the vascular volume circulation time, approximately 100 seconds. |
| 4 | Various triggers of HAE attack (e.g., stress, physical trauma, a surgical or a dental |
| | procedure, infection, hormonal changes, mechanical pressure) are assumed to lead to a |
| | systematic perturbation that autoactivates the kinin-kallikrein pathway in the contact |
| | activation system. |
| 5 | Rise in the pain is triggered by an acute attack and the model represents the duration of the |
| | attack as the time period over which the level of FXII autoactivation remains elevated. |
| 6 | The same inhibitory activity of lanadelumab on KAL in plasma applies to that of KAL |
| | bound to the surface, and that the inhibitory activity of C1INH on KAL and FXIIa in |
| | plasma would be the same as on the surface. |

As described herein, the PK model may provide a dose level profile to the PD models. The parameters of the PK model illustrated in FIG. 2 include central volume (V_{c}), peripheral volume (Vₚ), flow rate between central and peripheral compartments (Q), central clearance rate (CL), absorption rate (ka), and bioavailability (F). Each of the parameters may be calibrated and/or fixed based on literature and clinical data.

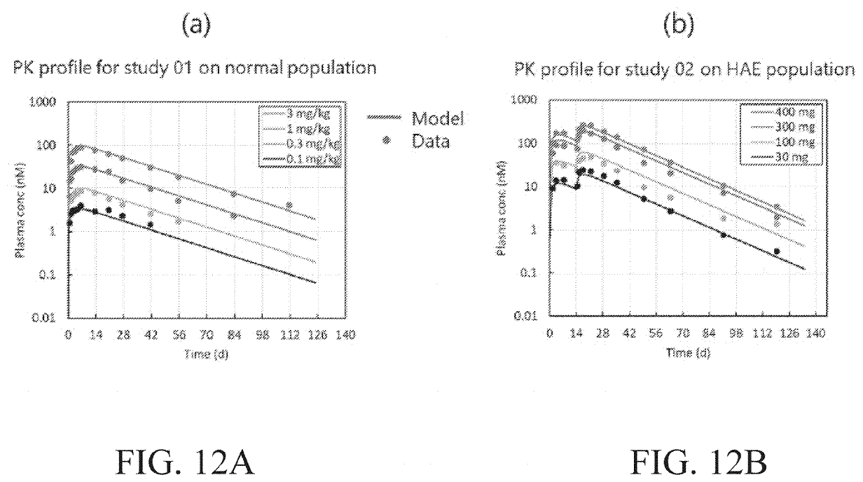

FIGS. 12A-12B illustrate examples of simulated PK profiles using the example PK model of FIG. 3, in accordance with some embodiments of the technology described herein. FIGS. 12A-12B illustrates PK profiles (illustrated by lines) simulated based on the PK model of FIG. 2 compared to clinical data (illustrated by symbols). FIG. 12A illustrates PK profiles for individuals without HAE, while FIG. 12B illustrates PK profiles for individuals with HAE.

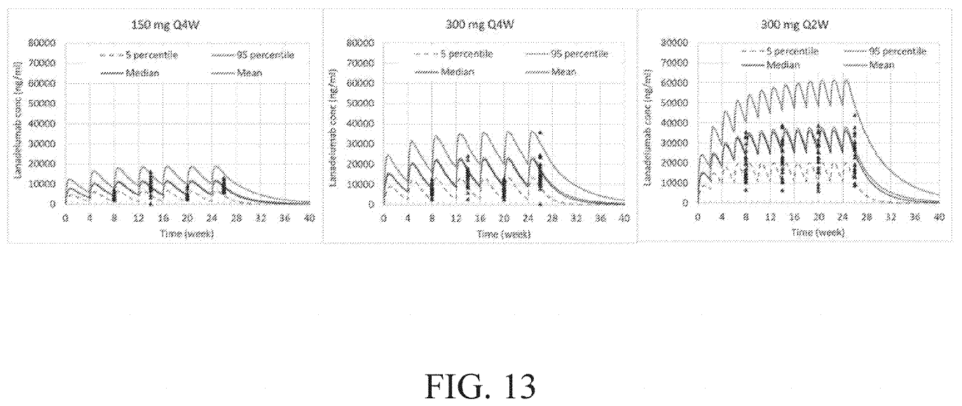

FIG. 13 illustrates examples of simulated PK profiles using an example one-compartment PK model, in accordance with some embodiments of the technology described herein. In particular, FIG. 13 illustrates PK profiles for HAE patients treated with lanadelumab according to different dosage regimens (150 mg Q4W, 300 mg Q4W, and 300 mg Q2W). FIG. 13 illustrates that the majority of the data is captured within the 5^{th} to 95^{th} percentile of the model prediction. The simulated PK profiles (illustrated by lines) are compared to clinical data (illustrated by symbols) in FIG. 13, showing good agreement with the clinical data.

The fluorogenic assay PD model may be parameterized with clinical data of measured levels of kallikrein activity inhibited by therapeutic intervention (e.g., administration of lanadelumab) measured by the in vitro assay procedure described with respect to FIG. 5. The fluorogenic assay PD model may receive prekallikrein level in plasma, kallikrein level in plasma, plasma C1 inhibitor level for normal population, and plasma C1 inhibitor level for HAE type I population as input and include binding affinity of the administered drug to kallikrein, binding affinity of CInh to kallikrein, Km for activation of prekallikrein by FXIIa, and kcat for activation of prekallikrein by FXIIa as parameters.

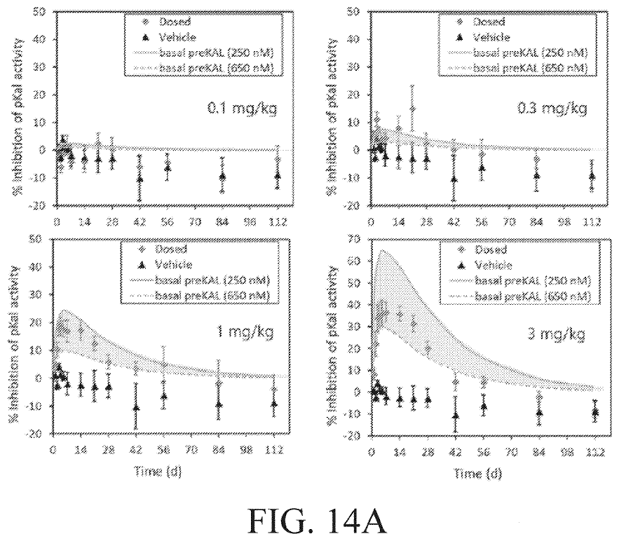

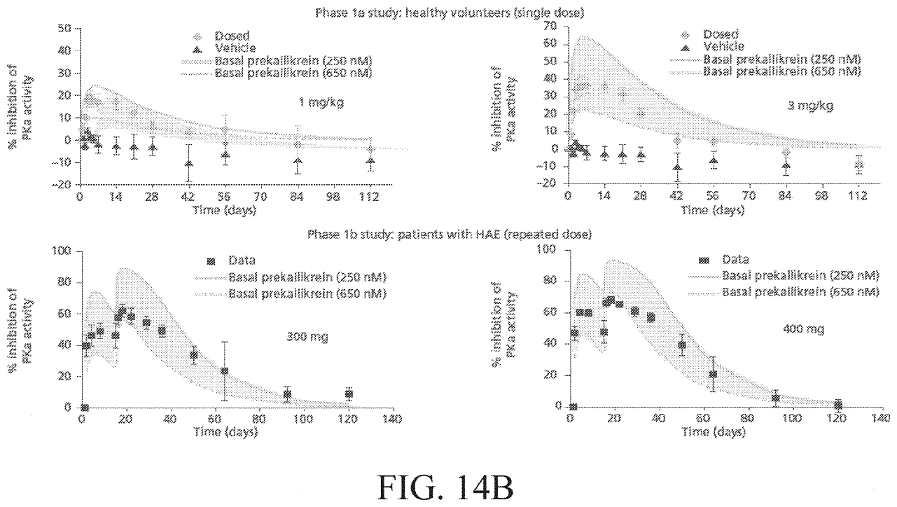

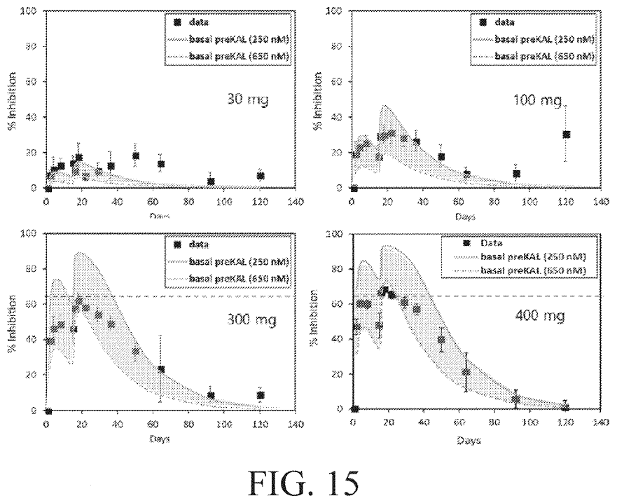

FIGS. 14A-15 illustrate examples of simulation output using the PD model of FIG. 5 representing the fluorescence assay compared with clinical data of measured level of kallikrein inhibition activity, in accordance with some embodiments of the technology described herein. Simulated results for plasma kallikrein inhibition are in good agreement with clinical data for healthy patients, untreated HAE patients, and treated HAE patients. The dotted line in FIG. 15 illustrates the % inhibition for FDA approved 30 mg dose of Ecallantide (Kalbitor).

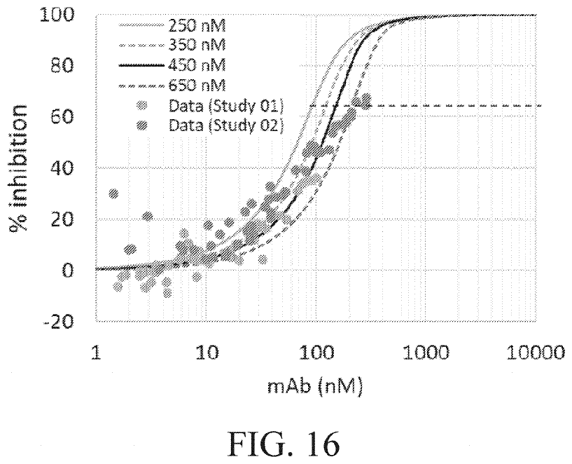

In some embodiments, the QSP model, and more particularly, the fluorogenic assay model may be used to estimate an effectiveness of a therapeutic intervention by determining whether the therapeutic intervention inhibits plasma kallikrein and to what extent. FIG. 16 illustrates dose-dependent inhibition of kallikrein by lanadelumab for a range of prekallikrein levers (250-650 nM) reported in the literature, in accordance with some embodiments of the technology described herein. In some embodiments, the QSP model may be used to determine the effectiveness of a particular dosage of a drug, for example, by determining whether and to what extent the dosage inhibits plasma kallikrein.

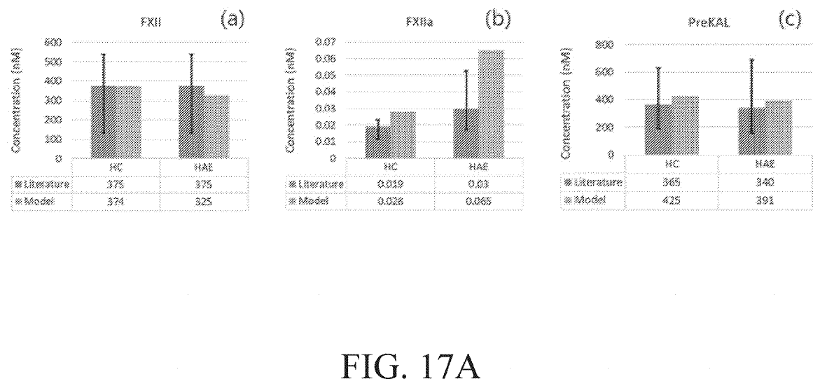

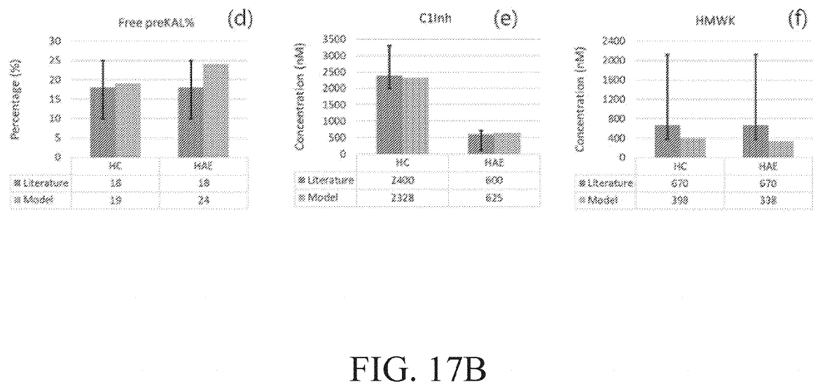

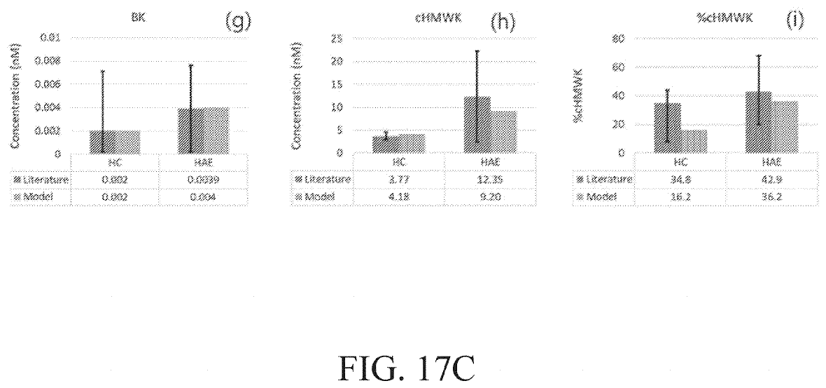

The reactions and governing equations which may be implemented in the contact activation system PD model are shown in Table 3. Components of the contact activation system PD model may be parameterized with literature data and/or calibrated by data from one or more other models of the QSP model. For example, such components may include, in some embodiments, FXII, FXIIa, prekallikrein, free prekallikrein percentage, C1-INH, HMWK, BK, cHMWK, and/or percentage of cHMWK. FIGS. 17A-17C illustrate comparisons of steady-state levels of proteins of the HAE contact system reported in literature and predicted levels using the contact activation system PD model of FIG. 2, in accordance with some embodiments of the technology described herein. FIGS. 17A-17C show that the contact activation system PD model output is in good agreement with protein level data at steady-state.

The acute attack model may be parameterized to calibrate the severity of an attack trigger so that the levels of proteins in the kinin-kallikrein cascade (e.g., cHMWK, BK, etc.) from simulated HAE patients under acute attack are in agreement with pre-does clinical data. As described herein, attack severity may be represented in the acute attack model by an increase in the autoactivation of FXII.

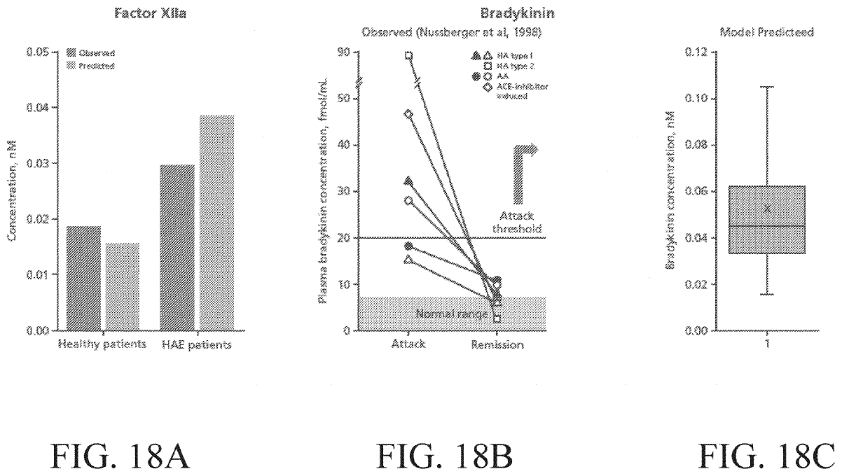

FIGS. 18A-18C illustrate example comparisons of bradykinin and factor XIIa levels in clinical data and predicted data using the PD model of FIG. 2, in accordance with some embodiments of the technology described herein. FIG. 18A illustrates a comparison between simulated data and clinical data for Factor XIIa levels in healthy patients and HAE patients. FIGS. 18B and 18C illustrate predicted levels of BK due to the increase in FXIIa.

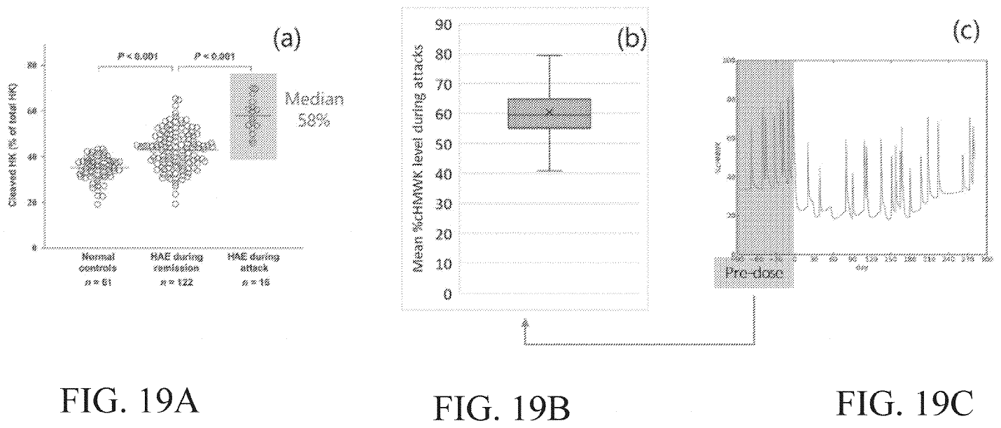

FIGS. 19A-19C illustrates examples comparisons of cHMWK levels in clinical data from HAE patients under acute attack and predicted data using the PD model of FIG. 2, in accordance with some embodiments of the technology described herein. FIG. 19A illustrates measured percentage cHMWK levels in patients without HAE, and patients with HAE during attack and during remission. FIG. 19B illustrates that the predicted data output by the acute attack model is consistent with the measured clinical data. FIG. 19C illustrates a temporal profile of cHMWK over time, including before and after therapeutic intervention. Percentage cHWMK represents the percentage of cHMWK relative to the total of cHMWK and HMWK.

### Computer Implementations of Example QSP Models

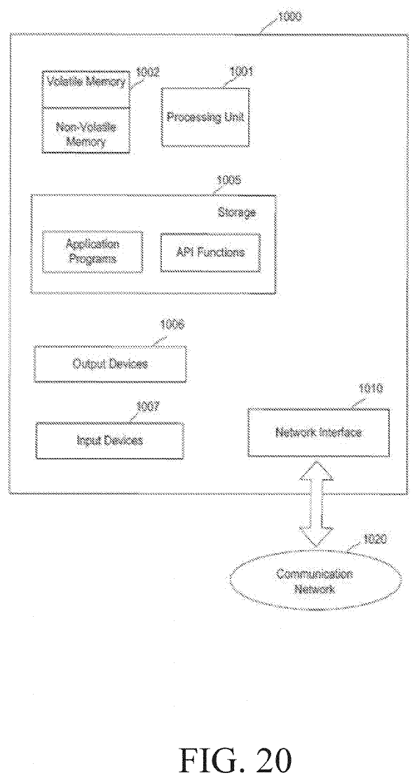

The QSP model and further aspects of the technology described herein may be implemented using a computer. FIG. 20 shows, schematically, an illustrative computer 1000 on which any aspect of the present disclosure may be implemented. In the embodiment shown in FIG. 20, the computer 1000 includes a processing unit 1001 having one or more computer hardware processors and one or more articles of manufacture that comprise non-transitory computer-readable storage media (e.g., system memory 1002) that may include, for example, volatile and/or non-volatile memory. The memory 1002 may store one or more instructions to program the processing unit 1001 to perform any of the functions described herein. The computer 1000 may also include other types of non-transitory computer-readable media, such as storage 1005 (e.g., one or more disk drives) in addition to the system memory 1002. The storage 1005 may also store one or more application programs and/or external components used by application programs (e.g., software libraries), which may be loaded into the memory 1002. To perform any of the functionality described herein, processing unit 1001 may execute one or more processor-executable instructions stored in the one or more non-transitory computer-readable storage media (e.g., memory 1002, storage 1005), which may serve as non-transitory computer-readable storage media storing processor-executable instructions for execution by the processing unit 1001.

The computer 1000 may have one or more input devices and/or output devices, such as devices 1006 and 1007 illustrated in FIG. 20. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards and pointing devices, such as mice, touch pads, and digitizing tablets. As another example, the input devices 1007 may include a microphone for capturing audio signals, and the output devices 1006 may include a display screen for visually rendering, and/or a speaker for audibly rendering, recognized text.

As shown in FIG. 20, the computer 1000 may also comprise one or more network interfaces (e.g., the network interface 10010) to enable communication via various networks (e.g., the network 10020). Examples of networks include a local area network or a wide area network, such as an enterprise network or the Internet. Such networks may be based on any suitable technology and may operate according to any suitable protocol and may include wireless networks, wired networks or fiber optic networks.

In some embodiments, the QSP model may be used in a computer-implemented method, as described herein. In some embodiments, at least one non-transitory computer-readable storage medium is provided having processor-executable instructions that, when executed by at least one computer-hardware processor, cause the computer-hardware to perform a computer-implemented method which utilizes the QSP model described herein.

Quantitative Systems Pharmacology Model Verification

The parameterized models may be verified in a simulation to determine that model results for treated patients with HAE match clinical data to ensure that the QSP model may accurately model HAE and provide evaluation of new existing treatment modalities. For example, the contact activation system PD model may be applied to verify the inhibitory effect of a therapeutic intervention (e.g., administration of lanadelumab) on HAE patients by comparing simulation results to biomarker data (e.g., cHMWK levels). The acute attack model may be applied to verify the inhibitory effect of a therapeutic intervention (e.g., administration of lanadelumab) on HAE patients by comparing simulation results to biomarker data (e.g., cHMWK levels). The acute attack model may further be applied to investigate the sensitivity of monthly attack rates to attack severity, attack frequency, and binding affinity of an administered drug (e.g., lanadelumab) as well as the sensitivity of the BK level to system parameters of the model.

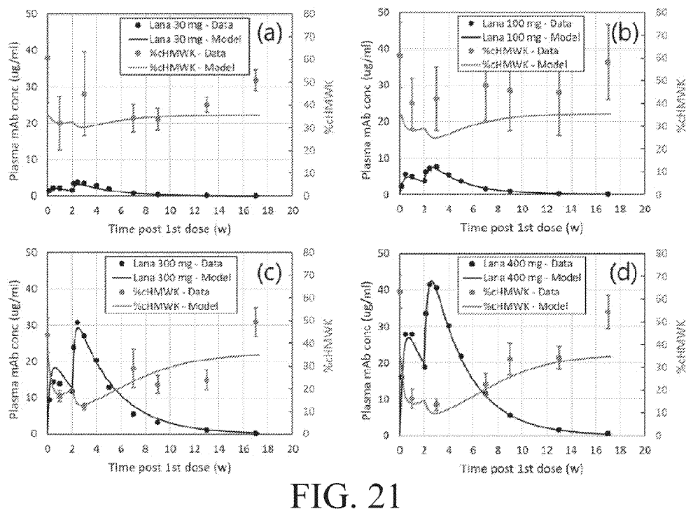

FIG. 21 illustrates comparisons of cHMWK levels from clinical data to simulation output from the Contact Surface Activation model of FIG. 2 in HAE patients treated with different dosages of lanadelumab, in accordance with some embodiments of the technology described herein. In particular, FIG. 21 illustrates graphs comparing the level of cHMWK from clinical data to the simulation output from the contact activation system PD model for HAE patients treated with 30 mg, 100 mg, 300 mg, and 400 mg of lanadelumab. Lanadelumab concentration in plasma obtained from the PK model is also shown. The simulation results correctly match clinical data and show an inverse correlation between the concentration of an administered drug and cHMWK. Thus, the simulation results confirm suppression of BK (e.g., lower percentage cHMWK for higher dosages) which increases with dosage.

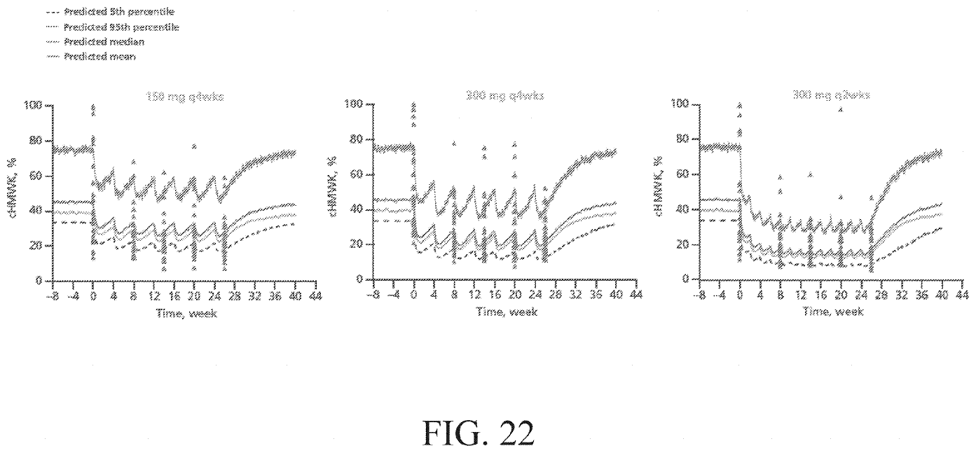

FIG. 22 illustrates comparisons of cHMWK levels from clinical data to simulation output from the Acute Attack Model of FIG. 2 in HAE patients treated with different dosages of lanadelumab, in accordance with some embodiments of the technology described herein. FIG. 22 compares percentage cHMWK output by the acute attack model with clinical data for HAE patients treated with different dose regiments (150 mg Q4W, 300 mg Q4W, 300 mg Q2W), and lanadelumab concentration output from the PK model. FIG. 22 illustrates that percentage cHMWK decreases with higher doses (150 mg Q4W vs. 300 mg Q4W) and more frequent doses (300 mg Q4W vs. 300 mg Q2W). The simulation results confirm this trend.

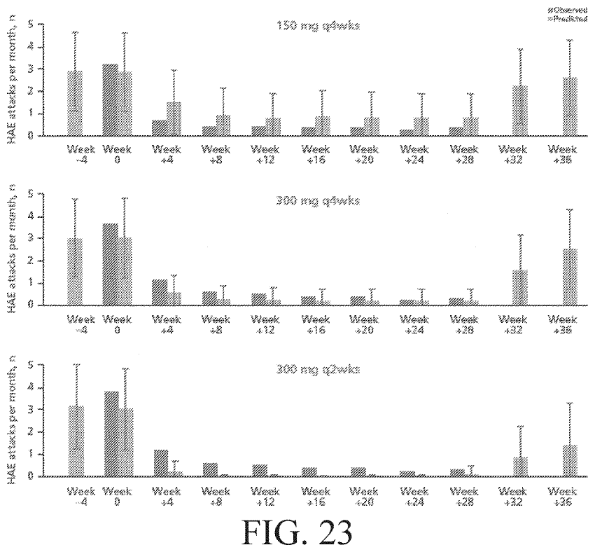

FIG. 23 illustrates comparisons of HAE acute attack rates from clinical data to simulation output from the Acute Attack Model for HAE patients treated with different dosages of lanadelumab, using the same data source and simulation as shown in FIG. 22. FIG. 23 compares the number of HAE acute attacks averaged over a month. Both the clinical data and simulation output illustrate a reduction in the number of HAE acute attacks for all dose regimens, confirming that all dose regimens (150 mg Q4W, 300 mg Q4W, and 300 mg Q2W lanadelumab) are effective in suppressing HAE acute attack frequency. The simulation results in

The simulation output reflected in FIGS. 21-23 clinical study was obtained using the QSP model with a virtual population of 1000 virtual patients. However, the virtual population may have any suitable number of virtual patients (e.g., at least 100 virtual patients, at least 500 virtual patients, at least 1000 virtual patients). The BK threshold for determining the occurrence of an acute attack was 20 pM of BK, though other thresholds are possible (e.g., any threshold between and inclusive of 15 pM to 90 pM, for example).

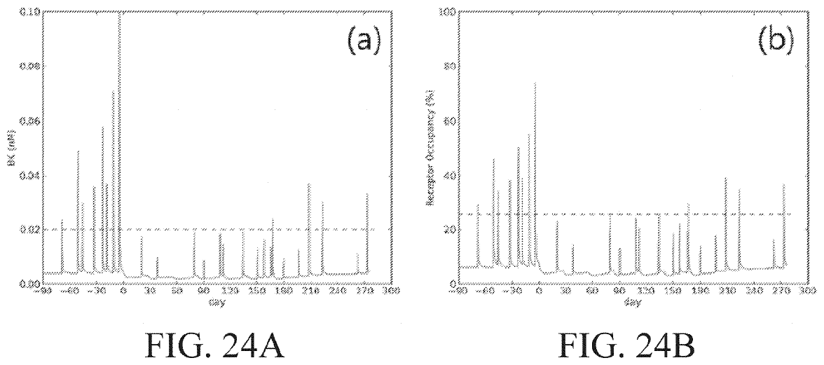

In some embodiments, the threshold for determining the occurrence of an acute attack may be based on the receptor occupancy (RO) of the BDKR-B2 receptor to which BK binds. FIGS. 24A-24B illustrate example time profiles of bradykinin levels and BDKR-B2 receptor occupancy for virtual patients being treated with lanadelumab, in accordance with some embodiments of the technology described herein. The horizontal line in FIG. 24 illustrates an example threshold for determining the existence of an acute attack corresponding to a BK level of 20 pM and a RO of 25.8%.

Having verified the accuracy of the QSP model as described herein, the QSP model may be implemented in a number of different methods for evaluating the effects of HAE on the contact system and for evaluating new and existing treatment modalities for HAE, as will be described further herein.

Sensitivity Analyses

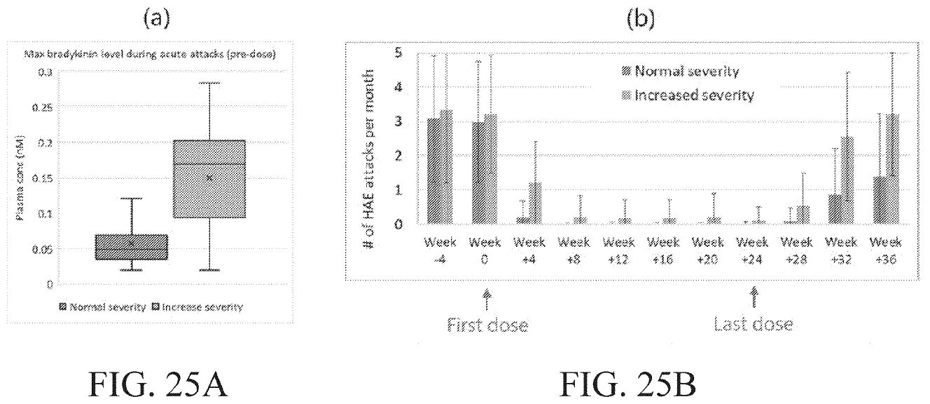

The QSP model, and in particular, the acute attack model, may be used to investigate the sensitivity of monthly attack rates to different parameters, including, for example, attack severity, frequency, and drug binding affinity under a treatment regimen. In the illustrated embodiments, the treatment regimen is 300 mg Q2W lanadelumab, which was modeled over a virtual population of 1000 virtual patients. FIG. 25 illustrates example relationships between monthly attack rates and attack severity in a virtual patient population being treated with lanadelumab, in accordance with some embodiments of the technology described herein. The increase in severity corresponds to the mean BK level of 150 pM, far exceeding typical BK ranges of 15 to 90 pM experienced during an acute attack.

In some embodiments, the QSP model may be used to evaluate the sensitivity of attack frequency to attack severity, as shown in FIGS. 25A-25B. FIGS. 25A-25B illustrates the efficacy of the dosing regimen under a high severity attack. FIG. 25A illustrates a distribution of maximum BK levels during an attack, comparing BK levels of normal severity attacks and BK levels of increased severity attacks. FIG. 25B is a temporal profile of monthly attack rates in HAE patients treated with lanadelumab (with the first dose being administered at week 0 and the last dose being administered at week 24. FIG. 25B illustrates the efficacy of the dosing regimen in suppressing HAE attacks of normal severity as well as HAE attacks of increased severity.

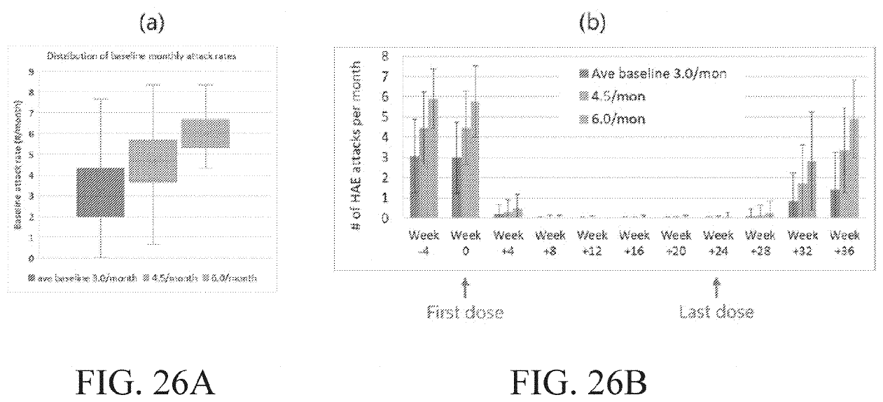

In some embodiments, the QSP model may be used to evaluate the sensitivity of attack frequency to monthly attack rates of untreated patients, as shown in FIGS. 26A-26B. FIGS. 26A-26B illustrate example relationships between monthly attack rates and attack frequency in a virtual patient population being treated with lanadelumab, in accordance with some embodiments of the technology described herein. FIG. 26A illustrates a baseline distribution of monthly HAE acute attacks for different trigger rates (3.0/month, 4.5/month, and 6.0/month). FIG. 26B illustrates a temporal profile of monthly attack rates in a HAE virtual population of 1000 virtual patients treated with a dosage regimen of 300 mg Q2W lanadelumab (with the first dose being administered at week 0 and the last dose being administered at week 24). FIG. 26B illustrates the efficacy of the dosing regimen in suppressing HAE attacks for a range of attack frequencies.

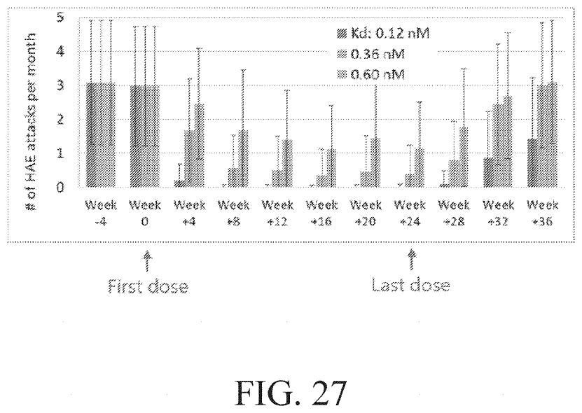

In some embodiments, the QSP model may be used to evaluate the sensitivity of HAE attack frequency to different binding affinities, as shown in FIG. 27. FIG. 27 illustrates an example relationship between monthly attack rates and binding affinity of lanadelumab to kallikrein, in accordance with some embodiments of the technology described herein. FIG. 27 compares the attack frequency for a virtual population of 1000 HAE patients treated with a dosage regimen of 300 mg Q2W lanadelumab (with the first does being administered at week 0 and the last dose being administered at week 24) for different binding affinities (0.12 nM, 0.36 nM, 0.60 nM). FIG. 27 illustrates that stronger binding affinities (e.g., Kd of 0.12 nM) are more effective in reducing HAE attack frequency.

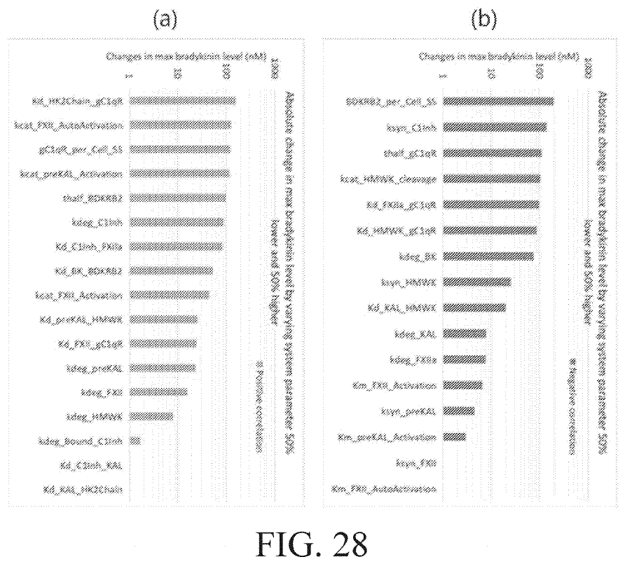

In some embodiments, the QSP model may be used to evaluate the sensitivity of BK level to model parameters of the system, as shown in FIG. 28. FIG. 28 illustrates example relationships of observed bradykinin levels and system model parameters, in accordance with some embodiments of the technology described herein. In particular, FIG. 28 illustrates the change in peak BK level reported in response to varying the model parameter by 100% (50% up and 50% down). The peak BK level shown in FIG. 28 corresponds to the BK level 12 hours after initiation of an acute attack.

FIG. 28a illustrates positive sensitivities of BK level to model parameter variation. FIG. 28b illustrates negative sensitivities of BK level to model parameter variation. For example, an increase in activation rates (kcat_FXII_AutoActivation, kcat_preKAL_Activation) leads to more KAL which in turn leads to more HMWK cleavage, resulting in higher BK level, as expected. An increase in Kd_FXIIa_gC1qR translates to a weaker binding affinity of FXIIa to the receptor, leading to lower KAL activation, lower HMWK cleavage, and resulting in a lower BK level.

Evaluating the sensitivity of peak BK level to model parameters may facilitate development of new treatment modalities which may target different aspects of the contact activation system. For example, the results of the sensitivity analyses described herein may provide insight into the most effective points of the contact activation system for therapeutic intervention.

### Example Model Applications for Evaluating HAE

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

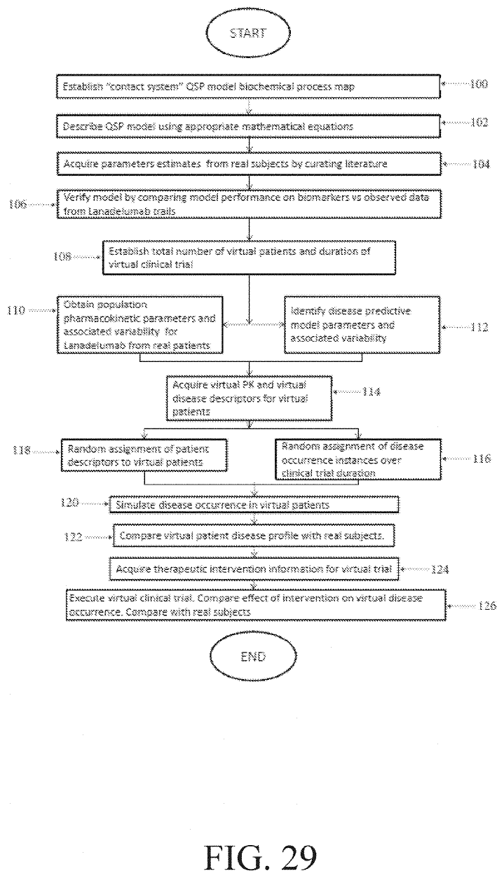

In some embodiments, the QSP model and/or virtual population described herein may be implemented to conduct a virtual clinical trial. FIG. 29 is a flow chart illustrating a computer implemented system and method for modeling, simulating, and evaluating treatments for HAE, in accordance with some embodiments of the technology described herein.

At act 100, a QSP model for modeling a contact system may be established. For example, the QSP model may comprise one or more PK models and/or one or more PD models, as shown in FIG. 2. At act 102, the QSP model may be described with appropriate mathematical equations (e.g., a plurality of ordinary differential equations). In some embodiments, the mathematical equations may describe reactions governing the contact system modeled by the QSP model, for example, as shown in Tables 3a-3b.

At act 104, parameter estimates for parameterizing the QSP model may be acquired from literature and/or clinical data. The parameter estimates may be applied to the QSP model to parameterize the model.

At act 106, the QSP model may be verified by comparing simulation output from the model to literature and/or clinical data. For example, the QSP model may be applied to obtain output for one or more biomarkers (e.g., cHMWK, KAL, BK, etc.), and the output may be compared to biomarker values from clinical data to verify the accuracy of the QSP model.

At act 108, virtual population development may begin by establishing a total number of virtual patients and duration of a virtual clinical trial. For example, in some embodiments, the total number of virtual patients is 1000. The duration of the virtual clinical trial may refer to the length of time the contact system of a patient population is observed, including a time period during which a therapeutic intervention is applied to the patient population.

At acts 110-112, PK parameters and disease predictive descriptors and their associated variabilities may be obtained from real patient data. For example, in some embodiments, clinical data may be used to inform the PK parameters and disease predictive descriptors that are to be applied to the virtual population. At act 114, virtual PK parameters and virtual disease predictive descriptors may be obtained, for example, based on the PK parameters and disease predictive descriptors obtained from clinical data. At acts 116-118, the virtual PK parameters and disease predictive descriptors may be randomly assigned to virtual patients in the virtual patient population.

At act 120, the QSP model may be used to simulate disease occurrence in virtual patients. For example, in some embodiments, the QSP model may be used to simulate occurrence of an acute attack in virtual patients and to reflect the resulting protein levels of the contact activation system. At act 122, the virtual patient disease data may be compared to disease profiles of real subjects with HAE.

At act 124, the QSP model may be used to evaluate the effectiveness of a therapeutic intervention in treating HAE. For example, parameters indicating the virtual patient population is being administered a dosage of a drug (e.g., lanadelumab) according to a dosage regimen may be input into the QSP model.

At act 126, the virtual clinical trial may be executed. For example, the resulting effect of administration of the drug applied in act 124 on the contact system may be observed. In some embodiments, protein levels of the contact system may be evaluated, to determine a relative change in protein levels resulting from administration of the therapeutic intervention. In some embodiments, a characteristic of an acute attack (e.g., attack frequency, attack severity, attack duration, etc.) may be observed. In some embodiments, the virtual clinical trial data may be compared with data from real subjects.

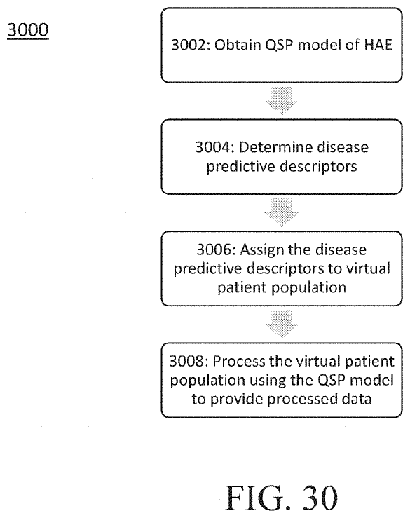

In some embodiments, the QSP model may be used to evaluate the effects of HAE on the contact activation system, as shown in FIG. 30. FIG. 30 illustrates an example method 3000 for modeling and simulating HAE, in accordance with some embodiments of the technology described herein.

Method 3000 begins at act 3002 where a QSP model of HAE is obtained, for example, using any of the techniques for developing, parameterizing, and/or verifying a QSP model described herein. The QSP model may comprise one or more PK models and/or one or more PD models, as shown in FIG. 2. In some embodiments, QSP model may comprise a plurality of ordinary differential equations. In some embodiments, the mathematical equations may describe reactions governing the contact system modeled by the QSP model, for example, as shown in Tables 3a-3b.

At act 3004, disease predictive descriptors may be obtained. For example, disease predictive descriptors may include a virtual patient's propensity to experience an acute attack, for example, attack frequency, attack severity, and/or attack duration. In some embodiments, the disease predictive descriptors, for example, attack frequency, are determined at least in part by a Poisson process informed by known data regarding the disease predictive descriptors.

At act 3006, the disease predictive descriptors may be assigned to a data set. For example, the data set may represent a virtual patient population for which the QSP model is applied. The virtual population may comprise a plurality of data sets. Each data set (e.g., Patients) may represent an individual virtual patient of the virtual population and may have one or more variables (e.g., for assigning PK parameters and/or disease predictive descriptors) defining one or more characteristics of the virtual patient.

At act 3008, the data set may be processed using the QSP model (e.g., by inputting the data set to the QSP model) to obtain processed data. The processed data may include, for example, protein levels of the contact system for a virtual patient. In some embodiments, the method further comprises displaying the processed data.

In some embodiments, the method further comprises determining and assigning PK parameters for the data set, and determining the effectiveness of a therapeutic intervention by processing therapeutic intervention data and the data set with the QSP model. For example, in some embodiments, the therapeutic intervention comprises administering lanadelumab. In some embodiments, the therapeutic intervention comprises administering a small molecule PKa inhibitor (e.g., orally). In some embodiments, determining the effectiveness of the therapeutic intervention comprises evaluating protein levels of the contact activation system, provided by the QSP model, as a result of administering the therapeutic intervention.

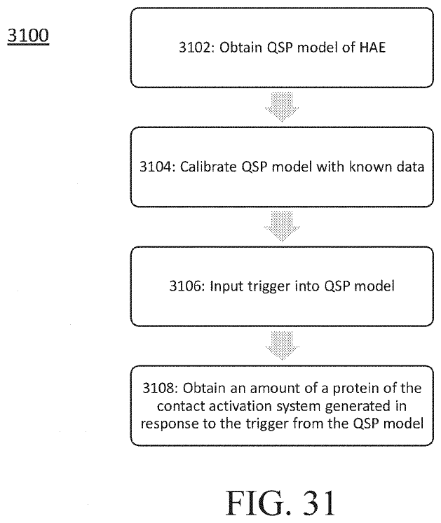

In some embodiments, the QSP model may be used to estimate one or more characteristics of a contact system in response to a trigger, as shown in FIG. 31. The example method of FIG. 31, method 3100, begins at act 3102, where a QSP model of HAE is obtained. At act 3104, the QSP model may be calibrated (e.g., parameterized) with known data, for example, known data from one or more clinical trials.

At act 3106, a trigger may be input into the QSP model. For example, the trigger may be a signal input into the QSP model causing Factor XII to autoactivate to generate Factor XIIa.

At act 3108, an amount of a protein (e.g., BK, KAL, cHMWK, etc.) of the contact system generated in response to the trigger may be obtained. In some embodiments, the amount of the protein may be compared to a known amount of the protein (e.g., obtained from clinical data), to, for example, determine whether an acute attack has occurred in response to the trigger. In some embodiments, the amount of the protein may be used to determine the severity and/or duration of an acute attack occurring in response to the trigger.

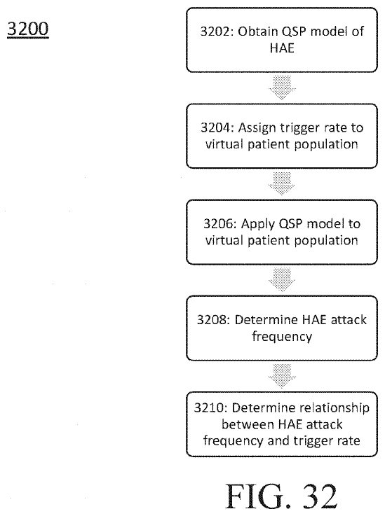

In some embodiments, the QSP model may be used to determine a relationship between HAE attack frequency and Factor XII trigger rate. For example, FIG. 32 illustrates an example method 3200 for determining a relationship between HAE attack frequency and a trigger rate for autoactivation of Factor XII, in accordance with some embodiments of the technology described herein. Method 3200 begins at act 3202 where a QSP model of HAE is obtained, for example, according to any of the techniques described herein.

At act 3204, a trigger rate for FXII autoactivation is assigned to a virtual population. For example, each patient in the virtual population may be assigned a trigger rate. In some embodiments, one or more different trigger rates may be assigned to the virtual population such that not all patients are assigned the same trigger rate. In some embodiments, the trigger rate(s) assigned to the virtual population are based on clinical data (e.g., trigger rates of HAE patients obtained from one or more clinical trials). In some embodiments, the trigger rate(s) may be assigned to the virtual population using a Poisson distribution.

At act 3206, the QSP model is applied to the virtual population. For example, the virtual population data with assigned trigger rates may be input into the QSP model to obtain information about contact system protein levels for each patient in the virtual population.

At act 3208, an HAE attack frequency for the virtual population may be obtained from the QSP model. For example, protein levels obtained from the QSP model may be used to determine the occurrence and frequency of an acute attack. At act 3210, a relationship between HAE attack frequency and trigger rate is determined. For example, the FXII autoactivation trigger rate may be compared to the HAE attack frequency. In some embodiments, the relationship between HAE attack frequency and trigger rate may reflect the frequency in which FXII autoactivation results in an HAE attack.

### Example Model Applications for Evaluating Therapeutic Interventions

As described herein, the QSP model may be used to evaluate the effectiveness of new or existing therapeutic interventions for treating HAE. The inventors have recognized that use of the QSP model to evaluate new or existing therapeutic interventions may be advantageous, as it provides for more rapid evaluation when compared to a clinical trial, and allows for evaluation of new treatment modalities before testing such treatment modalities on a human patient. In addition, the QSP model may provide more accurate evaluation of new or existing treatment modalities as use of the QSP model described in the present application may provide various types of information about the contact system in a patient which would be impractical or impossible to clinically obtain.

Evaluating Effectiveness of New or Existing Drugs for Treating HAE

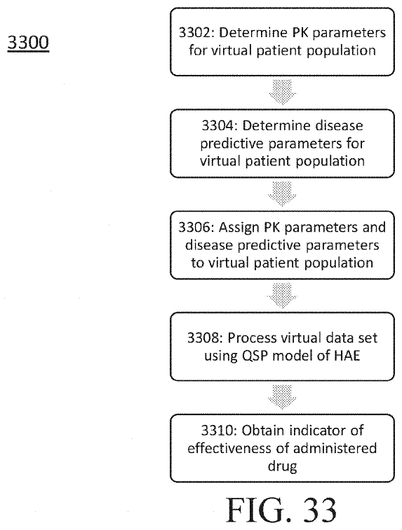

In some embodiments, the QSP model may be used to evaluate the effectiveness of new or existing drugs for treating HAE. FIG. 33 illustrates an example method 3300 for determining an effectiveness of an administered drug in treating HAE, in accordance with some embodiments of the technology described herein.

Method 3300 begins at act 3302 where PK parameters for a virtual data set may be obtained. As described herein, the PK parameters may be used to describe the disposition of a drug in a patient. The virtual data set may reflect a virtual patient population on which the virtual clinical trial executed by the QSP model is run. The dosage and characteristics of the drug administered to each virtual patient may be reflected by the PK parameters.

At act 3304, disease predictive descriptors (e.g., attack frequency, severity, duration, etc.) may be determined for the virtual data set. In some embodiments, the disease predictive descriptors may be informed by clinical data.

At act 3306, the PK parameters and disease predictive descriptors are assigned to the virtual data set. In some embodiments, the disease predictive descriptors may be assigned using a Poisson process.

At act 3308, the virtual data set may be processed by a QSP model to obtain processed data. At act 3310, an indicator of the effectiveness of the administered drug may be obtained. In some embodiments, the processed data output by the QSP model may include one or more levels of contact system proteins (e.g., BK, cHMWK, KAL, etc.). The protein levels may be used to determine the effectiveness of the administered drug. For example, reduced levels of BK, cHMWK, and KAL may indicate the drug is effectively inhibiting HAE attacks. In some embodiments, the protein levels obtained from the QSP model may be used to determine a characteristic of an HAE acute attack (e.g., attack frequency, severity, and/or duration). In some embodiments, the acute attack characteristics may be used to determine an effectiveness of the administered drug (for example, by observing a reduction in acute attack frequency).

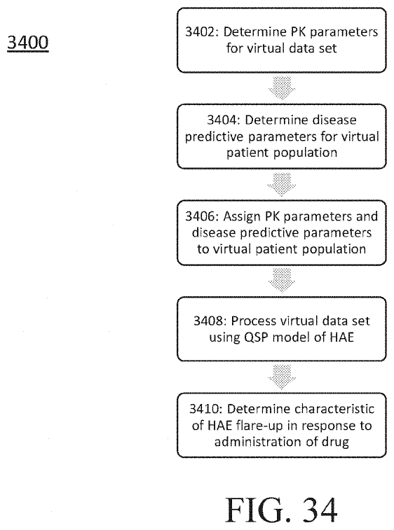

More particularly, in some embodiments, the QSP model may be used to determine a characteristic of an HAE flare-up (e.g., attack frequency, severity, duration, etc.) in a patient in response to receiving treatment. FIG. 34 illustrates a method 3400 for determining a characteristic of an HAE flare-up in response to administering a drug to a patient, in accordance with some embodiments of the technology described herein.

Method 3400 beings at act 3402 where PK parameters for a virtual data set may be obtained. As described herein, the PK parameters may be used to describe the disposition of a drug in a patient. The virtual data set may reflect a virtual patient population on which the virtual clinical trial executed by the QSP model is run. The dosage and characteristics of the drug administered to each virtual patient may be reflected by the PK parameters.

At act 3404, disease predictive descriptors (e.g., attack frequency, severity, duration, etc.) may be determined for the virtual data set. In some embodiments, the disease predictive descriptors may be informed by clinical data.

At act 3406, the PK parameters and disease predictive descriptors are assigned to the virtual data set. In some embodiments, the disease predictive descriptors may be assigned using a Poisson process.

At act 3408, the virtual data set may be processed by a QSP model to obtain processed data. At act 3410, one or more characteristics of an HAE flare-up in response to administration of a drug may be determined. For example, in some embodiments, characteristics of the HAE flare-up may include attack frequency, attack severity, and/or attack duration. In some embodiments, the one or more characteristics of the HAE flare-up may be used to determine the effectiveness of the administered drug, for example, by comparing the one or more characteristics of the HAE flare-up to known data. For example, HAE attack frequency obtained from the QSP model for the virtual population of patients receiving treatment may be compared to HAE attack frequency in untreated patients to determine if the administered drug reduces HAE attack frequency.

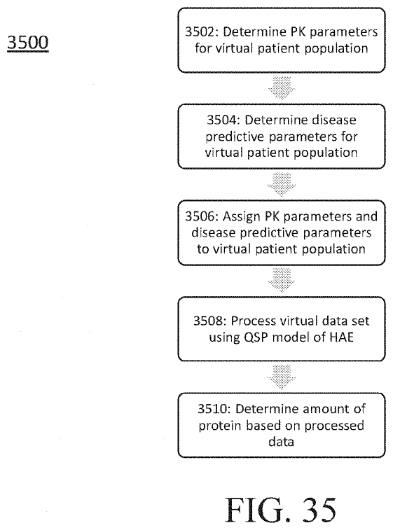

In some embodiments, the QSP model may be used to determine a protein level of the contact system of a patient in response to receiving treatment. FIG. 35 illustrates an example method 3500 for determining an amount of a protein of a contact system in a patient in response to administration of a drug for treating HAE, in accordance with some embodiments of the technology described herein.

Method 3500 beings at act 3502 where PK parameters for a virtual data set may be obtained. As described herein, the PK parameters may be used to describe the disposition of a drug in a patient. The virtual data set may reflect a virtual patient population on which the virtual clinical trial executed by the QSP model is run. The dosage and characteristics of the drug administered to each virtual patient may be reflected by the PK parameters.

At act 3504, disease predictive descriptors (e.g., attack frequency, severity, duration, etc.) may be determined for the virtual data set. In some embodiments, the disease predictive descriptors may be informed by clinical data.

At act 3506, the PK parameters and disease predictive descriptors are assigned to the virtual data set. In some embodiments, the disease predictive descriptors may be assigned using a Poisson process.

At act 3508, the virtual data set may be processed by a QSP model to obtain processed data. At act 3510, an amount of a protein of the contact system may be determined based on the processed data. In particular, the QSP model may produce, as output, a protein level of one or more proteins of the contact system (e.g., cHMWK, BK, KAL, etc.). In some embodiments, an effectiveness of an administered drug may be determined based on relative changes in protein levels. For example, reductions in amounts of certain proteins of the contact system (e.g., cHMWK, BK, KAL, etc.) in treated patients as compared to untreated HAE patients may indicate the administered drug is effectively inhibiting acute HAE attacks. Therefore, in some embodiments, the levels of the one or more proteins of virtual patients receiving treatment for HAE may be compared with known data of protein levels of untreated HAE patients.

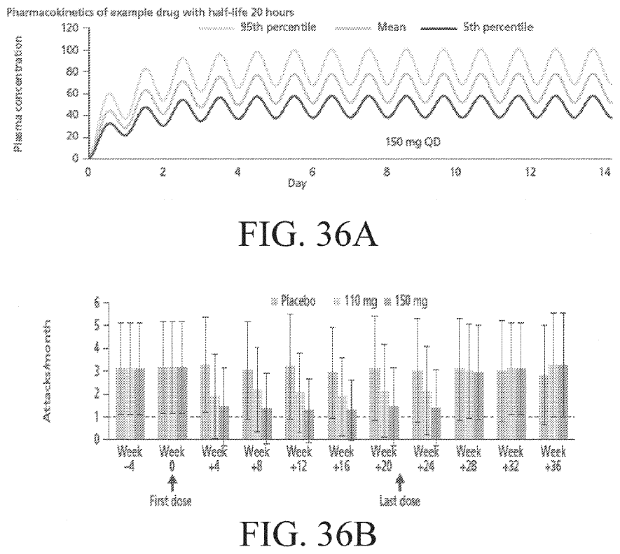

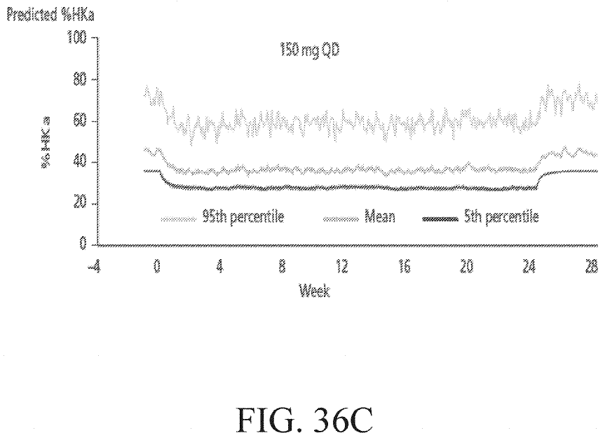

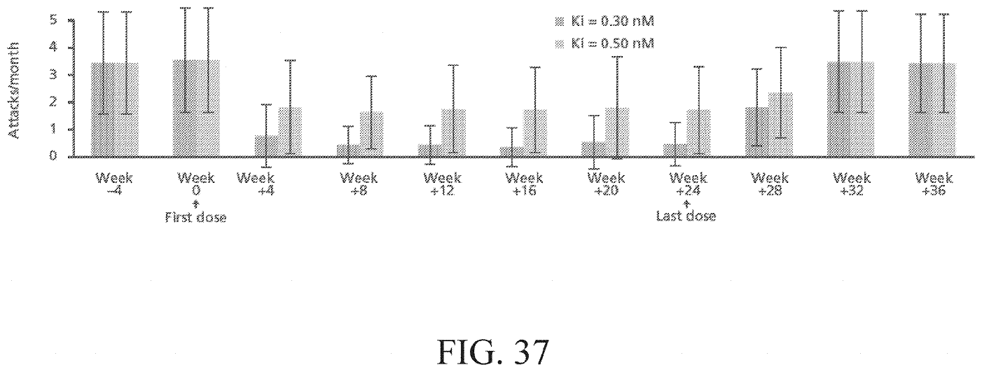

FIGS. 36A-37 illustrate example results obtained from embodiments of the methods described herein. FIGS. 36A-36C illustrate example relationships between drug effectiveness in treating HAE and binding affinity, and half-life, in accordance with some embodiments of the technology described herein. FIG. 36A illustrates PK parameters, more specifically, plasma concentrations, of a small molecule PKA inhibitor having a half-life of 20 hours. FIG. 36B illustrates simulation results of attack frequency in patients treated with 110 mg and 150 mg QD of the small molecule PKA inhibitor. A placebo group was also tested for comparison. As shown in FIG. 36B, a higher dosage (150 mg QD) of the small molecule PKA inhibitor was more effective in reducing attack frequency in virtual patients with HAE.

FIG. 36C illustrates simulation results of protein levels, more specifically, percentage cHMWK (% HKa) in virtual patients being administered 150 mg QD of the small molecule PKA inhibitor. Compared with results from lanadelumab, having a stronger binding affinity and longer half-life of 14 days, the small molecule PKA was less effective at reducing attack frequency and percentage cHMWK amounts. The simulation results suggest that drugs having a stronger binding affinity and longer half-life, such as lanadelumab, are more effective in treating HAE.

FIG. 37 illustrates an example relationship of monthly attack rates and inhibitions constants of administered drugs, in accordance with some embodiments of the technology described herein. FIG. 37 illustrates an example of using the QSP model to evaluate the effect of drug characteristics on effectiveness of the drug in treating HAE. In particular, FIG. 37 illustrates simulation results for attack frequency for drugs with different binding affinities (0.30 nM and 0.50 nM). As seen in FIG. 37, the stronger binding affinity (0.30 nM) is more effective at reducing HAE attack frequency than the weaker binding affinity (0.50 nM). As shown in FIGS. 36A-37, simulation results from the QSP model may be used to inform development of new and/or existing treatment modalities for HAE.

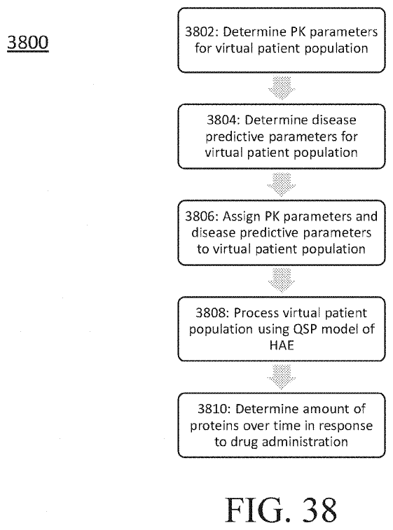

In some embodiments, the QSP model may be used for determining a temporal profile of a drug's effect on HAE. For example, FIG. 38 illustrates an example method 3800 for determining a temporal profile illustrating an effect of a drug on a contact system in a patient, in accordance with some embodiments of the technology described herein.

Method 3800 beings at act 3802 where PK parameters for a virtual data set may be obtained. As described herein, the PK parameters may be used to describe the disposition of a drug in a patient. The virtual data set may reflect a virtual patient population on which the virtual clinical trial executed by the QSP model is run. The dosage and characteristics of the drug administered to each virtual patient may be reflected by the PK parameters.

At act 3804, disease predictive descriptors (e.g., attack frequency, severity, duration, etc.) may be determined for the virtual data set. In some embodiments, the disease predictive descriptors may be informed by clinical data.

At act 3806, the PK parameters and disease predictive descriptors are assigned to the virtual data set. In some embodiments, the disease predictive descriptors may be assigned using a Poisson process.

At act 3808, the virtual data set may be processed by a QSP model to obtain processed data. At act 3810, amounts of proteins of the contact system may be obtained over a period of time. For example, in some embodiments, an amount of a protein (e.g., cHMWK, BK, KAL, etc.) may be obtained at different points in time to map a change in the amount of the protein over time. The change in protein amount over time may be used to determine an effectiveness of an administered drug. For example, levels of certain proteins (e.g., cHMWK, BK, KAL, etc.) showing little to no change over time may indicate that the administered drug is effectively inhibiting HAE flare-ups.

Evaluating Efficacy of Combination Therapies for Treating HAE

In some embodiments, the QSP model may be used to evaluate the effectiveness of combination therapies for treating HAE. For example, in some embodiments, a patient may be administered two or more drugs for treating HAE. The methods described herein for using the QSP model to evaluate the effectiveness of a drug may likewise be applied to evaluate the effectiveness of a combination therapy.

Evaluating Efficacy of Dosages

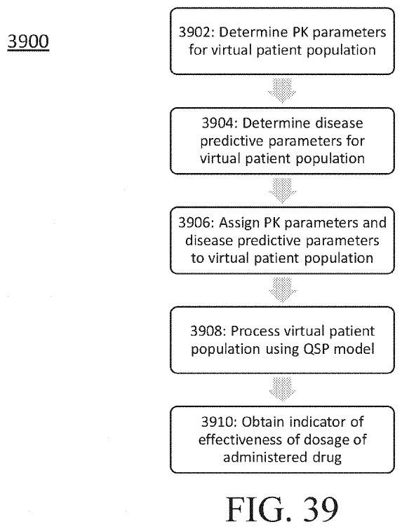

In some embodiments, the QSP model may be used to evaluate the effectiveness of a particular dosage of an administered drug. For example, FIG. 39 illustrates an example method 3900 for determining an effectiveness of a dosage of an administered drug in treating HAE, in accordance with some embodiments of the technology described herein.

Method 3900 beings at act 3902 where PK parameters for a virtual data set may be obtained. As described herein, the PK parameters may be used to describe the disposition of a drug in a patient. The virtual data set may reflect a virtual patient population on which the virtual clinical trial executed by the QSP model is run. The dosage and characteristics of the drug administered to each virtual patient may be reflected by the PK parameters.

At act 3904, disease predictive descriptors (e.g., attack frequency, severity, duration, etc.) may be determined for the virtual data set. In some embodiments, the disease predictive descriptors may be informed by clinical data.

At act 3906, the PK parameters and disease predictive descriptors are assigned to the virtual data set. In some embodiments, the disease predictive descriptors may be assigned using a Poisson process.

At act 3908, the virtual data set may be processed by a QSP model to obtain processed data. At act 3910, an indicator of the effectiveness of a dosage of an administered drug may be obtained. For example, the simulation output may provide levels of one or more proteins, including changes in protein level over time, and/or one or more characteristics of an HAE flare-up (e.g., attack frequency, severity, duration, etc.). The simulation output may be used as described herein for determining the effectiveness of the dosage of the administered drug input into the QSP model.

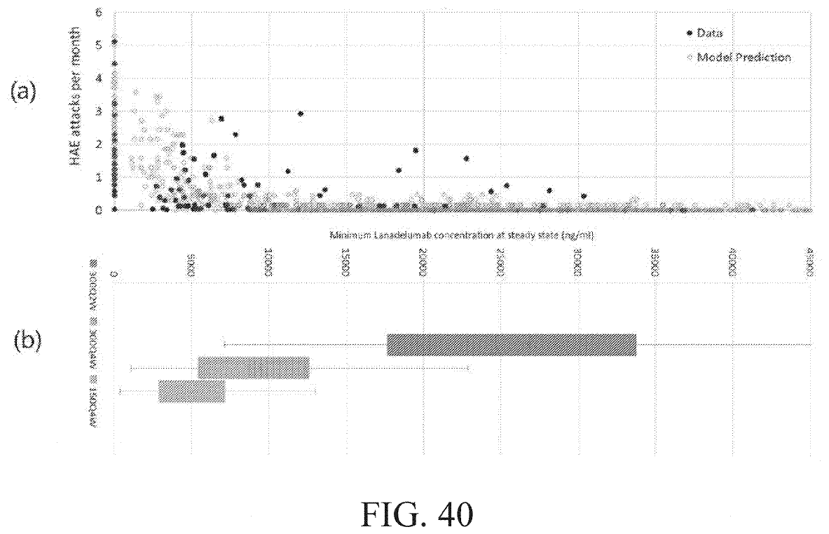

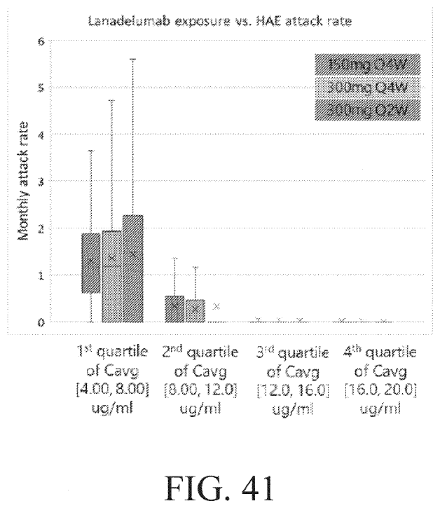

FIGS. 40-41 illustrate results of embodiments of the methods described herein for determining the effectiveness of a dosage of an administered drug. FIG. 40 illustrates example relationships of drug exposure and HAE attack response, in accordance with some embodiments of the technology described herein. In particular, graph (a) compares HAE attack frequency with concentration of lanadelumab in a virtual population. Graph (b) illustrates ranges of concentrations of lanadelumab in the virtual population achieved for particular dosages (300 mg Q2W, 300 mg Q4W, and 150 mg Q4W) according to the PK model.

FIG. 41 further illustrates an example relationship drug exposure and HAE attack response, in accordance with some embodiments of the technology described herein. In particular, FIG. 41 segregates the HAE attack frequency into quartiles for different concentrations of the administered drug. The results from FIGS. 40-41 illustrate that higher dosages (and therefore higher concentrations) of lanadelumab were more effective at treating HAE than lower dosages (and therefore lower concentrations), however, the effectiveness of higher dosages reaches diminishing returns at a concentration of about 12 μg/ml.

Evaluating Efficacy of Dosage Frequencies and/or Dosage Regimens

In some embodiments, the QSP model may be used to evaluate the effectiveness of a particular dosage frequency and/or dosage regimen (for example, evaluating the manner in which a dose is applied, e.g., orally, etc.). The methods described herein for using the QSP model to evaluate the effectiveness of a drug may likewise be applied to evaluate the effectiveness of a dosage frequency and/or dosage regimen.

Evaluating the Effect of Non-Adherence to a Dosage Schedule

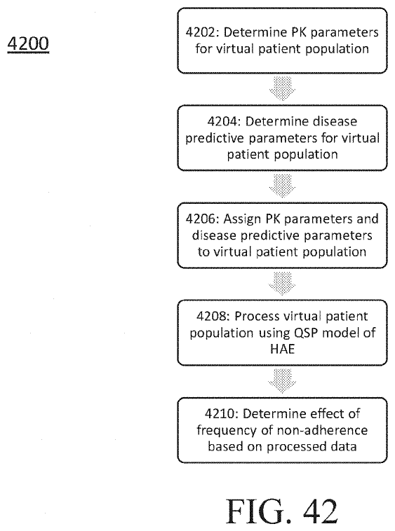

In some embodiments, the QSP model may be used to evaluate the effect of non-adherence to a dosage schedule (e.g., missing one or more scheduled dosages). For example, FIG. 42 illustrates an example method for determining an effect of non-adherence to a dosing regimen of an administered drug in treating HAE, in accordance with some embodiments of the technology described herein.

Method 4200 beings at act 4202 where PK parameters for a virtual data set may be obtained. As described herein, the PK parameters may be used to describe the disposition of a drug in a patient. The virtual data set may reflect a virtual patient population on which the virtual clinical trial executed by the QSP model is run. The dosage and characteristics of the drug administered to each virtual patient may be reflected by the PK parameters. In particular, the PK parameters may reflect one or more missed dosages according to the method 4200.

At act 4204, disease predictive descriptors (e.g., attack frequency, severity, duration, etc.) may be determined for the virtual data set. In some embodiments, the disease predictive descriptors may be informed by clinical data.

At act 4206, the PK parameters and disease predictive descriptors are assigned to the virtual data set. In some embodiments, the disease predictive descriptors may be assigned using a Poisson process.

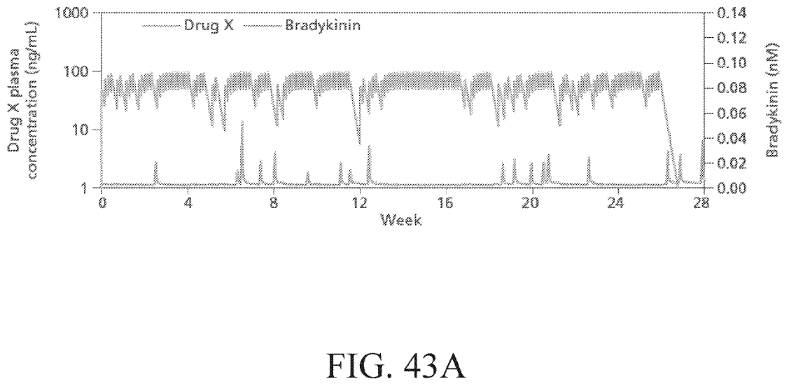

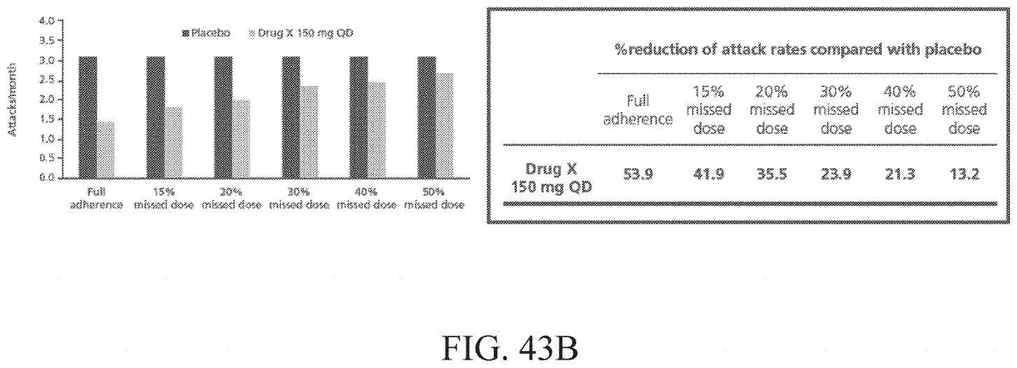

At act 4208, the virtual data set may be processed by a QSP model to obtain processed data. At act 4210, an effect of non-adherence (including non-adherence frequency) may be determined. For example, the simulation output may provide levels of one or more proteins, including changes in protein level over time, and/or one or more characteristics of an HAE flare-up (e.g., attack frequency, severity, duration, etc.). The simulation output may be used as described herein for determining the effect of missing one or more scheduled dosages, as shown in FIG. 43A, for example. In some embodiments, the effects of different frequencies of non-adherence (e.g., full adherence, 15% missed dose, 20% missed dose, etc.) may be compared to determine the effects of non-adherence on HAE treatment, as shown in FIG. 43B, for example.

FIG. 43A illustrates an example relationship between nonadherence to a dosage regimen and bradykinin levels, in accordance with some embodiments of the technology described herein. In particular, FIG. 43A illustrates BK levels for a virtual patient administered 150 mg QD of a drug for treating HAE with a 20% rate of non-adherence. As shown in FIG. 43A, BK levels increase after days in which concentration of the administered drug decreases (due to a missed dosage). FIG. 43A therefore illustrates that non-adherence to the daily dosage regimen may negatively impact suppression of HAE attacks as each missed dose reduces drug coverage and makes the patient more prone to HAE attacks.

FIG. 43B illustrates examples relationships between nonadherence rates and attack frequency, in accordance with some embodiments of the technology described herein. In particular, FIG. 43B illustrates an increase in attack frequency as non-adherence rates increase. The percentage reduction of HAE attacks reduces from 53.9% at full adherence to 13.2% at 50% missed doses. More missed doses results in higher HAE attack frequency, with 50% missed dose scenarios resulting in marginal drug efficacy.

### CONCLUSION

Having thus described several aspects of at least one embodiment, it is to be appreciated that various alterations, modifications, and improvements will readily occur to those skilled in the art. Such alterations, modifications, and improvements are intended to be within the spirit and scope of the present disclosure. Accordingly, the foregoing description and drawings are by way of example only.

For example, in some embodiments, the contact system may be modified and/or used to model one or more other diseases other than HAE, for example other diseases which implicate the contact system or similar biological systems (e.g., other diseases resulting in edemas).

In addition, although the QSP model has been described herein for evaluating HAE treatments which inhibit the kinin-kallikrein cascade, in some embodiments, the QSP model may be used to evaluate other HAE treatments which impact other parts of the contact system, for example, FXIIa inhibitors and/or enzymes which function to degrade BK.

The above-described embodiments of the present disclosure can be implemented in any of numerous ways. For example, the embodiments may be implemented using hardware, software or a combination thereof. When implemented in software, the software code can be executed on any suitable processor or collection of processors, whether provided in a single computer or distributed among multiple computers.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the concepts disclosed herein may be embodied as a non-transitory computer-readable medium (or multiple computer-readable media) (e.g., a computer memory, one or more floppy discs, compact discs, optical discs, magnetic tapes, flash memories, circuit configurations in Field Programmable Gate Arrays or other semiconductor devices, or other non-transitory, tangible computer storage medium) encoded with one or more programs that, when executed on one or more computers or other processors, perform methods that implement the various embodiments of the present disclosure discussed above. The computer-readable medium or media can be transportable, such that the program or programs stored thereon can be loaded onto one or more different computers or other processors to implement various aspects of the present disclosure as discussed above.

The terms “program” or “software” are used herein to refer to any type of computer code or set of computer-executable instructions that can be employed to program a computer or other processor to implement various aspects of the present disclosure as discussed above. Additionally, it should be appreciated that according to one aspect of this embodiment, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various features and aspects of the present disclosure may be used alone, in any combination of two or more, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the concepts disclosed herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The terms “substantially”, “approximately”, and “about” may be used to mean within ±20% of a target value in some embodiments, within ±10% of a target value in some embodiments, within ±5% of a target value in some embodiments, within ±2% of a target value in some embodiments. The terms “approximately” and “about” may include the target value.

Use of ordinal terms such as “first,” “second,” “third,” etc. in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

What is claimed is:

1. A computer-implemented method for modeling and simulating hereditary angioedema (HAE), comprising:
obtaining a quantitative systems pharmacology (QSP) model of HAE, wherein the QSP model is configured to represent autoactivation of Factor XII by elevating levels of FXIIa in response to an indication that a trigger has been input into the QSP model;
determining disease predictive descriptors;
assigning the disease predictive descriptors to a virtual patient population; and
processing the virtual patient population using the QSP model to provide processed data, wherein the processed data comprises an amount of one or more contact system proteins.

2. The computer-implemented method of claim 1, further comprising displaying the processed data.

3. The computer-implemented method of claim 1 or any other preceding claim, further comprising:
determining pharmacokinetic parameters;
assigning the pharmacokinetic parameters to the virtual patient population;
determining therapeutic intervention data based on a therapeutic intervention; and
processing the therapeutic intervention data and the virtual patient population with the QSP model to determine effectiveness of a therapeutic intervention.

4. The computer-implemented method of claim 2 or any other preceding claim, wherein the therapeutic intervention comprises administering lanadelumab.

5. The computer-implemented method of claim 2 or any other preceding claim, wherein the therapeutic intervention comprises administering a small molecule PKa inhibitor.

6. The computer-implemented method of claim 2 or any other preceding claim, wherein the therapeutic intervention comprises administering the small molecule PKa inhibitor orally.

7. The computer-implemented method of claim 1 or any other preceding claim, wherein the one or more contact system proteins comprise at least one of bradykinin, cHMWK, or plasma kallikrein.

8. The computer-implemented method of claim 1 or any other preceding claim, further comprising using the processed data to determine an HAE flare-up frequency.

9. The computer-implemented method of claim 1 or any other preceding claim, further comprising using the processed data to determine an HAE flare-up severity.

10. The computer-implemented method of claim 1 or any other preceding claim, further comprising using the processed data to determine an HAE flare-up duration.

11. The computer-implemented method of claim 1 or any other preceding claim, wherein the QSP model comprises a plurality of differential equations representing one or more biological reactions of a contact system.

12. The computer-implemented method of claim 3 or any other preceding claim, wherein the pharmacokinetic parameters comprise one or more parameters indicating how the therapeutic intervention is impacted by one or more biographical characteristics of a patient to whom the therapeutic intervention is administered.

13. The computer-implemented method of claim 12 or any other preceding claim, wherein the one or more biographical characteristics comprise at least one of height, weight, age, or gender.

14. The computer-implemented method of claim 1 or any other preceding claim, wherein the disease predictive descriptors comprise one or more parameters characterizing a propensity of a patient to experience an HAE flare-up.

15. The computer-implemented method of claim 14 or any other preceding claim, wherein the disease predictive descriptors include HAE flare-up frequency and/or severity.

16. A system, comprising:
at least one computer hardware processor; and
at least one non-transitory computer-readable storage medium storing processor executable instructions that, when executed by the at least one computer hardware processor, cause the at least one computer hardware processor to perform a computer-implemented method for modeling and simulating hereditary angioedema (HAE), comprising:
obtaining a quantitative systems pharmacology (QSP) model of HAE, wherein the QSP model is configured to represent autoactivation of Factor XII by elevating levels of FXIIa in response to an indication that a trigger has been input into the QSP model;
determining disease predictive descriptors;
assigning the disease predictive descriptors to a virtual patient population; and
processing the virtual patient population using the QSP model to provide processed data, wherein the processed data comprises an amount of one or more contact system proteins.

17. At least one non-transitory computer-readable medium storing processor executable instructions that, when executed by at least one computer hardware processor, cause the at least one computer hardware processor to perform a computer implemented method for modeling and simulating hereditary angioedema (HAE), the comprising:
obtaining a quantitative systems pharmacology (QSP) model of HAE, wherein the QSP model is configured to represent autoactivation of Factor XII by elevating levels of FXIIa in response to an indication that a trigger has been input into the QSP model;
determining disease predictive descriptors;
assigning the disease predictive descriptors to a virtual patient population; and
processing the virtual patient population using the QSP model to provide processed data, wherein the processed data comprises an amount of one or more contact system proteins.

18. A computer-implemented method for determining a trigger strength by estimating one or more characteristics of a contact system in a patient in response to a trigger, the method comprising:
obtaining a quantitative systems pharmacology (QSP) model of hereditary angioedema (HAE), wherein the QSP model is configured to represent autoactivation of Factor XII by elevating levels of FXIIa in response to an indication that the trigger has been input into the QSP model;
calibrating the QSP model with known data;
inputting the trigger into the QSP model, the trigger being configured to generate FXIIa by causing Factor XII of the contact system to autoactivate;
obtaining, from the QSP model, an amount of a protein of the contact system generated in response to the trigger.

19. The computer-implemented method of claim 18, further comprising comparing the amount of the protein to a known amount of the protein obtained from clinical data.

20. The computer-implemented method of claim 18 or any other preceding claim, further comprising using the amount of the protein to determine whether an HAE flare-up has occurred in response to the trigger.

21. The computer-implemented method of claim 20 or any other preceding claim, further comprising using the amount of the protein to determine the severity of the HAE flare-up.

22. The computer-implemented method of claim 20 or any other preceding claim, wherein:
the protein comprises bradykinin; and
using the amount of the protein to determine whether an HAE flare-up has occurred comprises determining whether the amount of bradykinin exceeds a threshold.

23. The computer-implemented method of claim 20 or any other preceding claim, further comprising using the amount of the protein to determine the duration of the HAE flare-up.

24. The computer-implemented method of claim 18 or any other preceding claim, wherein the protein is bradykinin.

25. The computer implemented method of claim 18 or any other preceding claim, wherein the protein is cHMWK.

26. The computer-implemented method of claim 18 or any other preceding claim, wherein the protein is plasma kallikrein.

27. The computer-implemented method of claim 18 or any other preceding claim, wherein the QSP model comprises a plurality of differential equations representing one or more biological reactions of the contact system.

28. A system, comprising:
at least one computer hardware processor; and
at least one non-transitory computer-readable storage medium storing processor executable instructions that, when executed by the at least one computer hardware processor, cause the at least one computer hardware processor to perform a computer-implemented method for estimating one or more characteristics of a contact system in a patient in response to a trigger, the method comprising:
obtaining a quantitative systems pharmacology (QSP) model of hereditary angioedema (HAE), wherein the QSP model is configured to represent autoactivation of Factor XII by elevating levels of FXIIa in response to an indication that the trigger has been input into the QSP model;
calibrating the QSP model with known data;
inputting the trigger into the QSP model, the trigger being configured to generate FXIIa by causing Factor XII of the contact system to autoactivate;
obtaining, from the QSP model, an amount of a protein of the contact system generated in response to the trigger.

29. At least one non-transitory computer-readable storage medium storing processor executable instructions that, when executed by at least one computer hardware processor, cause the at least one computer hardware processor to perform a computer-implemented method for estimating one or more characteristics of a contact system in a patient in response to a trigger, the method comprising:
obtaining a quantitative systems pharmacology (QSP) model of hereditary angioedema (HAE), wherein the QSP model is configured to represent autoactivation of Factor XII by elevating levels of FXIIa in response to an indication that the trigger has been input into the QSP model;
calibrating the QSP model with known data;
inputting the trigger into the QSP model, the trigger being configured to generate FXIIa by causing Factor XII of the contact system to autoactivate;
obtaining, from the QSP model, an amount of a protein of the contact system generated in response to the trigger.

30. A computer-implemented method for determining a relationship between hereditary angioedema (HAE) attack frequency and Factor XII trigger rate, the method comprising:
obtaining a quantitative systems pharmacology (QSP) model of HAE, wherein the QSP model is configured to represent autoactivation of Factor XII by elevating levels of FXIIa in response to an indication that a trigger has been input into the QSP model;
assigning a Factor XII trigger rate for one or more patients in a virtual patient population, wherein the Factor XII trigger rate comprises a rate at which autoactivation of Factor XII is triggered in the QSP model;
applying the QSP model to the one or more patients in the virtual patient population to obtain processed data, wherein the processed data comprises an amount of one or more contact system proteins;
determining an HAE attack frequency for the one or more patients in the virtual patient population based on the processed data; and
determining a relationship between HAE attack frequency and Factor XII trigger rate.

31. The computer-implemented method of claim 30, further comprising obtaining an amount of bradykinin generated in response to the autoactivation of Factor XII.

32. The computer-implemented method of claim 30 or any other preceding claim, further comprising obtaining an amount of cHMWK in response to the autoactivation of Factor XII.

33. The computer-implemented method of claim 30 or any other preceding claim, further comprising obtaining an amount of plasma kallikrein generated in response to the autoactivation of Factor XII.

34. The computer-implemented method of claim 30 or any other preceding claim, further comprising calibrating the QSP model with known data.

35. The computer-implemented method of claim 30 or any other preceding claim, further comprising verifying the QSP model at least in part by comparing the determined HAE attack frequency for the one or more patients in the virtual patient population with known data.

36. The computer-implemented method of claim 30 or any other preceding claim, wherein the QSP model comprises a plurality of differential equations representing one or more biological reactions of a contact system.

37. A system, comprising:
at least one computer hardware processor; and
at least one non-transitory computer-readable storage medium storing processor executable instructions that, when executed by at least one computer hardware processor, cause the at least one computer hardware processor to perform a computer-implemented method for determining a relationship between hereditary angioedema (HAE) attack frequency and Factor XII trigger rate, the method comprising
obtaining a quantitative systems pharmacology (QSP) model of HAE, wherein the QSP model is configured to represent autoactivation of Factor XII by elevating levels of FXIIa in response to an indication that a trigger has been input into the QSP model;
assigning a Factor XII trigger rate for one or more patients in a virtual patient population, wherein the Factor XII trigger rate comprises a rate at which autoactivation of Factor XII is triggered in the QSP model;
applying the QSP model to the one or more patients in the virtual patient population to obtain processed data, wherein the processed data comprises an amount of one or more contact system proteins;
determining an HAE attack frequency for the one or more patients in the virtual patient population based on the processed data; and
determining a relationship between HAE attack frequency and Factor XII trigger rate.

38. At least one non-transitory computer-readable storage medium storing processor executable instructions that, when executed by at least one computer hardware processor, cause the at least one computer hardware processor to perform a computer-implemented method for determining a relationship between hereditary angioedema (HAE) attack frequency and Factor XII trigger rate, the method comprising:
obtaining a quantitative systems pharmacology (QSP) model of HAE, wherein the QSP model is configured to represent autoactivation of Factor XII by elevating levels of FXIIa in response to an indication that a trigger has been input into the QSP model;
assigning a Factor XII trigger rate for one or more patients in a virtual patient population, wherein the Factor XII trigger rate comprises a rate at which autoactivation of Factor XII is triggered in the QSP model;
applying the QSP model to the one or more patients in the virtual patient population to obtain processed data, wherein the processed data comprises an amount of one or more contact system proteins;
determining an HAE attack frequency for the one or more patients in the virtual patient population based on the processed data; and
determining a relationship between HAE attack frequency and Factor XII trigger rate.

39. A computer-implemented method for determining an effectiveness of an administered drug in treating hereditary angioedema (HAE), the method comprising:
determining pharmacokinetic parameters of the administered drug for a virtual patient population;
determining disease predictive descriptors for the virtual patient population;
assigning the pharmacokinetic parameters and disease predictive descriptors to the virtual patient population;
processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and
using the processed data to obtain an indicator of the effectiveness of the administered drug on treating HAE.

40. The computer-implemented method of claim 39, wherein the processed data includes an amount of bradykinin.

41. The computer-implemented method of claim 39 or any other preceding claim, wherein the processed data includes an amount of cHMWK.

42. The computer-implemented method of claim 39 or any other preceding claim, wherein the processed data includes an amount of plasma kallikrein.

43. The computer-implemented method of claim 39 or any other preceding claim, wherein the indicator of the effectiveness of the administered drug is obtained at least in part by comparing the processed data to known data.

44. The computer-implemented method of claim 43 or any other preceding claim, wherein the known data comprises contact system protein amounts of an untreated subject with HAE.

45. The computer-implemented method of claim 43 or any other preceding claim, wherein the known data comprises contact system protein amounts of a subject without HAE.

46. The computer-implemented method of claim 39 or any other preceding claim, wherein the administered drug comprises Lanadelumab.

47. The computer-implemented method of claim 39 or any other preceding claim, wherein the administered drug comprises a small molecule PKa inhibitor.

48. The compute-implemented method of claim 39 or any other preceding claim, further comprising comparing the effectiveness of the administered drug to an effectiveness of a second drug.

49. The computer-implemented method of claim 39 or any other preceding claim, wherein the QSP model comprises a plurality of differential equations representing one or more biological reactions of a contact system.

50. The computer-implemented method of claim 39 or any other preceding claim, wherein the pharmacokinetic parameters comprise one or more parameters indicating how the administered drug is impacted by one or more biographical characteristics of a patient to whom the administered drug is administered.

51. The computer-implemented method of claim 50 or any other preceding claim, wherein the one or more biographical characteristics comprise at least one of height, weight, age, or gender.

52. The computer-implemented method of claim 39 or any other preceding claim, wherein the disease predictive descriptors comprise one or more parameters characterizing a propensity of a patient to experience an HAE flare-up.

53. The computer-implemented method of claim 52 or any other preceding claim, wherein the disease predictive descriptors include HAE flare-up frequency and/or severity.

54. The computer-implemented method of claim 39 or any other preceding claim, wherein the virtual patient population comprises a plurality of data sets, each data set of the plurality of data sets representing a virtual patient and having one or more variables defining one or more characteristics of the virtual patient.

55. The computer-implemented method of claim 54 or any other preceding claim, wherein the pharmacokinetic parameters and disease predictive parameters are assigned to the one or more variables of each data set.

56. A system, comprising
at least one computer hardware processor; and
at least one non-transitory computer-readable storage medium storing processor-executable instructions that, when executed by at least one computer hardware processor, cause the at least one computer-hardware processor to perform a method for determining an effectiveness of an administered drug in treating hereditary angioedema (HAE), the method comprising:
determining pharmacokinetic parameters of the administered drug for a virtual patient population;
determining disease predictive descriptors for the virtual patient population;
assigning the pharmacokinetic parameters and disease predictive descriptors to the virtual patient population;
processing the virtual patient population using a quantitative systems pharmacology (QSP) of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and
using the processed data to obtain an indicator of the effectiveness of the administered drug on treating HAE.

57. At least one non-transitory computer-readable storage medium storing processor-executable instructions that, when executed by at least one computer hardware processor, cause the at least one computer-hardware processor to perform a method for determining an effectiveness of an administered drug in treating hereditary angioedema (HAE), the method comprising:
determining pharmacokinetic parameters of the administered drug for a virtual patient population;
determining disease predictive descriptors for the virtual patient population;
assigning the pharmacokinetic parameters and disease predictive descriptors to the virtual patient population;
processing the virtual patient population using a quantitative systems pharmacology (QSP) model of HAE, wherein the QSP model is configured to represent autoactivation of Factor XII by elevating levels of FXIIa in response to an indication that a trigger has been input into the QSP model and the processed data comprises an amount of one or more contact system proteins; and
using the processed data to obtain an indicator of the effectiveness of the administered drug on treating HAE.

58. A computer-implemented method for determining an effectiveness of a dosage of an administered drug in treating hereditary angioedema (HAE), the method comprising:
determining pharmacokinetic parameters of the dosage of the administered drug for a virtual patient population;
determining disease predictive descriptors for the virtual patient population;
assigning the pharmacokinetic parameters and disease predictive descriptors to the virtual patient population;
processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and
using the processed data to obtain an indicator of the effectiveness of the dosage of the administered drug on treating HAE.

59. The computer-implemented method of claim 58, wherein the processed data includes an amount of bradykinin.

60. The computer-implemented method of claim 58 or any other preceding claim, wherein the processed data includes an amount of cHMWK.

61. The computer-implemented method of claim 58 or any other preceding claim, wherein the processed data includes an amount of plasma kallikrein.

62. The computer-implemented method of claim 58 or any other preceding claim, wherein the indicator of effectiveness of the dosage of the administered drug is obtained at least in part by comparing the processed data to known data.

63. The computer-implemented method of claim 62 or any other preceding claim, wherein the known data comprises contact system protein amounts of an untreated subject with HAE.

64. The computer-implemented method of claim 62 or any other preceding claim, wherein the known data comprises contact system protein amounts of a subject without HAE.

65. The computer-implemented method of claim 62 or any other preceding claim, wherein the known data comprises contact system protein amounts of a subject treated with a different dosage of the administered drug.

66. The computer-implemented method of claim 58 or any other preceding claim, wherein the administered drug comprises Lanadelumab.

67. The computer-implemented method of claim 58 or any other preceding claim, wherein the administered drug comprises a small molecule PKa inhibitor.

68. The computer implemented method of claim 58 or any other preceding claim, further comprising comparing the effectiveness of the dosage of the administered drug to an effectiveness of a different dosage of the administered drug.

69. The computer-implemented method of claim 58 or any other preceding claim, wherein the dosage comprises 150 milligrams every four weeks.

70. The computer implemented method of claim 58 or any other preceding claim, wherein the dosage comprises 300 milligrams every four weeks.

71. The computer-implemented method of claim 58 or any other preceding claim, wherein the dosage comprises 300 milligrams every two weeks.

72. The computer-implemented method of claim 58 or any other preceding claim, wherein the QSP model comprises a plurality of differential equations representing one or more biological reactions of a contact system.

73. The computer-implemented method of claim 58 or any other preceding claim, wherein the pharmacokinetic parameters comprise one or more parameters indicating how the administered drug is impacted by one or more biographical characteristics of a patient to whom the administered drug is administered.

74. The computer-implemented method of claim 73 or any other preceding claim, wherein the one or more biographical characteristics comprise at least one of height, weight, age, or gender.

75. The computer-implemented method of claim 58 or any other preceding claim, wherein the disease predictive descriptors comprise one or more parameters characterizing a propensity of a patient to experience an HAE flare-up.

76. The computer-implemented method of claim 75 or any other preceding claim, wherein the disease predictive descriptors include HAE flare-up frequency and/or severity.

77. The computer-implemented method of claim 58 or any other preceding claim, wherein the virtual patient population comprises a plurality of data sets, each data set of the plurality of data sets representing a virtual patient and having one or more variables defining one or more characteristics of the virtual patient.

78. The computer-implemented method of claim 77 or any other preceding claim, wherein the pharmacokinetic parameters and disease predictive parameters are assigned to the one or more variables of each data set.

79. A system, comprising:
at least one computer-hardware processor; and
at least one non-transitory computer-readable storage medium storing processor-executable instructions that, when executed by the at least one computer-hardware processor, cause the at least one computer-hardware processor to perform a method for determining an effectiveness of a dosage of an administered drug in treating hereditary angioedema (HAE), the method comprising:
determining pharmacokinetic parameters of the dosage of the administered drug for a virtual patient population;
determining disease predictive descriptors for the virtual patient population;
assigning the pharmacokinetic parameters and disease predictive descriptors to the virtual patient population;
processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and
using the processed data to obtain an indicator of the effectiveness of the dosage of the administered drug on treating HAE.

80. At least one non-transitory computer-readable storage medium storing processor-executable instructions that, when executed by at least one computer-hardware processor, cause the at least one computer-hardware processor to perform a method for determining an effectiveness of a dosage of an administered drug in treating hereditary angioedema (HAE), the method comprising:
determining pharmacokinetic parameters of the dosage of the administered drug for a virtual patient population;
determining disease predictive descriptors for the virtual patient population;
assigning the pharmacokinetic parameters and disease predictive descriptors to the virtual patient population;
processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and
using the processed data to obtain an indicator of the effectiveness of the dosage of the administered drug on treating HAE.

81. A computer-implemented method for determining an effect of non-adherence to a dosing regimen of an administered drug in treating hereditary angioedema (HAE), the method comprising:
determining pharmacokinetic parameters of the administered drug for a virtual patient population, wherein the pharmacokinetic parameters include a frequency of non-adherence to the dosing regimen;
determining disease predictive descriptors for the virtual patient population;
assigning the pharmacokinetic parameters and disease predictive descriptors to the virtual patient population;
processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and
using the processed data to determine an effect of the frequency of non-adherence on treating HAE.

82. The computer-implemented method of claim 81, wherein the processed data includes a HAE flare-up frequency.

83. The computer implemented method of claim 81 or any other preceding claim, wherein the processed data includes a HAE flare-up severity.

84. The computer-implemented method of claim 81 or any other preceding claim, wherein using the processed data to determine the effect of the frequency of non-adherence on treating HAE includes comparing the processed data to known data.

85. The computer-implemented method of claim 81 or any other preceding claim, wherein the QSP model comprises a plurality of differential equations representing one or more biological reactions of a contact system.

86. The computer-implemented method of claim 81 or any other preceding claim, wherein the pharmacokinetic parameters comprise one or more parameters indicating how the administered drug is impacted by one or more biographical characteristics of a patient to whom the administered drug is administered.

87. The computer-implemented method of claim 86 or any other preceding claim, wherein the one or more biographical characteristics comprise at least one of height, weight, age, or gender.

88. The computer-implemented method of claim 81 or any other preceding claim, wherein the disease predictive descriptors comprise one or more parameters characterizing a propensity of a patient to experience an HAE flare-up.

89. The computer-implemented method of claim 88 or any other preceding claim, wherein the disease predictive descriptors include HAE flare-up frequency and/or severity.

90. The computer-implemented method of claim 81 or any other preceding claim, wherein the virtual patient population comprises a plurality of data sets, each data set of the plurality of data sets representing a virtual patient and having one or more variables defining one or more characteristics of the virtual patient.

91. The computer-implemented method of claim 90 or any other preceding claim, wherein the pharmacokinetic parameters and disease predictive parameters are assigned to the one or more variables of each data set.

92. A system, comprising:
at least one computer hardware processor; and
at least one non-transitory computer-readable medium storing processor-executable instructions that, when executed by the at least one computer-hardware processor, cause the at least one computer-hardware processor to perform a method for determining an effect of non-adherence to a dosing regimen of an administered drug in treating hereditary angioedema (HAE), the method comprising:
determining pharmacokinetic parameters of the administered drug for a virtual patient population, wherein the pharmacokinetic parameters include a frequency of non-adherence to the dosing regimen;
determining disease predictive descriptors for the virtual patient population;
assigning the pharmacokinetic parameters and disease predictive descriptors to the virtual patient population;
processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and
using the processed data to determine an effect of the frequency of non-adherence on treating HAE.

93. At least one non-transitory computer-readable storage medium storing processor-executable instructions that, when executed by at least one computer-hardware processor, cause the at least one computer-hardware processor to perform a method for determining an effect of non-adherence to a dosing regimen of an administered drug in treating hereditary angioedema (HAE), the method comprising:
determining pharmacokinetic parameters of the administered drug for a virtual patient population, wherein the pharmacokinetic parameters include a frequency of non-adherence to the dosing regimen;
determining disease predictive descriptors for the virtual patient population;
assigning the pharmacokinetic parameters and disease predictive descriptors to the virtual patient population;
processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and
using the processed data to determine an effect of the frequency of non-adherence on treating HAE.

94. A computer-implemented method for determining an amount of a protein of a contact system in a patient in response to administration of a drug for treating hereditary angioedema (HAE), the method comprising:
determining pharmacokinetic parameters of the drug for a virtual patient population;
determining disease predictive descriptors for the virtual patient population;
assigning the pharmacokinetic parameters and disease predictive descriptors to the virtual patient population;
processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model; and
determining the amount of the protein based on the processed data.

95. The computer-implemented method of claim 94, wherein the protein comprises bradykinin.

96. The computer-implemented method of claim 94 or any other preceding claim, wherein the protein comprises cHMWK.

97. The computer-implemented method of claim 94 or any other preceding claim, wherein the protein comprises plasma kallikrein.

98. The computer-implemented method of claim 94 or any other preceding claim, wherein the drug comprises Lanadelumab.

99. The computer-implemented method of claim 94 or any other preceding claim, wherein the drug comprises a small molecule PKa inhibitor.

100. The computer-implemented method of claim 94 or any other preceding claim, further comprising, using the amount of the protein to determine an effectiveness of the drug.

101. The computer-implemented method of claim 94 or any other preceding claim, further comprising using the amount of the protein to determine whether an HAE flare-up has occurred.

102. The computer-implemented method of claim 101 or any other preceding claim, wherein using the amount of the protein to determine whether an HAE flare-up has occurred comprises comparing the amount of the protein to a known threshold.

103. The computer-implemented method of claim 94 or any other preceding claim, further comprising, using the amount of the protein to determine a HAE flare-up frequency.

104. The computer-implemented method of claim 94 or any other preceding claim, wherein the QSP model comprises a plurality of differential equations representing one or more biological reactions of the contact system.

105. The computer-implemented method of claim 94 or any other preceding claim, wherein the pharmacokinetic parameters comprise one or more parameters indicating how the administered drug is impacted by one or more biographical characteristics of the patient to whom the administered drug is administered.

106. The computer-implemented method of claim 105 or any other preceding claim, wherein the one or more biographical characteristics comprise at least one of height, weight, age, or gender.

107. The computer-implemented method of claim 94 or any other preceding claim, wherein the disease predictive descriptors comprise one or more parameters characterizing a propensity of the patient to experience an HAE flare-up.

108. The computer-implemented method of claim 107 or any other preceding claim, wherein the disease predictive descriptors include HAE flare-up frequency and/or severity.

109. The computer-implemented method of claim 94 or any other preceding claim, wherein the virtual patient population comprises a plurality of data sets, each data set of the plurality of data sets representing a virtual patient and having one or more variables defining one or more characteristics of the virtual patient.

110. The computer-implemented method of claim 109 or any other preceding claim, wherein the pharmacokinetic parameters and disease predictive parameters are assigned to the one or more variables of each data set.

111. A system, comprising:
at least one computer-hardware processor; and
at least one non-transitory computer-readable storage medium storing processor-executable instructions that, when executed by the at least one computer-hardware processor, cause the at least one computer-hardware processor to perform a method for determining an amount of a protein of a contact system in a patient in response to administration of a drug for treating hereditary angioedema (HAE), the method comprising:
determining pharmacokinetic parameters of the drug for a virtual patient population;
determining disease predictive descriptors for the virtual patient population;
assigning the pharmacokinetic parameters and disease predictive descriptors to the virtual patient population;
processing the virtual patient population using a quantitative systems pharmacology (QSP) model of HAE obtain processed data, wherein the QSP model is configured to represent autoactivation of Factor XII by elevating levels of FXIIa in response to an indication that a trigger has been input into the QSP model; and
determining the amount of the protein based on the processed data.

112. At least one non-transitory computer-readable storage medium storing processor-executable instructions that, when executed by at least one computer-hardware processor, cause the at least one computer-hardware processor to perform a method for determining an amount of a protein of a contact system in a patient in response to administration of a drug for treating hereditary angioedema (HAE), the method comprising:
determining pharmacokinetic parameters of the drug for a virtual patient population;
determining disease predictive descriptors for the virtual patient population;
assigning the pharmacokinetic parameters and disease predictive descriptors to the virtual patient population;
processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model; and
determining the amount of the protein based on the processed data.

113. A computer-implemented method for determining a temporal profile illustrating an effect of a drug on a contact system in a patient, the method comprising:
determining pharmacokinetic parameters of the drug for a virtual patient population;
determining disease predictive descriptors for the virtual patient population;
assigning the pharmacokinetic parameters and disease predictive descriptors to the virtual patient population;
processing the virtual patient population using a quantitative systems pharmacology (QSP) model of hereditary angioedema (HAE) to obtain processed data, wherein the QSP model is configured to represent autoactivation of Factor XII by elevating levels of FXIIa in response to an indication that a trigger has been input into the QSP model and the processed data comprises an amount of one or more contact system proteins; and
using the processed data to determine a measure of an amount of one or more proteins of the contact system over time in response to the drug.

114. The computer-implemented method of claim 113, wherein the one or more proteins comprise at least one member selected from the group comprising bradykinin, plasma kallikrein, and cHMWK.

115. The computer-implemented method of claim 113 or any other preceding claim, further comprising using the processed data to obtain a measure of HAE flare-up severity over time in response to the drug.

116. The computer-implemented method of claim 113 or any other preceding claim, further comprising using the processed data to obtain a measure of HAE flare-up frequency over time in response to the drug.

117. The computer-implemented method of claim 113 or any other preceding claim, wherein the QSP model comprises a plurality of differential equations representing one or more biological reactions of the contact system.

118. The computer-implemented method of claim 113 or any other preceding claim, wherein the pharmacokinetic parameters comprise one or more parameters indicating how the administered drug is impacted by one or more biographical characteristics of a patient to whom the administered drug is administered.

119. The computer-implemented method of claim 118 or any other preceding claim, wherein the one or more biographical characteristics comprise at least one of height, weight, age, or gender.

120. The computer-implemented method of claim 113 or any other preceding claim, wherein the disease predictive descriptors comprise one or more parameters characterizing a propensity of a patient to experience an HAE flare-up.

121. The computer-implemented method of claim 120 or any other preceding claim, wherein the disease predictive descriptors include HAE flare-up frequency and/or severity.

122. The computer-implemented method of claim 113 or any other preceding claim, wherein the virtual patient population comprises a plurality of data sets, each data set of the plurality of data sets representing a virtual patient and having one or more variables defining one or more characteristics of the virtual patient.

123. The computer-implemented method of claim 122 or any other preceding claim, wherein the pharmacokinetic parameters and disease predictive parameters are assigned to the one or more variables of each data set.

124. A system, comprising:
at least one computer-hardware processor; and
at least one non-transitory computer-readable storage medium storing processor-executable instruction that, when executed by the at least one computer-hardware processor, cause the at least one computer-hardware processor to perform a method for determining a temporal profile illustrating an effect of a drug on a contact system in a patient, the method comprising:
determining pharmacokinetic parameters of the drug for a virtual patient population;
determining disease predictive descriptors for the virtual patient population;
assigning the pharmacokinetic parameters and disease predictive descriptors to the virtual patient population;
processing the virtual patient population using a quantitative systems pharmacology (QSP) model of hereditary angioedema (HAE) to obtain processed data, wherein the QSP model is configured to represent autoactivation of Factor XII by elevating levels of FXIIa in response to an indication that a trigger has been input into the QSP model and the processed data comprises an amount of one or more contact system proteins; and
using the processed data to determine a measure of an amount of one or more proteins of the contact system over time in response to the drug.

125. At least one non-transitory computer-readable storage medium storing processor-executable instruction that, when executed by at least one computer-hardware processor, cause the at least one computer-hardware processor to perform a method for determining a temporal profile illustrating an effect of a drug on a contact system in a patient, the method comprising:
determining pharmacokinetic parameters of the drug for a virtual patient population;
determining disease predictive descriptors for the virtual patient population;
assigning the pharmacokinetic parameters and disease predictive descriptors to the virtual patient population;
processing the virtual patient population using a quantitative systems pharmacology (QSP) model of hereditary angioedema (HAE) to obtain processed data, wherein the QSP model is configured to represent autoactivation of Factor XII by elevating levels of FXIIa in response to an indication that a trigger has been input into the QSP model and the processed data comprises an amount of one or more contact system proteins; and
using the processed data to determine a measure of an amount of one or more proteins of the contact system over time in response to the drug.

126. A computer-implemented method for determining a characteristic of a hereditary angioedema (HAE) flare-up in response to administering a drug to a patient, the method comprising:
determining pharmacokinetic parameters of the drug for a virtual patient population;
determining disease predictive descriptors for the virtual patient population;
assigning the pharmacokinetic parameters and disease predictive descriptors to the virtual patient population;
processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and
using the processed data to determine the characteristic of the HAE flare-up in response to administering the drug to the patient.

127. The computer-implemented method of claim 126, wherein the characteristic of the HAE flare-up comprises HAE flare-up severity.

128. The computer-implemented method of claim 126 or any other preceding claim, wherein the characteristic of the HAE flare-up comprises HAE flare-up frequency.

129. The computer-implemented method of claim 126 or any other preceding claim, wherein the characteristic of the HAE flare-up comprises HAE flare-up duration.

130. The computer-implemented method of claim 126 or any other preceding claim, wherein:
the pharmacokinetic parameters include a dosage of the drug; and
the method further comprises using the processed data to determine the characteristic of the HAE flare-up in response to administering the dosage of the drug to the patient.

131. The computer-implemented method of claim 126 or any other preceding claim, wherein the drug comprises Lanadelumab.

132. The computer-implemented method of claim 126 or any other preceding claim, wherein the drug comprises a small molecule PKa inhibitor.

133. The computer-implemented method of claim 126 or any other preceding claim, wherein the QSP model comprises a plurality of differential equations representing one or more biological reactions of a contact system.

134. The computer-implemented method of claim 126 or any other preceding claim, wherein the pharmacokinetic parameters comprise one or more parameters indicating how the administered drug is impacted by one or more biographical characteristics of the patient to whom the administered drug is administered.

135. The computer-implemented method of claim 134 or any other preceding claim, wherein the one or more biographical characteristics comprise at least one of height, weight, age, or gender.

136. The computer-implemented method of claim 126 or any other preceding claim, wherein the disease predictive descriptors comprise one or more parameters characterizing a propensity of the patient to experience an HAE flare-up.

137. The computer-implemented method of claim 136 or any other preceding claim, wherein the disease predictive descriptors include HAE flare-up frequency and/or severity.

138. The computer-implemented method of claim 126 or any other preceding claim, wherein the virtual patient population comprises a plurality of data sets, each data set of the plurality of data sets representing a virtual patient and having one or more variables defining one or more characteristics of the virtual patient.

139. The computer-implemented method of claim 138 or any other preceding claim, wherein the pharmacokinetic parameters and disease predictive parameters are assigned to the one or more variables of each data set.

140. A system, comprising:
at least one computer-hardware processor;
at least one non-transitory computer-readable hardware medium storing processor-executable instructions that, when executed by the at least one computer-hardware processor, cause the at least one computer-hardware processor to perform a method for determining a characteristic of a hereditary angioedema (HAE) flare-up in response to administering a drug to a patient, the method comprising:
determining pharmacokinetic parameters of the drug for a virtual patient population;
determining disease predictive descriptors for the virtual patient population;
assigning the pharmacokinetic parameters and disease predictive descriptors to the virtual patient population;
processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and
using the processed data to determine the characteristic of the HAE flare-up in response to administering the drug to the patient.

141. At least one non-transitory computer-readable hardware medium storing processor-executable instructions that, when executed by at least one computer-hardware processor, cause the at least one computer-hardware processor to perform a method for determining a characteristic of a hereditary angioedema (HAE) flare-up in response to administering a drug to a patient, the method comprising:
determining pharmacokinetic parameters of the drug for a virtual patient population;
determining disease predictive descriptors for the virtual patient population;
assigning the pharmacokinetic parameters and disease predictive descriptors to the virtual patient population;
processing the virtual patient population using a quantitative systems pharmacology (QSP) model of HAE to obtain processed data, wherein the QSP model is configured to represent autoactivation of Factor XII by elevating levels of FXIIa in response to an indication that a trigger has been input into the QSP model and the processed data comprises an amount of one or more contact system proteins; and
using the processed data to determine the characteristic of the HAE flare-up in response to administering the drug to the patient.

142. A method for developing a virtual patient population comprising a plurality of virtual patients for input into a quantitative systems pharmacology (QSP) model of hereditary angioedema (HAE), wherein the QSP model is configured to represent autoactivation of Factor XII by elevating levels of FXIIa in response to an indication that a trigger has been input into the QSP model and to output an amount of one or more contact system proteins, the method comprising:
assigning pharmacokinetic parameters to the virtual patient population;
determining a baseline attack frequency and baseline attack severity for each patient in the virtual patient population; and
assigning the baseline attack frequency and baseline attack severity to each patient in the virtual patient population.

143. The method of claim 142, further comprising inputting the virtual patient population into the quantitative systems pharmacology (QSP) model for HAE.

144. The method of claim 142, wherein the baseline attack frequency is determined at least in part by using a Poisson process informed by known data.

145. The method of claim 142 or any other preceding claim, wherein the baseline attack frequency comprises an attack frequency in an untreated patient and the baseline attack severity comprises an attack severity in the untreated patient.

146. The computer-implemented method of claim 142 or any other preceding claim, wherein the virtual patient population comprises a plurality of data sets, each data set of the plurality of data sets representing a virtual patient of the plurality of virtual patients of the virtual patient population and having one or more variables defining one or more characteristics of the virtual patient.

147. The computer-implemented method of claim 146 or any other preceding claim, wherein the pharmacokinetic parameters, baseline attack frequency, and baseline attack severity are assigned to the one or more variables of each data set.

148. A system, comprising:
at least one computer-hardware processor;
at least one non-transitory computer-readable storage medium storing processor-executable instructions that, when executed by the at least one computer-hardware processor, cause the at least one computer-hardware processor to perform a method for developing a virtual population for input into a quantitative systems pharmacology (QSP) model of hereditary angioedema (HAE), wherein the QSP model is configured to represent autoactivation of Factor XII by elevating levels of FXIIa in response to an indication that a trigger has been input into the QSP model to output an amount of one or more contact system proteins, the method comprising:
assigning pharmacokinetic parameters to the virtual patient population;
determining a baseline attack frequency and a baseline attack severity for each patient in the virtual patient population; and
assigning the baseline attack frequency and baseline attack severity to each patient in the virtual patient population.

149. At least one non-transitory computer-readable storage medium storing processor-executable instructions that, when executed by at least one computer-hardware processor, cause the at least one computer-hardware processor to perform a method for developing a virtual population for input into a quantitative systems pharmacology (QSP) model of hereditary angioedema (HAE), wherein the QSP model is configured to represent autoactivation of Factor XII by elevating levels of FXIIa in response to an indication that a trigger has been input into the QSP model to output an amount of one or more contact system proteins, the method comprising:
assigning pharmacokinetic parameters to the virtual patient population;
determining a baseline attack frequency and a baseline attack severity for each patient in the virtual patient population; and
assigning the baseline attack frequency and baseline attack severity to each patient in the virtual patient population.
